(19) United States
(12) Patent Application Publication
(10) Pub. No.: US 2026/0337239 A1
(43) Pub. Date: Oct. 29, 2026

(54) CHEMICALLY STABLE FLUORESCENT PROTEINS FOR ADVANCED MICROSCOPY

(71) Applicant: Cornell University, Ithaca, NY (US)

(72) Inventors: Benjamin C. Campbell, San Diego, CA (US); Gregory A. Petsko, Manchester-By-The-Sea, MA (US)

(21) Appl. No.: 18/993,898

(22) PCT Filed: Jul. 11, 2023
(86) PCT No.: PCT/US2023/027373

§ 371 Date: Jan. 13, 2025

Related U.S. Application Data
(60) Provisional application No. 63/388,051, filed on Jul. 11, 2022.

(51) Int. Cl. C07K 14/435; C07K 1/14; G01N 21/64

(52) U.S. Cl. CPC C07K 14/43595; C07K 1/14; G01N 21/6428; C07K 2319/60; G01N 2021/6439

(57) ABSTRACT

This disclosure provides fluorescent proteins that withstand chaotropic conditions that denature most biological structures within seconds, including superfolder GFP. The disclosed fluorescent proteins contain no cysteines, is chloride insensitive, and tolerates aldehyde and osmium tetroxide fixation better than common fluorescent proteins. The disclosed fluorescent proteins represent a new generation of robustly stable fluorescent proteins developed for advanced biotechnological applications

### RELATED APPLICATIONS

This patent application claims the benefit of U.S. Provisional Patent Application No. 63/388,051, filed Jul. 11, 2023, which is hereby incorporated by reference in its entirety.

### BACKGROUND

Fluorescent proteins (FPs) have been used for nearly three decades to probe cell biology, but the technology is still catching up with striking advances in microscopy methods like super-resolution (SR) imaging, expansion microscopy (ExM), and correlative light and electron microscopy (CLEM). FPs can be adversely affected (sometimes dramatically) by even simple chemical fixation, and many do not function well in the hydrogel-enmeshed samples of ExM. Correlative light and electron microscopy (CLEM) places even greater demands on FPs, as it usually involves secondary fixation and staining with caustic chemicals such as osmium tetroxide (OsO₄). In view of the foregoing, there is a need for new FPs that could be used in protein-retention expansion microscopy (proExM) and correlative light and electron microscopy (CLEM).

### BRIEF SUMMARY

The present disclosure is generally directed to a fluorescent protein comprising at least 90%, 95%, 96%, 97%, 98%, 99% or 100% of the amino acid sequence selected from SEQ ID NOs: 1, 3, 5, 7-10, and 12. In some embodiments, the fluorescent protein comprises the amino acid sequence of any one of SEQ ID NOs: 1, 3, 5, 7-10, and 12. In some embodiments, the fluorescent protein is chloride insensitive.

The disclosure also encompasses a fusion protein comprising the fluorescent protein disclosed herein. In some embodiments, the fluorescent protein is linked to a polypeptide.

The disclosure also encompasses a nucleic acid molecule encoding the fluorescent protein disclosed herein.

The disclosure also encompasses a nucleic acid molecule encoding the fusion protein disclosed herein.

The disclosure also encompasses a nucleic acid molecule comprising the nucleotide sequence selected from SEQ ID NOs: 2, 4, and 6. In some embodiments, the nucleic acid molecule is a cDNA.

The disclosure also encompasses a vector comprising the nucleic acid molecule disclosed herein.

The disclosure also encompasses a host cell comprising the nucleic acid molecule disclosed herein.

The disclosure also encompasses a host cell comprising the vector disclosed herein.

The disclosure also encompasses a host cell comprising the fluorescent protein disclosed herein.

The disclosure also encompasses a host cell comprising the fusion protein disclosed herein. In some embodiments, the host ccli is a mammalian host cell.

The disclosure also encompasses a method of detecting the expression of a protein of interest in a cell. The method comprises introducing into a cell a nucleic acid molecule comprising a nucleotide sequence encoding a protein of interest fused to a nucleotide sequence encoding the fluorescent protein disclosed herein. The method may further comprise culturing the cell under conditions suitable for the expression of said protein of interest. The method may further comprise detecting the expression of said protein of interest by measuring the fluorescence of said cell. In some embodiments, said nucleic acid molecule being operatively linked and under the control of a suitable expression control sequence. In some embodiments, the fluorescence is measured by optical means. In some embodiments, the optical means is fluorescent microscopy, flow cytometry, spectroscopy, laser-scanning confocal microscope, confocal microscope, correlative light and electron microscopy, or protein-retention expansion microscopy.

The disclosure also encompasses a method of purifying a protein of interest. The method comprises introducing into a cell a nucleic acid molecule comprising a nucleotide sequence encoding a protein of interest fused to a nucleotide sequence encoding the fluorescent protein disclosed herein. The method may further comprise culturing the cell under conditions suitable for the expression of said protein of interest. The method may further comprise lysing the cell. The method may further comprise purifying the protein of interest by purifying the fluorescent protein. In some embodiments, said nucleic acid molecule being operatively linked and under the control of a suitable expression control sequence. In some embodiments, the fluorescent protein is purified by organic extraction or Fluorescence ImmunoPrecipitation (FLIP).

### BRIEF DESCRIPTION OF THE DRAWINGS/FIGURES

FIGS. 1A-1M shows biochemical characterization of hyperfolder YFP and its performance in cells. FIG. 1A shows excitation and emission spectrum of hfYFP. FIG. 1B shows absorbance spectrum at pH 7.5. Arrow indicates the non-excitable 390-405 nm band present in eYFP and absent in hfYFP and mhYFP. The latter two spectra overlap. FIG. 1C shows chromophore maturation in bacterial lysate. n=3 experiments, mean±s.d. FIG. 1D shows fluorescence of FP-expressing bacteria after overnight growth at 37° C. FIG. 1E shows fluorescence of HeLa, BE(2)-M17, and HEK293T cells 48 hr after chemical transfection using a plasmid containing a P2A peptide to generate each FP and an mCherry reference in a roughly equimolar ratio (inset:plasmid). Data for each FP is normalized to the mCherry signal and plotted relative to eGFP. n=4 experiments, each an average of 3 replicate transfections, mean±s.e.m. FIG. 1F shows pH titration of hfYFP and eYFP. n=1 experiment with three technical replicates averaged FIG. 1G shows chloride titration in solutions of HEPES-NaOH, pH 7.5. Data points are fit to a simple linear regression. n=3, mean±s.d. FIGS. 1H-1M show live HeLa cells imaged after overnight transfection using plasmids encoding: FIG. 1H shows LifeAct-7aahfYFP; actin. FIG. 1I shows hfYFP-6aa-tubulin; tubulin. FIG. 1J shows hfYFP-15aa-clathrin; clathrin. FIG. 1K shows pCytERM-hfYFP; endoplasmic reticulum. FIG. 1I shows COX8A[x4]-4aa-hfYFP; mitochondria, here shown in BE(2)-M17 cells. FIG. 1M shows 12B-6aa-hfYFP; nucleus, in HeLa cells. Scale bars: 10 um

FIGS. 2A-2H show stability of purified fluorescent proteins in chaotropic conditions. FIG. 2A shows kinetic unfolding in 6.3 M buffered guanidinium HCI (GdnHCI) solution, pH 7.5. Inset figure: first 10 min of the same data set. Inset photograph: fluorescence of 1 uM purified superfolder GFP (sfGFP) and hyperfolder YFP (hfYFP) protein after 3 months in 6.3 M GdnHCI solution at room temperature (RT), protected from light. FIG. 2B shows kinetic unfolding in 3.6 M buffered guanidinium thiocyanate (GdnSCN), pH 7.5. Inset: first 60 s. Every individual data point in FIGS. 2A-2B is normalized to native protein run in parallel under identical conditions in the same buffer without Gdn. FIG. 2C shows equilibrium unfolding in GdnHCI and FIG. 2D shows in GdnSCN, at 24 hr. Fluorescence is normalized to the intensity value for each FP at 0 M Gdn; mean±s.d. FIG. 2E shows fluorescence intensity during isothermal melting at 87° C., relative to time zero, with normalization as described in FIGS. 2A-2B shows. Inset: first 8 min of the data set. FIG. 2F shows isothermal melting after 60 min exposure to various temperatures. Data are plotted relative to the intensity value for each FP at 25° C. See FIG. 13 for the complete data set. FIG. 2G shows fluorescence during a 0.3° C./min temperature ramp from 25-100° C., with temperature range 60-100° C. displayed. Data are normalized to the intensity values at 25° C. for the individual FPs. FIG. 2H shows intensity versus H₂O₂ concentration in buffered solution after exactly 15 min incubation at RT. Double-exponential curve fits are shown with data points; n=3 replicate experiments, mean±s.d. The same 7 FPs are plotted in every panel in this figure, including those that denatured immediately (y=0).

FIGS. 3A-3E show fluorescence retained by transfected human cells after expansion microscopy and fixation. FIG. 3A shows Fluorescence retained by HEK293T cells after fixation using room temperature 4% PFA in PBS, pH 7.4, or b, 4% PFA+5% Glut in PBS, pH 7.4. Cytosolic expression, n>227 cells for each condition per FP. FIG. 3C shows representative images of HEK293T cells captured on a widefield microscope using the same acquisition settings before and after fixation as in FIG. 3B. Retained fluorescence (%) is indicated below the fixed images in gray. Scale bars, 20 pm. Cytosolic expression. FIG. 3DA shows HeLa cells transfected with LifeAct-mhYFP were imaged on a confocal microscope before proExM (left); after partial expansion of the hydrogelenmeshed sample using PBS (middle); and after full expansion using dd-H20 (right). All images were acquired at 63× magnification. FIG. 3E shows fluorescence retained by H2B-FP transfected HeLa cells in hypertonic “shrinking solution” after full expansion in proExM, relative to the same live cells (Methods). One-way ANOVA with multiple comparisons, ***p=0.0007, “p=0.0189. Complete statistics for FIGS. 3A, B, and E are available in FIG. 24. Abbreviations: proExM, protein-retention expansion microscopy; PFA, paraformaldehyde; Glut, glutaraldehyde; PBS, phosphate buffered saline; mNG, mNeonGreen; mClo3, mClover3; mGL, mGreenLantern; hfYFP, hyperfolder YFP

FIGS. 4A-4H show resilience of hfYFP during electron microscopy preservation. FIG. 4A shows Osmium tetroxide (OsO₄) dose-response curve using purified FPs after 1 hr incubation at RT. n=3 replicate experiments, mean±s.e.m. FIG. 4B shows fluorescence of purified FPs in 1% OsO₄, recorded at 10 min intervals. n=3 replicate experiments, mean±s.e.m. FIG. 4C shows workflow for evaluating the performance of FPs in electron microscopy (EM). FPs were expressed in the cytoplasm using adeno-associated virus (AAV) transduction and imaged using confocal microscopy. Cultures were then post-fixed with EM fixative (4% paraformaldehyde and 0.2% glutaraldehyde), harvested in bovine serum albumin (BSA), and embedded in agarose. The agarose-embedded cells were incubated in 1% OsO₄ solution for 1 hr, after which the osmicated tissue in agarose was embedded in OCT (optimal cutting temperature) compound on dry ice for cryosectioning. Images of mounted cryosections were collected using the same settings as for live imaging, to evaluate fluorescence retention. FIG. 4D shows HEK293T cells imaged using confocal microscopy and 488 nm laser excitation after AAV transduction of cytosolic eGFP, mGL, or hfYFP. Magnification, 10×. Imaging parameters are identical between FPs. FIG. 4E shows representative images of cultures viewed at 63× magnification with DAPI staining. Imaging parameters are identical between FPs and are different than those used in FIG. 4E. FIG. 4F shows background-subtracted mean fluorescence intensity units (MIU) of live and osmicated-and-OCT-embedded cultures, averaged from 10 ROIs per FP, per condition. These raw intensity values should not be used for brightness comparison because different settings were used for each FP. FIG. 4G shows cellular fluorescence retention after OsO₄ incubation and OCT embedding, expressed as a percentage relative to the same live cells. FIG. 4H left shows Toluidine Blue staining was used to verify the presence of cells before preparation. Cells were fixed in EM aldehyde fixative, followed by high-pressure freezing and freeze substitution, 1% OsO₄ incubation, dehydration with 100% acetone, HM20 resin infiltration, and UV polymerization. FIG. 4H right shows laser scanning confocal images show 100 nm thick sections of fluorescent HEK293 cells expressing eGFP (top), mGL (middle), and hfYFP (bottom). Scale bars, 20 pm, unless otherwise indicated. mGL: mGreenLantern. mNG: mNeonGreen. hfYFP: hyperfolder YFP. mhYFP: monomeric hyperfolder YFP. sfGFP: superfolder GFP.

FIGS. 5A-5H show structure-guided engineering of large Stokes shift GFPs. FIGS. 5A-5B show hfYFP crystal structure. Residues on 13-strands 10 (cyan) 11 (magenta) that were targeted to generate the LSS-FP libraries are indicated. Dashes: hydrogen bonds. c, Excitation spectra of mGL, LSSmGFP, and LSSA12. The latter two FP spectra overlap. FIG. 5D shows excitation spectra of mT-Sapphire and eGFP. Arrows indicate wavelength ranges where cross-excitation would be expected from typical 405 nm or 470-491 nm excitation sources. FIG. 5E shows live HeLa cells transfected with LifeAct-eGFP and H2B-mT-Sapphire or LifeAct-mGL and H2B-LSSmGFP. Excitation at 470 nm co-excites mT-Sapphire and eGFP (white arrow) whereas LSSmGFP is not excited by 470 nm. Scale bars: 25 um. FIG. 5F shows benchtop fluorescence-assisted purification of SAV from E. coli inclusion bodies using eGFP, hfYFP, or LSSmGFP fusions as depicted in FIG. 5G. eGFP is immediately denatured during inclusion body solubilization with 6 M GdnHCI (arrow) and never regains fluorescence. hfYFP and LSSmGFP remain fluorescent throughout all stages of denaturing Ni-NTA purification (6 M GdnHCI present in all solutions), dialysis-based refolding of SAV in native buffer, enzymatic cleavage with His6-TEV protease, and re-isolation of cleaved SAV in native buffer by Ni-NTA chromatography (see FIG. 22 for workflow). hfYFP is illuminated using a 470 nm LED and photographed through a long-pass filter; LSSmGFP: 405 nm LED excitation without emission filter. FIG. 5G shows the fusion construct used for purification contains an N-terminal hexahistidine (His6)-tagged hfYFP (or eGFP or LSSmGFP for the example in FIG. 5F) and Cterminal Protein of Interest (POI) separated by a flexible linker containing a TEV cleavage site. After cleavage, His6-TEV and His6-hfYFP are adsorbed to Ni-NTA resin and the flow-through is collected to obtain the POI. FIG. 5H shows fluorescence of biotin-4-fluorescein is quenched upon SAV binding (see Kada et al., Biochimica et Biophysica Acta, 1999). The refolded SAV protein isolated after cleavage was active.

FIGS. 6A-6B show protein quantified from expression of fusion constructs in E. coll. FIG. 6A shows coomassie gels of soluble protein, insoluble protein, and protein from the media of the same cultures (without cells). Equal quantity of protein was run in each lane as determined by BCA assay, except for the media condition, where equal volume was used without adjustment. The molecular weight (MW) predicted by ExPASy for the FP fusions with mScarlet-1 (mSca), Bacillus circulans xylanase (Bcx), and streptavidin (SAV) are approximately 57 kDa, 51 kDa, and 44 kDa, respectively. The MW of an avFP is −27 kDa. FIG. 6B shows quantification of the protein fusion bands from gels in a using ImageJ densitometry tools. Numbers above the bar graph: calculated ratio of soluble protein to insoluble protein from raw densitometry values. n=3, mean±s.d.

FIGS. 7A-7E show characterization of cysteine-free mutants. FIG. 7A shows crystal structure of Clover (PDB ID: 5WJ2), with cysteines indicated. In all avFPs, C48 and C70 are situated −24 A apart (dotted line) and cannot form a disulfide bond under native conditions in properly folded protein. FIG. 7B shows comparison of cellular brightness values between “wild-type” FPs and cysteine-free variants (Methods). n=4 experimental replicates, each averaging 3 transfections, mean±s.e.m. FIG. 7C shows fluorescence of FPs during an 0.3 “C/min temperature ramp from 25-100° C. using a real-time PCR machine with FAM filter. Data are normalized to the fluorescence intensity value at 25° C. The black arrow indicates a prolonged secondary melting phase in Clover-C48S/C7OV that was not observed in the other FPs. The Tm=x when y=1. FIG. 7D shows data from FIG. 7C plotted as the negative first derivative of the change in fluorescence, normalized to max for each IP (arbitrary units) and plotted. FIG. 7E shows spectroscopic characterization of cysteine-free mutants and their brightness in HEK293T cells. The same data for eGFP, sfGFP, and Clover are shown in Table 2. Asterisk (*): human codon optimized sfGFP-C48S/C7OS is “moxGFP,” from Costantini et al., Nat. Comm., 2015, and the quantum yield ($) value is cited from that study; however, we recommend treating that value as an estimate, since their methods for QY determination were not reported. All other data in the table were produced in our lab in this study.

FIG. 8 shows mutation map of spectroscopically characterized hyperfolder GFP/YFP mutants. Green and yellow arrows indicate the cumulative mutations and steps leading to mGreenLantern and the final hyperfolder YFP (hfYFP) variants, respectively. Italicized mutations signify reversion at that site to the “wild-type” Clover amino acid identity. *S147R and L195M are de novo mutations originating in this study from PCR errors that occurred during site-directed mutagenesis. The S147R mutation was independently reported by another research group for a different purpose during the preparation of this manuscript (Thong et al., J. Neurosci. Met., 2019).

FIGS. 9A-9D show screening of fluorescent protein libraries. FIG. 9A shows melting curves for a subset of C48S/C7OV and FIG. 9B shows yellow fluorescent mutants compared to eGFP and eYFP. FIG. 9C shows cellular brightness for each FP in three mammalian cell lines (Methods). n=3 replicate experiments, each averaging 4 independent transfections, mean±s.e.m. mF6-HL and mfoxYY were not tested in BE(2)-M17 cells. FIG. 9D shows kinetic unfolding of several purified FPs in 6.3 M buffered GdnHCI, pH 7.4. Each data point is normalized to that of the same FP in buffer without Gdn, which was run in parallel. FPs containing cysteine substitutions are indicated. One-phase nonlinear regression is plotted for all FPs except for those with slope=0 (i.e., denatured instantly). FPs that denatured instantly (y=0 for x 0) upon exposure to GdnHCI are indicated by a colored dot instead of a line. n=3, mean±s.d. “Clover-cc” signifies the C48S/C7OV mutations to distinguish it from unmodified Clover in this figure.

FIGS. 10A-10B show effect of cysteine residues on refolding. FIG. 10A shows refolding of FPs after denaturation, relative to untreated native samples. Purified FPs (1 uM concentration) were melted at 95° C. for 10 min in the presence of 6.3 M GdnHCI, pH 7.5, and allowed to cool to room temperature (RT) before refolding was initiated by 10-fold dilution into fresh buffer lacking GdnHCI for 0.1 uM final protein concentration. n=3, mean±s.d. FIG. 10B shows refolding performed as before, with addition of reducing agent dithiothreitol (DTT) in the GdnHCI and refolding buffers. Final concentrations: [GdnHCI]=0.63 M, [DTT]=1 mM. All data points are shown, with double exponential curve fits superimposed. n=3, mean±s.d.

FIGS. 11A-11B show protein yield from E. coli lysate. FIG. 11A shows coomassie gel of soluble protein extracted from E. coli lysate after overnight expression. Black arrowhead indicates the fluorescent protein band. The same quantity of protein was run in each lane, so the band intensity at −27 kDa indicates soluble FP yield. FIG. 11B shows quantification of the −27 kDa band using ImageJ's densitometry tools. The total protein extracted from each culture is the sum of the soluble and insoluble fractions (total bar graph height). n=3, mean±s.d.

FIGS. 12A-12C show determination of FP oligomeric state. FIG. 12A shows gel filtration chromatography using 10 uM purified fluorescent protein in a Superdex S200 Increase 10/300 GL sizing column with elution monitored at 280 nm. The monomer fraction elutes at −17.0 mL and the dimer −15.5 mL. Data are normalized to the maximum A280 value for each FP. FIG. 12B shows representative HeLa cells imaged 16 hr after chemical transfection with CytERM-FP plasmids. The organized smooth endoplasmic reticulum assay (OSER) is a cellular FP aggregation protocol. Cells with large, bright aggregates, like those in tdTomato, are scored as “OSER.” Normal cells have healthy reticular ER structure, nuclei, and no OSER structures (see Methods; refer to definitions by Costantini et al., Traffic, 2012). Scale bars, 25 pm. mGL: mGreenLantern. hfYFP: hyperfolder YFP. hfYFP-K: hfYFP-V206K. mhYFP: monomeric hyperfolder YFP. tdTomato: tandem dimer Tomato.

FIG. 13 shows isothermal melting of fluorescent proteins. FPs were rapidly heated to the target temperatures in individual wells using the gradient function of a real-time PCR machine. Fluorescence intensity was quantified every 30 s using the FAM filter. Data are plotted by normalizing the intensity values to the first data point at t=0 min. Values from the same data set at t=60 min were used to construct the summary panel in FIG. 2f. mGL: mGreenLantern. hfYFP: hyperfolder YFP.

FIGS. 14A-14K show Behavior of FPs in sodium hydroxide solution. FIG. 14A Clover and FIG. 14B sfGFP show absorbance scans of the native and alkaline denatured protein. FIG. 14C shows spectral scans as described in FIGS. 14A-14B of FOLD6, with absorbance collected every 1 min for 12 min. n=2, mean±s.d. FIGS. 14D-14K show time-dependent FP denaturation in 1 M NaOH (Methods). Absorbance at 447 nm (brown) and 505 nm (green) was measured every 10 s, promptly after mixing 2 M NaOH into a cuvette containing the same volume of FP solution (A505 c 1. 0.3-0.9) in phosphate buffered saline (PBS) for a final concentration of 1 M NaOH. The initial value at t=0 s is the absorbance of the same native protein stock solution when diluted separately 1:1 into PBS without NaOH. Curve fits for 505 nm absorbance data: monoexponential, mhYFP; double-exponential, mF4Y-SR, mF4P, FOLD6. The alkali-denatured avGFP-type chromophore (e=44.000 M-1 cm-1) shows maximal absorbance at −447 nm. The native absorbance maxima for all FPs in this figure are between 488-514 nm. Background subtracted absorbance values are shown on the y-axis without normalization. n=3, mean±s.d. For mGreenLantern and hfYFP, n=2.

FIG. 15 shows kinetic unfolding of V206 and K206 mutants in GdnHCI. Fluorescent proteins were unfolded in 6.3 M GdnHCI, pH 7.5. Asterisks indicate the final mutants, i.e., mGreenLantern, mF4Y-SR, hfYFP, and mhYFP, whose properties are reported in Table 2 and elsewhere.

FIGS. 16A-16H show rational engineering and characterization of fluorescent proteins using only 18 of the 20 naturally occurring amino acids. FIG. 16A shows location of Trp57 in a high-resolution eGFP crystal structure (PDB ID: 4EUL), with interactions of particular interest indicated by dashed lines. Showing: vdW interactions, including weakly polar interactions from C48; sulfur-aromatic interactions at approximately 5.5 A distance to the ring centroid; hydrogen bonds. FIG. 16B shows absorbance spectra of W57F mutants and parental templates. FIG. 16C shows isothermal melting of W57F mutants at 80° C. mF4P and its W57F derivative denature in a double exponential manner featuring a prolonged slow phase after −80-90% of the initial fluorescence is quenched. FIG. 16D shows kinetic unfolding in 3.6 M GdnSCN, pH 7.5, with Xex/Xem=495/525 nm. FIG. 16E shows melting curves of W57F mutants compared to the original proteins. FIG. 16F shows whole-cell fluorescence intensity of E. coli cultures grown overnight before extraction of soluble protein. One experiment is shown. FIG. 16G shows quantitation of Coomassie gels for −27 kDa band intensity, representing the FP monomer, as a measure of soluble protein yield. n=3 replicate experiments, mean±s.d. FIG. 16H shows spectroscopic characterization table of W57F mutants. mF4P-W57F and hfYFP-W57F contain no cysteine or tryptophan residues. Molecular brightness=x 0103. E. coli brightness: fluorescence data from f with experimental samples normalized to eGFP.

FIGS. 17A-17D show proton wires of hyperfolder YFPs and FOLD6. FIG. 17A shows hfYFP and mhYFP superposition. mhYFP displays an atypical E222 conformation for a YFP in which E222 is hydrogen-bonded (H-bonded) to [N2] and 5205 instead of [N2] and wat². FIG. 17B shows FOLD6 and Clover superposition. In Clover, E222 is H-bonded to [N2] but not to S205, and this is the only E222 conformation detected. The FOLD6 E222 is H-bonded to [N2] and to wat₃, completing a proton wire to the chromophore phenolate. Watt and wat₂ are highly conserved structural water molecules in avFPs, whereas wat₃ is rarely observed. [N2]: nitrogen atom of the chromophore imidazolinone ring. FIG. 17C shows overhead view of hfYFP and Citrine crystal structures. The chromophore phenolate points toward [3-strand 7 at the 12 o'clock position. The polar and nonpolar cores of the protein are indicated at the 7 o'clock and 3 o'clock positions. The F46L and F64L mutations in hfYFP, relative to Citrine, are located at the 3 o'clock position (bolded). The hfYFP H-bond network, identical to Citrine's, is shown. FIG. 17D shows FOLD6 cutaway side view of the protein's polar core, with the central a-helix visible, and the chromophore phenolate pointing toward B-strand 7. H2O3 is stacked on top. sfGFP side chains of H169 and 1167 are visible in grey line form for comparison. Dashes: H-bonds.

FIGS. 18A-18D show chromophore conservation and proposed role of C48 and C70 in avFPs. FIG. 18A shows superposition of hfYFP, eYFP, Citrine, and Venus. These four FPs share the same general chromophore H-bond network and side-chain orientations, although H-bond distances may vary up to −0.5 A. The same structural water molecules present in most avFPs, which we refer to as watt and wat₂, are observed here. FIG. 18B shows structure of the hfYFP chromophore environment as seen from B-strand 8. Y203 is pictured above the chromophore. Dashes: hfYFP H-bond network, sulfur-aromatic interactions from M69 with interaction distances averaging −6 A, which would stabilize the chromophore (CRO), Y203, and especially the F84 side chain to which the sulfur atom shows an orientational preference. This configuration improves hydrophobic packing relative to the wild-type Q69 residue. FIG. 18C shows the C48S mutation in hfYFP produces a tight 2.3 A H-bond between the S48 hydroxyl side chain and the G51 carbonyl. L53 has rotated relative to its conformation in eGFP to stabilize W57 through vdW forces. In eGFP, C48 of eGFP is just close enough to the electropositive edges of the F27 and W57 rings, with an average 5.5 A distance. Note that W57 No is Hbonded to D216 0 61 in every structure, but we have removed the foreground D216 side-chain in the images to improve visualization of residue 48. FIG. 18D shows the C70V mutation in hfYFP eliminates the lone-pair electrons of the C70 sulfur atom that in eYFP and other avFPs, can interact with the electropositive edges of the F8, F71, and Y92 ring to stabilize them (dashes: sulfuraromatic interactions). These sulfur-aromatic interactions likely provide greater support than vdW forces alone. Consequently, ring positions have shifted 0.3-0.5 A in hfYFP relative to Venus to adjust to new vdW distances from V70.

FIGS. 19A-19E show example of stabilizing salt bridges in avFPs. FPs were aligned in PyMOL and are presented in the same orientation. Dashed line: salt bridge <4 A distance, H-bond 3.0 A. FIG. 19A shows eGFP displays an H-bond between E17 and S30 in addition to the E115-R122 salt bridge observed in each of the depicted structures. FIG. 19B shows sfGFP. FIG. 19C shows FOLD6, with multiple conformations observed for the critical R30 superfolder mutation that stabilizes several neighboring side-chains. FIG. 19D shows in hfYFP, R30 is found in a single conformation that secures it with four ionic bonds, one each to the E17 and D19 carboxylates, and two to main-chain carbonyls. FIG. 19E shows mhYFP shows a sfGFP-like salt bridge network with an additional R30 conformation observed.

FIGS. 20A-20F show library generation and screening of GFPs with a large Stokes shift. FIG. 20A shows excitation spectrum of hfYFP and hfYFP-KSI (hfYFP-V206K/G65S/Y2031). The G65S/Y2031 mutations produced a 405 nm excitable band and an −50 nm hypsochromic shift of the B-band. V206K does not affect the spectrum. FIG. 20B shows degenerate codon sets chosen to mutate hfYFP-KSI at residues shown in FIG. 5a-b to produce an LSS FP without B-band excitation. FIG. 20C shows excitation spectra of 33 mutants from the library in FIG. 20B that were selected from LB-agar plates based on high 410-nm and low 470-nm excitation by eye under LED illumination. Light green: LSSA12. Inset: zoom of the B-band excitation range. FIG. 20D shows kinetic unfolding of clarified lysate of 23 mutants from library FIG. 20C in buffered 6.3 M GdnHCI. FIG. 20E shows kinetic unfolding of purified FPs. FIG. 20F shows excitation spectra of LSSA12 point mutants. The spectra of these mutants reinforce the importance of residues S65 and D222 for eliminating B-band excitation of LSSA12. Note that the excitation spectra of mT-Sapphire and LSSA12-S65G overlap, as do LSSA12 and LSSA12-E204D.

FIGS. 21A-21K show Additional characterization of LSSmGFP. FIG. 21A shows introducing the V68Q mutation into mT-Sapphire greatly diminished the 488 nm excitation band, suggesting generalizability of this LSSmGFP derived mutation for spectral tuning of LSS FPs. FIG. 21B shows melting curve derivative plots generated from the thermofluor assay. Here, SYPRO Orange is used instead of endogenous fluorescence. Tm values are reported in Table 5. FIG. 21C shows kinetic unfolding of purified FPs at 1 uM concentration in buffered GdnHCI 6.3 M solution, pH 7.4. FIG. 21D shows purified FP at 0.1 uM concentration incubated at RT in 16 different solutions of H₂O₂ in Tris buffer, pH 7.4, for exactly 15 min. All data points are shown and fit to a mono-exponential decay equation. n=3, mean±s.d. FIG. 21E shows representative images of LSS FPs in the OSER assay when expressed from pCytERM fusions. White wedge in the second mT-Sapphire image indicates a highly stressed cell with sheeted ER that was rarely seen in other FPs but was commonly encountered in mT-Sapphire. These cells were excluded from quantitation but might represent an important effect. Scale bars, 25 pm. FIG. 21F shows OSER assay results analyzed using scoring criteria described in FIG. 12. FIGS. 21G-21K shows live HeLa cells imaged after overnight transfection using plasmids encoding: FIG. 21G, LifeAct-7aa-LSSmGFP; actin. FIG. 21H, LSSmGFP-6aa-tubulin; tubulin. FIG. 21IG. LSSmGFP-15aa-clathrin; clathrin. FIG. 21J, COX8A[x4]-4aa-LSSmGFP; mitochondria. FIG. 21K pCytERM-LSSmGFP; endoplasmic reticulum. Scale bars: 10 um.

FIG. 22 shows benchtop fluorescence-assisted protein purification workflow. As described in Methods, proteins were purified by Ni-NTA chromatography (immobilized metal affinity chromatography, or IMAC) under fully denaturing conditions with all buffers containing 6 M GdnHCI. If cleavage of the fusion protein is unimportant for downstream assays, the experiment is completed once the fusion protein is dialyzed out of denaturing purification buffer. Otherwise, the fusion construct illustrated in FIG. 5G is cleaved using TEV protease and the protein of interest (P01) is isolated using IMAC: His6-TEV and His6-hfYFP are adsorbed onto the resin while the untagged and cleaved POI elutes in native buffer of choice for collection. For fluorescence-assisted purification under native rather than denaturing conditions: the soluble fraction is purified by IMAC; fusion is cleaved using TEV protease in a dialysis cassette (or after dialysis); and the unbound, cleaved POI is obtained by Ni-NTA chromatography in the flow-through while His6-TEV and His6-hfYFP remain bound to the resin. All steps of purification can be visualized using 470 nm illumination for the hfYFP fusion or 405 nm illumination for the LSSmGFP fusion. The final image (“Isolated POI”) depicts successful isolation of mScarlet from the hfYFP-mScarlet fusion in phosphate buffered saline after cleavage. *=if the refolding buffer inhibits TEV protease activity, a separate dialysis step is recommended to improve activity. Similarly, if the TEV protease buffer selected is incompatible with IMAC (e.g., if nontrivial amounts of DTT or EDTA are present), then a dialysis step should be performed after cleavage, before IMAC.

FIG. 23 shows that mhYFP is compatible with commercially available antibodies designed for eGFP. Antibody signal co-localized with HEK293T cells expressing cytosolic FPs. mF4Y-SR is a hyperfolder mutant related to mGreenLantern. mNeonGreen is derived from from B. lanceolatum rather than A. victoria and therefore serves as a negative control. Antibody channel: Alexa 555 conjugated Donkey secondary antibody. Merged image features DAPI in the blue channel. mGreenLantern was shown to react with the same set of antibodies used here in Campbell et al., PNAS, 2020. Scale bars, 25 um.

FIG. 24 shows statistics for FIG. 3 and FIG. 21. Data were analyzed using one-way ANOVA with multiple comparisons. Full details of the experiment from FIG. 3 and FIG. 21 are available in Methods.

FIG. 25 shows fluorescent protein amino acid sequence comparison to mGreenLantern (mGL). Mutations that are most important to the properties of the specific FP are highlighted. The alignment is bolded when a residue has changed in any FP relative to mGL at the indicated amino acid position. Note: FP engineers traditionally append the C-terminal amino acids “ . . . GMDELYK*” (asterisk for stop codon) to most FPs, if they are not already present. The FPs shown here instead use C-terminal sequence “ . . . DMDELYK*” (G232D mutation) for mGL, and “ . . . DMNELYK*” (G232D/D234N mutations) for hfYFP, mhYFP, LSSA12, and LSSmGFP. These mutations are important for the stability properties and brightness of these proteins. For optimal performance (and properties as characterized in this manuscript), we recommend preserving the complete C-terminal sequences.

FIGS. 26A-26B show protein quantified from expression of fusion constructs in E. coli. FIG. 26A shows coomassie gels of soluble protein, insoluble protein, and protein from the media of the same cultures (without cells). Equal quantity of protein was run in each lane as determined by BCA assay, except for the media condition where equal volume was used without adjustment. The molecular weight (MW) predicted by ExPASy for the FP fusions to mScarlet-1 (mSca). Bacillus circulars xylanase (Bcx), and streptavidin (SAV) are approximately 57 kDa, 51 kDa, and 44 kDa, respectively. The MW of an avFP is −27 kDa. FIG. 26B shows quantification of the protein fusion hands from gels in a using standard ImageJ densitometry tools. Numbers above the bar graph are the calculated ratios of soluble protein to insoluble protein from raw densitometry values. n=3, mean±s.d.

### DETAILED DESCRIPTION

The disclosure provides fluorescent proteins comprising the amino acid sequence selected from SEQ ID NOs: 1, 3, 5, 7-10, and 12.

The disclosure provides the rational engineering of a remarkably stable yellow fluorescent protein, ‘hyperfolder YFP,’ (hfYFP) that withstands chaotropic conditions that denature most biological structures within seconds, including superfolder GFP. hfYFP contains no cysteines, is chloride insensitive, and tolerates aldehyde and osmium tetroxide fixation better than common fluorescent proteins (FPs), enabling its use in protein-retention expansion microscopy (proExM) and correlative light and electron microscopy (CLEM). We solved crystal structures of hfYFP (to 1.6 Å resolution), a monomeric variant (Monomeric hyperfolder YFP, mhYFP) (1.7 Å), and an mGreenLantern mutant (1.2 Å), and then rationally engineered highly stable 405 nm excitable green FPs, LSSmGFP and LSSA12, from these structures. Lastly, we directly exploited the chemical stability of hfYFP and LSSmGFP by devising a fluorescence-assisted protein purification strategy enabling all steps of denaturing Ni-NTA chromatography to be visualized using ultraviolet or blue light. hfYFP and LSSmGFP represent a new generation of robustly stable FPs developed for advanced biotechnological applications.

Recently developed green-to-red photoconvertible FPs, mEos4a and mEos4b, showed impressive resistance to the quenching effects of aldehyde fixation and osmium tetroxide (OsO₄). Diverse FPs—particularly constitutively fluorescent ones like green fluorescent protein (GFP)—are required to expand the reach of such methods. Moreover, in addition to requirements of specialized modalities like CLEM, FPs should have favorable properties for routine use, including fast and complete folding and maturation, high brightness and photostability, and low oligomericity when used in fusions.

We developed mGreenLantern a bright, monomeric green FP that is well suited for ExM and tissue clearing methods such as 3DISCO, thereby facilitating neuronal imaging experiments including the tracing of supraspinal projections in a cleared, fully intact brain, while bypassing the laborious and time-consuming antibody enhancement steps. The greater resistance of mGreenLantern to chemical and thermal denaturation compared to other common FPs such as eGFP, mClover3 (Bajar et al., 2016), and mNeonGreen (Shaner et al., 2013), prompted us to expand our study of mGreenLantern to realize additional improvements to protein stability and thereby enhance functionality in advanced imaging modalities.

Here we generated well-folded cysteine-free FPs from mGreenLantern with stability characteristics eclipsing those of the renowned superfolder GFP (sfGFP). Our structure-guided screening efforts produced a yellow fluorescent protein, ‘hyperfolder YFP’ (hfYFP), whose stability in notoriously chaotropic conditions, including OsO₄, allowed it to survive CLEM sample preparation with fluorescence retention matching mEos4b's. hfYFP retained greater fluorescence after ExM and aldehyde fixation than eGFP, eYFP, mClover3, and mNeonGreen.

We solved the crystal structures of hfYFP (to 1.7 Å resolution), a monomeric variant, mhYFP (1.6 Å), and a cysteine-free mGreenLantern variant called FOLD6 (1.2 Å), shedding light on structure-function relationships underpinning their brightness and stability. By applying this knowledge, we generated fluorescent proteins LSSA12 and LSSmGFP that are exclusively excited by 405 nm excitation, in contrast to mT-Sapphire, which is inconveniently co-excited with eGFP under 488 nm illumination. We then leveraged the high stability of hfYFP and LSSmGFP by developing a simple fluorescence-assisted protein purification strategy using hfYFP or LSSmGFP fusions to visualize all steps of native and even denaturing Ni-NTA chromatography (6 M guanidinium hydrochloride) using inexpensive ultraviolet or blue LEDs at the benchtop.

With its remarkable resilience, high solubility, low oligomerization propensity, chloride insensitivity, and lack of cysteine residues, hfYFP is a versatile FP that overcomes most of eYFP's limitations and can directly replace it in many applications. hfYFP offers powerful cross-compatibility with proExM and CLEM, as well as applications traditionally served by sfGFP. Our crystal structures offer launch points for engineering novel biosensors and may even provide simple, actionable solutions to folding problems of existing sensors. Hyperfolder YFP is ripe for biotechnological applications that have previously been unreachable.

In this study, we have described the engineering, extensive characterization, and application of unusually durable fluorescent proteins in stability assays; in expansion microscopy (proExM); in the aldehyde fixation, osmication, embedding, sectioning, and imaging steps of correlative light and electron microscopy (CLEM); and in fluorescence-assisted protein purification. Through structure-guided engineering and screening based on GdnHCI unfolding rates and melting temperature (Tₘ) alongside rigorous spectroscopic characterization, we produced a series of avFP mutants demonstrating superior stability over sfGFP in every assay that we performed. hfYFP survived all steps of CLEM preparation and may find use in super-resolution imaging and other advanced microscopy methods, as well as numerous biotechnological applications that can benefit from a fluorophore whose melting temperature is only 6° C. below the boiling point of water.

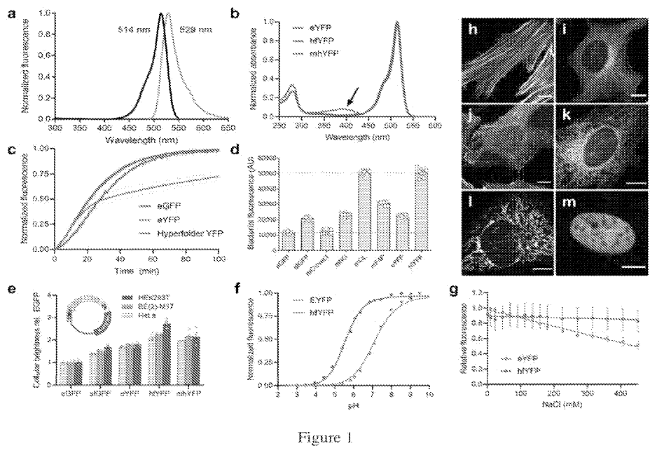

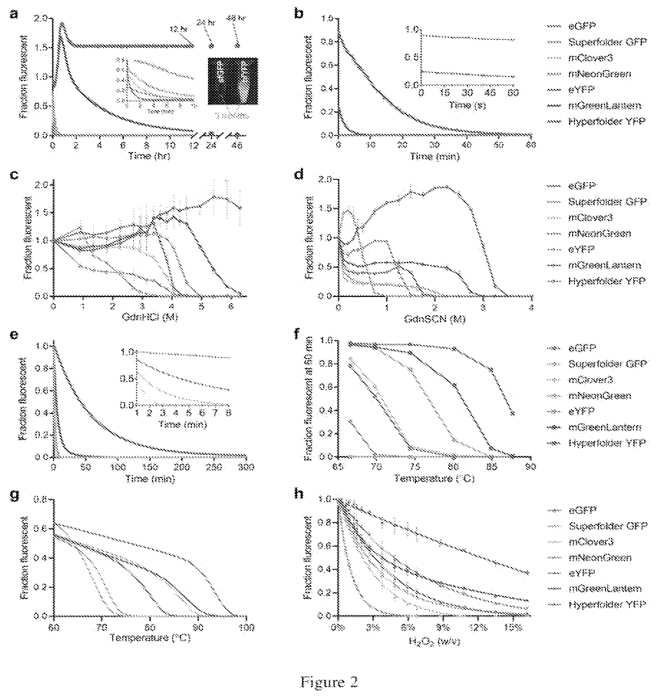

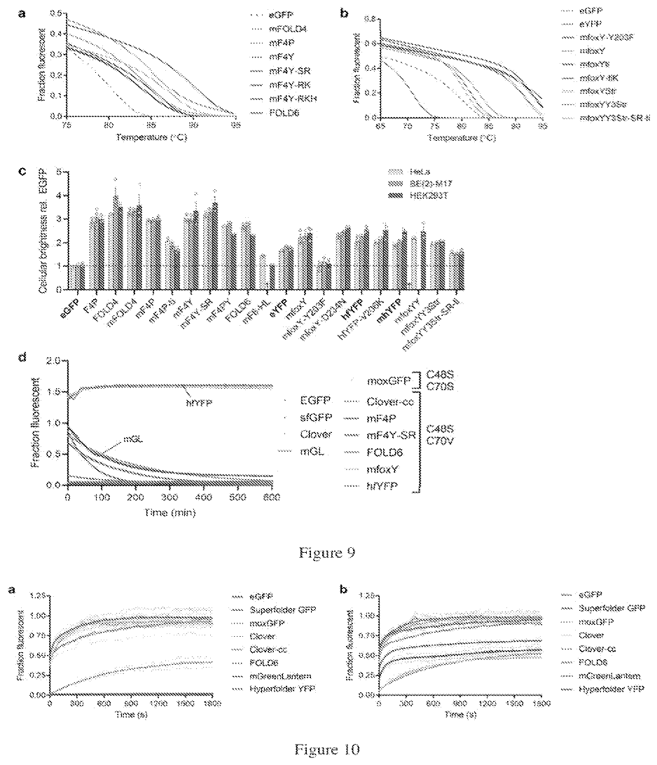

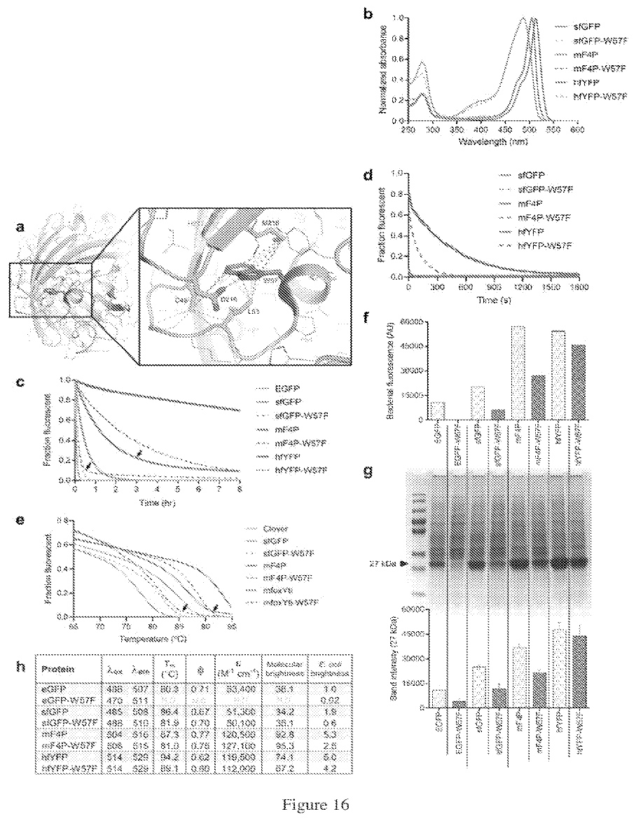

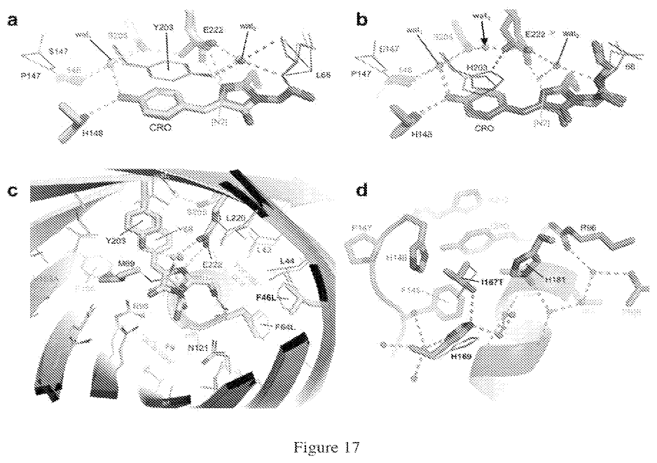

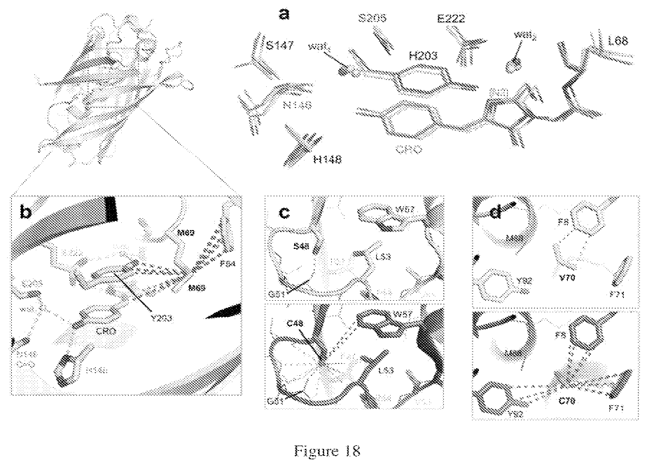

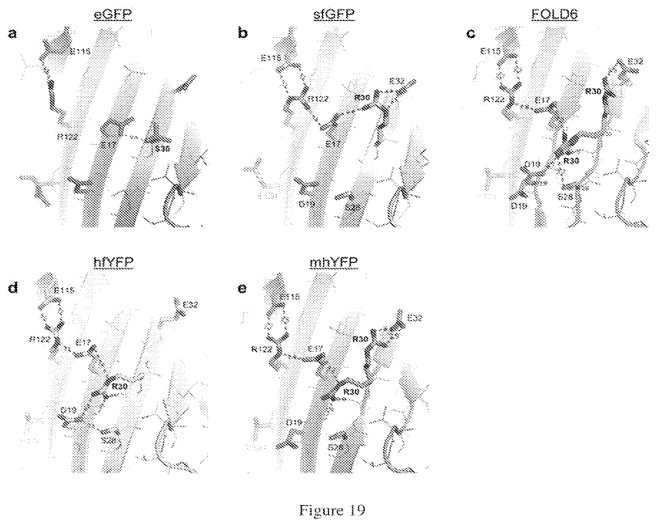

In addition to the CLEM application, we achieved our sub-aims of advancing and better understanding the stability of mGreenLantern (FIG. 2): we eliminated cysteines without disrupting fluorescence (FIG. 7); ensured no 405 nm excitability (FIG. 1B); ensured that the brightness of human cells expressing the FPs would be no less than those expressing eGFP or eYFP (FIG. 1E; FIG. 9C); described structure-function correlates of the thermodynamic stability (FIG. 17; FIG. 18); and demonstrated that the “hyperfolder” FPs can tolerate unusual substitutions, even to the highly conserved C48, C70, and W57 residues. The hfYFP-W57F mutant, with an 18-amino acid generic code entirely lacking the stabilizing Trp and Cys residues that are conserved across all avFPs, was brighter and more stable than even wild-type sfGFP (FIG. 16). Benefiting from the hfYFP crystal structure, we eliminated hfYFP's 514 nm excitability and produced two exclusively 405 nm excitable GFPs with a large Stokes shift (LSS), LSSA12 and LSSmGFP, thereby overcoming the cross-excitation problem of mT-Sapphire (FIGS. 5C-5E) while sacrificing little molecular brightness (Table 5) and enhancing chemical thermodynamic stability (FIGS. 21B-21D). LSSmGFP has high chemical and thermodynamic stability and the same molecular brightness as mAmetrine, while lasting twice as long under laser-scanning confocal illumination before photobleaching.

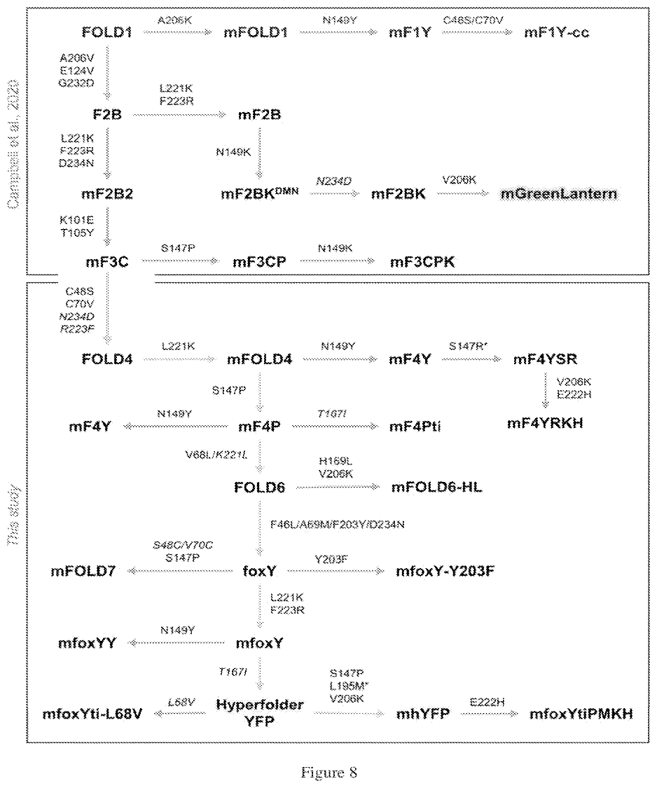

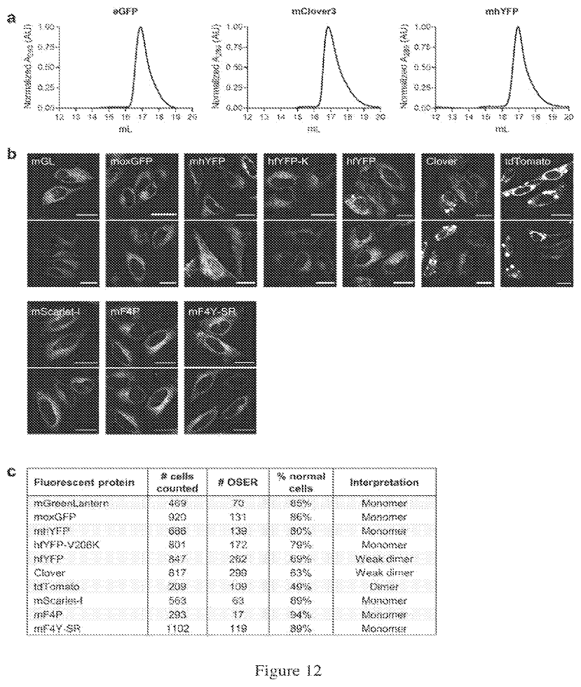

Our data confirm that misfolding and lower soluble protein yield (FIG. 11; FIG. 16G) are principally responsible for the diminished brightness of cysteine- and tryptophan-substituted avFPs, and that the brightness and stability deficits incurred by such radical structural perturbations can largely be corrected without modifying the spectral properties (FIG. 16H). Using structure-guided engineering, we conferred the sodium hydroxide (NaOH) resistance that we first identified in FOLD6 into hfYFP (FIG. 14) and enhanced hfYFP's monomericity (FIG. 12) in a single step (FIG. 8), producing monomeric hyperfolder YFP (mhYFP) whose spectral properties were identical to hfYFP's (Table 2). Of all the mutants and the handful of bright avFPs that we compared, hfYFP showed the greatest structural plasticity, implying that it will tolerate random mutagenesis and circular permutation at least as well as sfGFP. Indeed, hfYFP proved to be an excellent template for engineering two new LSS-FPs, LSSA12 and LSSmGFP. hfYFP performs well in fusions, and mhYFP offers a slightly more monomeric option at a very small cost to stability.

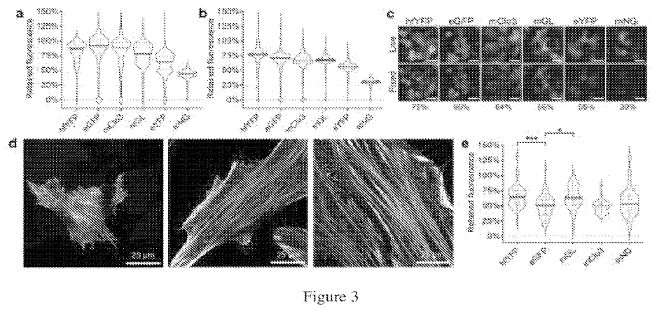

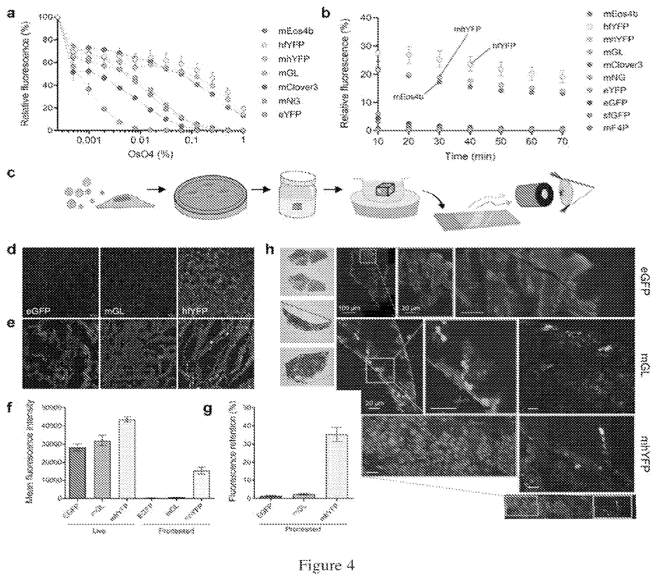

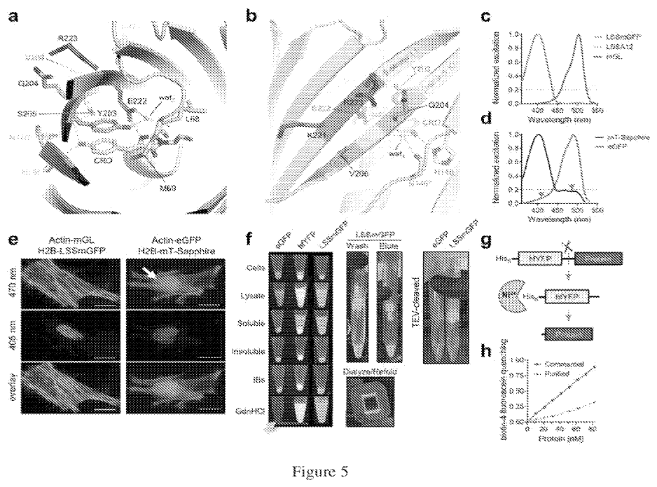

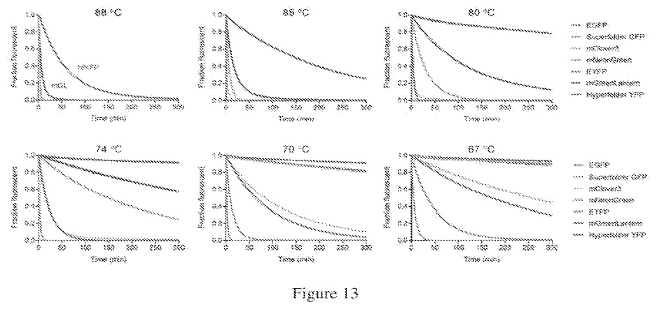

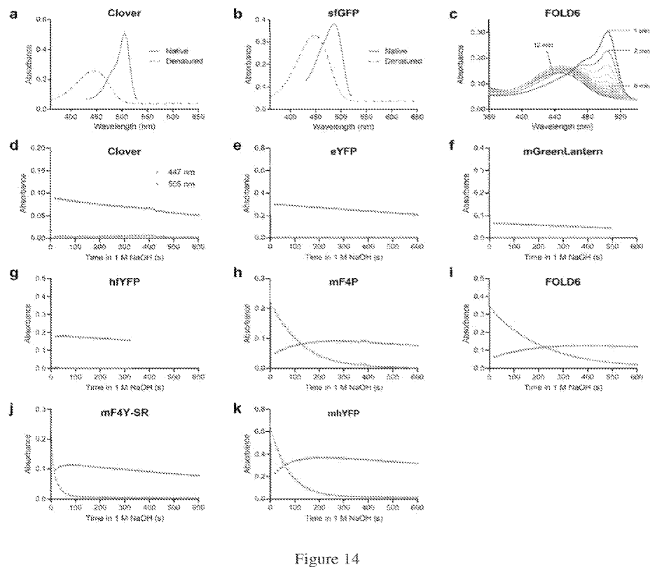

Hyperfolder YFP's stability (Table 3) is uncommon for any class of protein, and perhaps most notable is its peculiar stability in GdnHCl solutions of 7 M concentration (FIG. 2C), practically indefinitely (FIG. 2A). hfYFP's resilience was not an idiosyncratic response to guanidinium: apart from the GdnHCI and GdnSCN kinetic- and equilibrium-unfolding experiments, hfYFP retained more fluorescence than eGFP, sfGFP, mClover3, mNeonGreen, eYFP, and even mGL, at higher temperatures and for greater lengths of time (FIG. 13), in the presence of hydrogen peroxide (H₂O₂) (FIG. 2H), after exposure to paraformaldehyde (PFA) (FIG. 3D), PFA/glutaraldehyde (FIGS. 3B-3C), and in a 3% glyoxal/20% ethanol, pH 4.0 solution (FIGS. 3E-3F), and even osmium tetroxide (OsO₄) (FIGS. 4A-4B). The relatively high acid stability (pKₐ 5.6) and very high chloride resistance mean that hfYFP can be deployed in more organelles and other cellular environments without fear of artifacts. We found mNeonGreen to be by far the least stable FP we tested against temperature, guanidinium, H₂O₂, glutaraldehyde, and OsO₄—this FP should be used in challenging applications with great caution.

Even small gains in fluorescence retained after chemical fixation or other quenching processes can amplify cellular brightness differences, and vice versa. mGreenLantern, with its 6.0-fold greater fluorescence than eGFP in mammalian cells compared to 2.4-fold for hfYFP, may be best suited for proExM (FIG. 3A), while hfYFP may be most advantageous for CLEM due to its tolerance of osmication that matches that of mEos4b (FIG. 4A-4B). There might not be a single FP that can suit every possible application, but hfYFP has proven itself to be a versatile tool for routine imaging and shows promise in the tested super-resolution modalities.

The high-resolution crystal structures of hfYFP, mhYFP, and FOLD6 that we solved to 1.7 Å, 1.6 Å, and 1.2 Å resolution, respectively, offer excellent templates for biosensor engineering, particularly when combined with our characterization data (Tables 1-3), library development approach for the GFPs/YFPs, LSS-FPs, and the structural interpretations. Altogether, the crystallographic data and our characterized mutants enable us to describe which mutations are critical to hfYFP's function and should be preserved during engineering, which are expendable, which can be transferred into existing templates to enhance them, and how that may be accomplished.

We suggest that the dense hydrophobic packing of the hfYFP chromophore environment, in addition to surface mutations and interactions that stabilize the barrel structure, may help the protein resist core solvation effects that have been proposed as part of a two-stage mechanism for GdnHCl-induced protein denaturation, thereby extending the duration of fluorescence perhaps even while the protein is in a molten globule state. Knowledge of the biophysical nature of hfYFP's stability could be enhanced by experiments based on the unfolding data and crystal structures to calculate the free energy of unfolding, among other measurements, with the goal of revealing deeper structure-activity correlates including regional and whole-protein solvent-accessible surface area, protein volume, and surface net charge, with comparisons to eYFP, Citrine, and Venus. Molecular dynamics simulations could further clarify the nature of the GdnHCl and NaOH resistance and possibly reveal generalizable mechanisms that could in theory be transferred to other FPs beyond hfYFP—potentially even very sequence-diverse ones. Additional value remains to be unlocked from the hfYFP, mhYFP, and FOLD6 crystal structures.

Hyperfolder YFP and FOLD6 have potential to serve similar functions as sfGFP, such as multiple epitope tag insertion, extremophile research, reconstitution of split fragments, sensor stabilization, circular permutation, and random mutagenesis, while their stability improvements open doors to new applications that have not yet been realized for expression-enhanced biosensors, perhaps along with concomitant decreases in cytotoxicity due to the large improvements in protein folding and solubility.

hfYFP is a versatile protein that may find use in expansion microscopy (proExM), correlative light and electron microscopy (CLEM), and tissue clearing. Besides those applications, hfYFP and LSSmGFP enable fluorescence-assisted protein purification and may even act as visualizable solubility tags (FIGS. 5F-5G). Likewise, LSSmGFP and LSSA12 performed well in assays in which hfYFP excelled, including protein purification, and they may find similar uses. Biotechnological applications that were previously complicated or irresolvable due to superfolder GFP's limitations, such as the presence of cysteines and susceptibility to chemical denaturation, may now be in reach.

### Definitions

Unless defined otherwise, all technical and scientific terms used herein have the same meaning as commonly understood by one of ordinary skill in the art to which this disclosure is related. The headings provided herein are not limitations of the various embodiments, which can be had by reference to the specification as a whole. Moreover, the terms defined immediately below are more fully defined by reference to the specification in its entirety.

The terms “a,” “an” and “the” include plural referents unless the context in which the term is used clearly dictates otherwise. The terms “a” (or “an”), as well as the terms “one or more,” and “at least one” can be used interchangeably herein. Furthermore, “and/or” where used herein is to be taken as specific disclosure of each of the two or more specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

The term “comprise” is generally used in the sense of include, that is to say permitting the presence of one or more features or components. Wherever embodiments, are described herein with the language “comprising,” otherwise analogous embodiments described in terms of “consisting of,” and/or “consisting essentially of” are also provided.

The terms “about” and “approximately” as used in connection with a numerical value throughout the specification and the claims denotes an interval of accuracy, familiar and acceptable to a person skilled in the art. In general, such interval of accuracy is ±10%. Alternatively, and particularly in biological systems, the terms “about” and “approximately” may mean values that are within an order of magnitude, preferably ≤5-fold and more preferably ≤2-fold of a given value.

The term “fusion protein” refers to a protein composed of two or more polypeptides that, although typically unjoined in their native state, are joined to form a single continuous polypeptide. It is understood that the two or more polypeptide components can either be directly joined or indirectly joined through a sequence of one or more amino acids which acts as a spacer or a linker.

The term “linkage” or “linker” (L) is used herein to refer to an atom or a collection of atoms used to link, preferably by one or more covalent bonds, interconnecting moieties such as two polymer segments or a terminus of a polymer and a reactive functional group present on a bioactive agent, such as a polypeptide.

A protein, polynucleotide, vector, cell, or composition which is “isolated” is a protein (e.g., antibody), polynucleotide, vector, cell, or composition which is in a form not found in nature. Isolated proteins, polynucleotides, vectors, cells or compositions include those which have been purified to a degree that they are no longer in a form in which they are found in nature. In some embodiments, a protein, polynucleotide, vector, cell, or composition which is isolated is substantially pure. Isolated proteins and isolated nucleic acid will be free or substantially free of material with which they are naturally associated such as other polypeptides or nucleic acids with which they are found in their natural environment, or the environment in which they are prepared (e.g., cell culture) when such preparation is by recombinant DNA technology practiced in vitro or in vivo. Proteins and nucleic acid may be formulated with diluents or adjuvants and still for practical purposes be isolated—for example the proteins will normally be mixed with gelatin or other carriers if used to coat microtitre plates for use in immunoassays, or will be mixed with pharmaceutically acceptable carriers or diluents when used in diagnosis or therapy.

The terms “polynucleotide” and “nucleic acid” are used interchangeably and are intended to encompass a singular nucleic acid as well as plural nucleic acids, and refers to an isolated nucleic acid molecule or construct, e.g., messenger RNA (mRNA), complementary DNA (cDNA), or plasmid DNA (pDNA). In certain embodiments, a polynucleotide comprises a conventional phosphodiester bond or a non-conventional bond (e.g., an amide bond, such as found in peptide nucleic acids (PNA)). The term “nucleic acid” refers to any one or more nucleic acid segments, e.g., DNA, cDNA, or RNA fragments, present in a polynucleotide.

The terms “polypeptide,” “peptide,” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

A “recombinant” polypeptide, protein or antibody refers to polypeptide, protein or antibody produced via recombinant DNA technology. Recombinantly produced polypeptides, proteins and antibodies expressed in host cells are considered isolated for the purpose of the present disclosure, as are native or recombinant polypeptides which have been separated, fractionated, or partially or substantially purified by any suitable technique.

The term “percent sequence identity” or “percent identity” between two polynucleotide or polypeptide sequences refers to the number of identical matched positions shared by the sequences over a comparison window, taking into account additions or deletions (i.e., gaps) that must be introduced for optimal alignment of the two sequences. A matched position is any position where an identical nucleotide or amino acid is presented in both the target and reference sequence. Gaps presented in the target sequence are not counted since gaps are not nucleotides or amino acids. Likewise, gaps presented in the reference sequence are not counted since target sequence nucleotides or amino acids are counted, not nucleotides or amino acids from the reference sequence. The percentage of sequence identity is calculated by determining the number of positions at which the identical amino-acid residue or nucleic acid base occurs in both sequences to yield the number of matched positions, dividing the number of matched positions by the total number of positions in the window of comparison and multiplying the result by 100 to yield the percentage of sequence identity. The comparison of sequences and determination of percent sequence identity between two sequences can be accomplished using readily available software programs. Suitable software programs are available from various sources, and for alignment of both protein and nucleotide sequences. One suitable program to determine percent sequence identity is bl2seq, part of the BLAST suite of program available from the U.S. government's National Center for Biotechnology Information BLAST web site (blast.ncbi.nlm.nih.gov). Bl2seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at www.ebi.ac.uk/Tools/psa.

Reference to a nucleotide sequence “encoding” a polypeptide means that the sequence, upon transcription and translation of mRNA, produces the polypeptide. This includes both the coding strand, whose nucleotide sequence is identical to mRNA and whose sequence is usually provided in the sequence listing, as well as its complementary strand, which is used as the template for transcription. As any person skilled in the art recognizes, this also includes all degenerate nucleotide sequences encoding the same amino acid sequence. Nucleotide sequences encoding a polypeptide include sequences containing introns.

### Fluorescent Proteins

Hyperfolder YFP (hfYFP) withstands chaotropic conditions that denature most biological structures within seconds, including superfolder GFP. hfYFP contains no cysteines, is chloride insensitive, and tolerates aldehyde and osmium tetroxide fixation better than common FPs, enabling its use in protein-retention expansion microscopy (proExM) and correlative light and electron microscopy (CLEM). We solved crystal structures of hfYFP (to 1.6 A resolution), a monomeric variant, mhYFP (1.7 Å), and an mGreenLantern mutant (1.2 Å), and then rationally engineered highly stable 405 nm excitable green FPs, LSSmGFP and LSSA12, from these structures. Lastly, we directly exploited the chemical stability of hfYFP and LSSmGFP by devising a fluorescence-assisted protein purification strategy enabling all steps of denaturing Ni-NTA chromatography to be visualized using ultraviolet or blue light. hfYFP and LSSmGFP represent a new generation of robustly stable FPs developed for advanced biotechnological applications.

Hyperfolder YFP (hfYFP)

The amino acid sequence of hyperfolder YFP (hfYFP) is as follows:

| (SEQ ID NO: 1) | |
|---|---|
| 1 | MVSKGEELFT GVVPILVELD GDVNGHKESV RGEGEGDATN GKLTLKLIST TGKLPVPWPT |
| 61 | LVTTLGYGLM VFARYPDHMK QHDFFKSAMP EGYVQERTIS FEDDGYYKTR AEVKFEGDIL |
| 121 | VNRIVERGID FKEDGNILGH KLEYNFNSHN VYITADKQKN GIKANFKIRA NVEDGGVQLA |
| 181 | DHYQQNTPIG DGPVLLPDNH YLSYQSVLSK DPNEKRDHMV LKERVTAAGI THDMNELYK |

The nucleic acid sequence of hyperfolder YFP (hfYFP) is as follows:

| (SEQ ID NO: 2) | |
|---|---|
| 1 | ATGGTGAGCA AGGGCGAGGA GCTGTTCACC GGGGTGGTGC CCATCCTGGT CGAGCTGGAC |
| 61 | GGCGACGTAA ACGGCCACAA GTTCAGCGTC CGCGGCGAGG GCGAGGGCGA TGCCACCAAC |
| 121 | GGCAAGCTGA CCCTGAAGCT CATCTCCACC ACCGGCAAGC TGCCCGTGCC CTGGCCCACC |
| 181 | CTCGTGACCA CCTTAGGCTA CGGCCTGATG GTGTTCGCCC GCTACCCCGA CCACATGAAG |
| 241 | CAGCACGACT TCTTCAAGTC CGCCATGCCC GAAGGCTACG TCCAGGAGCG CACCATCTCT |
| 301 | TTCGAGGACG ACGGTTACTA CAAGACCCGC GCCGAGGTGA AGTTCGAGGG CGACACCCTG |
| 361 | GTGAACCGCA TCGTGCTGAA GGGCATCGAC TTCAAGGAGG ACGGCAACAT CCTGGGGCAC |
| 421 | AAGCTGGAGT ACAACTTCAA CAGCCACAAC GTCTATATCA CGGCCGACAA GCAGAAGAAC |
| 481 | GGCATCAAGG CTAACTTCAA GATCCGCCAC AACGTTGAGG ACGGCGGCGT GCAGCTCGCC |
| 541 | GACCACTACC AGCAGAACAC CCCCATCGGC GACGGCCCCG TGCTGCTGCC CGACAACCAC |
| 601 | TACCTGAGCT ACCAGTCCGT CCTGAGCAAA GACCCCAACG AGAAGCGCGA TCACATGGTC |
| 661 | CTGAAGGAGA GGGTGACCGC CGCCGGGATT ACACATGACA TGAACGAGCT GTACAAG |

Monomeric Hyperfolder YFP (mhYFP)

The amino acid sequence of mhYFP is as follows:

| (SEQ ID NO: 3) | |
|---|---|
| 1 | MVSKGEELFT GVVPILVELD GDVNGHKFSV RGEGEGDATN GKLTLKLIST TGKLPVPWPT |
| 61 | LVTTLGYGLM VFARYPDHMK QHDFFKSAMP EGYVQERTIS FEDDGYYKTR AEVKFEGDIL |
| 121 | VNRIVLKGID FKEDGNILGH KLEYNENPHN VYITADKQKN GIKANFKIRH NVEDGGVQLA |
| 181 | DHYQQNTPIG DGPVLMPDNH YLSYQSKLSK DPNEKRDHMV LKERVTAAGI THDMNELYK |

The nucleic acid sequence of mhYFP is as follows:

| (SEQ ID NO: 4) | |
|---|---|
| 1 | ATGGTGAGCA AGGGCGAGGA GCTGTTCACC GGGGTGGTGC CCATCCTGGT CGAGCTGGAC |
| 61 | GGCGACGTAA ACGGCCACAA GTTCAGCGTC CGCGGCGAGG GCGAGGGCGA TGCCACCAAC |
| 121 | GGCAAGCTGA CCCTGAAGCT CATCTCCACC ACCGGCAAGC TGCCCGTGCC CTGGCCCACC |
| 181 | CTCGTGACCA CCTTAGGCTA CGGCCTGATG GTGTTCGCCC GCTACCCCGA CCACATGAAG |
| 241 | CAGCACGACT TCTTCAAGTC CGCCATGCCC GAAGGCTACG TCCAGGAGCG CACCATCTCT |
| 301 | TTCGAGGACG ACGGTTACTA CAAGACCCGC GCCGAGGTGA AGTTCGAGGG CGACACCCTG |
| 361 | GTGAACCGCA TCGTGCTGAA GGGCATCGAC TTCAAGGAGG ACGGCAACAT CCTGGGGCAC |
| 421 | AAGCTGGAGT ACAACTTCAA CCCCCACAAC GTCTATATCA CGGCCGACAA GCAGAAGAAC |
| 481 | GGCATCAAGG CTAACTTCAA GATCCGCCAC AACGTTGAGG ACGGCGGCGT GCAGCTCGCC |
| 541 | GACCACTACC AGCAGAACAC CCCCATCGGC GACGGCCCCG TGCTGATGCC CGACAACCAC |
| 601 | TACCTGAGCT ACCAGTCCAA GCTGAGCAAA GACCCCAACG AGAAGCGCGA TCACATGGTC |
| 661 | CTGAAGGAGA GGGTGACCGC CGCCGGGATT ACACATGACA TGAACGAGCT GTACAAG |

### LSSmGFP

The amino acid sequence of LSSmGFP, which is hfYFP-T43S G65S/L68Q/H77N/K140N/Y203I/V206K, is as follows:

```
(SEQ ID NO: 5)
    1 MVSKGEELFT GVVPILVELD GDVNGHKESV RGEGEGDAIN GKLSLKLIST TGKLPVPWPT
  61 LVTILSYGQM VFARYPDNMK QHDFFKSAMP EGYVQERTIS FEDDGYYKTR AEVKFEGDTL
121 VNRIVLKGID FKEDGNILGH NLEYNFNSHN VYITADKQKN GIRANFKIRA NVEDGGVQLA
181 DHYQQNTPIG DGPVLLPDNH YLSIQSKLSK DPNEKRDHMV LKERVTAAGI THDMNELYK
```

The nucleic acid sequence of LSSmGFP is as follows:

| (SEQ ID NO: 6) | |
|---|---|
| 1 | ATGGTGAGCA AGGGCGAGGA GCTGTTCACC GGGGTGGTGC CCATCCTGGT CGAGCTGGAC |
| 61 | GGCGACGTAA ACGGCCACAA GTTCAGCGTC CGCGGCGAGG GCGAGGGCGA TGCCACCAAC |
| 121 | GGCAAACTGT CCCTGAAGCT CATCTCCACC ACCGGCAAGC TGCCCGTGCC CTGGCCCACC |
| 181 | CTCGTGACCA CCTTAAGCTA CGGCCAGATG GTGTTCGCCC GCTACCCTGA CAACATGAAG |
| 241 | CAGCACGACT TCTTCAAGTC CGCCATGCCC GAAGGCTACG TCCAGGAGCG CACCATCTCT |
| 301 | TTCGAGGACG ACGGTTACTA CAAGACCCGC GCCGAGGTGA AGTTCGAGGG CGACACCCTG |
| 361 | GTGAACCGCA TCGTGCTGAA GGGCATCGAC TTCAAGGAGG ACGGCAACAT CCTGGGGCAC |
| 421 | AACCTGGAGT ACAACTTCAA CAGCCACAAC GTCTATATCA CGGCCGACAA GCAGAAGAAC |
| 481 | GGCATCAAGG CTAACTTCAA GATCCGCCAC AACGTTGAGG ACGGCGGCGT GCAGCTCGCC |
| 541 | GACCACTACC AGCAGAACAC CCCCATCGGC GACGGCCCCG TGCTGCTGCC CGACAACCAC |
| 601 | TACCTGAGCA TCCAGTCCAA GCTGAGCAAA GACCCCAACG AGAAGCGCGA TCACATGGTC |
| 661 | CTGAAGGAGA GGGTGACCGC CGCCGGGATT ACACATGACA TGAACGAGCT GTACAAG |

hfYFP-S147P/V206K/L195M

The amino acid sequence of hfYFP-S147P/V206K/L195M is as follows:

```
(SEQ ID NO: 7)
    1 MVSKGEELFT GVVPILVELD GDVNGHKFSV RGEGEGDATN GKLTLKLIST TGKLPVPWPT
  61 LVTTLGYGLM VFARYPDHMK QHDFFKSAMP EGYVQERTIS FEDDGYYKTR AEVKFEGDIL
121 VNRIVLKGID FKEDGNILGH KLEYNFNPHN VYITADKQKN GIKANFKIRH NVEDGGVQLA
181 DHYQQNTPIG DGPVLMPDNH YLSYQSKLSK DPNEKRDHMV LKERVTAAGI THDMNELYK
```

hfYFP-G65S/Y203I/V206K

The amino acid sequence of hfYFP-G65S/Y203I/V206K is as follows:

| (SEQ ID NO: 8) | |
|---|---|
| 1 | MVSKGEELFT GVVPILVELD GDVNGHKFSV RGEGEGDATN GKLILKLIST IGKLPVPWPT |
| 61 | LVTTLSYGIM VFARYPDHMK QHDFFKSAMP EGYVQERTIS FEDDGYYKTR AEVKFEGDIL |
| 121 | VNRIVLKGID FKEDGNILGH KLEYNENSHN VYITADKQKN GIKANFKIRH NVEDGGVQLA |
| 181 | DHYQQNTPIG DGPVLLPDNH YLSIQSKISK DPNEKRDHMV LKERVTAAGI THDMNELYK |

hfYFP-G65S/Y203I

The amino acid sequence of hfYFP-G65S/Y203I is as follows:

| (SEQ ID NO: 9) | |
|---|---|
| 1 | MVSKGEELFT GVVPILVELD GDVNGHKFSV RGEGEGDATN GKLTLKLIST TGKLPVPWPT |
| 61 | LVTILSYGLM VFARYPDHMK QHDFFKSAMP EGYVQERTIS FEDDGYYKTR AEVKFEGDTL |
| 121 | VNRIVLKGID FKEDGNILGH KLEYNENSHN VYITADKQKN GIKANFKIRH NVEDGGVQLA |
| 181 | DHYQQNTPIG DGPVLLPDNH YLSIQSVLSK DPNEKRDHMV LKERVTAAGI THDMNELYK |

### LSSA12

The amino acid sequence of hfYFP-G65S/Y203I/Q204E/E222D/R223F, which we named LSSA12, is as follows:

| (SEQ ID NO: 10) | |
|---|---|
| 1 | MVSKGEELFT GVVPILVELD GDVNGHKFSV RGEGEGDATN GRETLKLIST TGKLPVPWPT |
| 61 | LVTILSYGLM VFARYPDHMK QHDFFKSAMP EGYVQERTIS FEDDGYYKTR AEVKFEGDTL |
| 121 | VNRIVLKGID FREDGNILGH KLEYNFNSHN VYITADKQKN GIKANFKIRA NVEDGGVQLA |
| 181 | DHYQQNTPIG DGPVLLPDNH YLSIESVISK DPNEKRDHMV LKDFVTAAGI THDMNELYK |

mT-Sapphire

| (SEQ ID NO: 11) | |
|---|---|
| 1 | MVSKGEELFT GVVPILVELD GDVNGHKFSV SGEGEGDATY GKLILKFICT TGKLPVPWPT |
| 61 | LVTTFSYGVM VFARYPDHMK QHDFFKSAMP EGYVQERTIF FKDDGNYKIR AEVKFEGDTL |
| 121 | VNRIELKGID FKEDGNILGH KLEYNFNSHN VYIMADKQKN GIKANFKIRH NIEDGGVQLA |
| 181 | DHYQQNTPIG DGPVLLPDNH YLSIQSKLSK DPNEKRDHMV LLEFVTAAGI TIGMDELYK |

mT-Sapphire-V68Q

| (SEQ ID NO: 12) | |
|---|---|
| 1 | MVSKGEELFT GVVPILVELD GDVNGHKFSV SGEGEGDATY GKLILKFICT TGKLPVPWPT |
| 61 | LVTIFSYGQM VFARYPDHMK QHDFFKSAMP EGYVQERTIF FKDDGNYKIR AEVKFEGDTL |
| 121 | VNRIELKGID FKEDGNILGH KLEYNENSHN VYIMADKQKN GIKANFKIRH NIEDGGVQLA |
| 181 | DHYQQNTPIG DGPVLLPDNH YLSIQSKLSK DPNEKRDHMV LLEFVTAAGI TLGMDELYK |

### Development of Cysteine-Free Fluorescent Proteins

The two highly conserved cysteine residues in avFPs are spatially separated and cannot form a disulfide bond under native conditions (FIG. 7A), but they enable an important misfolding pathway in avFPs through interchain disulfide bond formation as the nascent polypeptide is processed through the secretory pathway. Counter-intuitively, nearly all substitutions to C48 and C70 result in nonfluorescent protein, suggesting that cysteine substitution may destabilize the protein structure, and/or the folding process through a disulfide-independent mechanism. The best-tolerated substitutions appear to be C48S and C70V. Although introducing the “cycle-3” or “superfolder” mutations can partially rescue brightness, double mutants rarely retain their original fluorescence for reasons that are not known.

We produced C48S/C70V double mutants of eGFP, Clover (Lam et al., 2012), and mF1Y (an intermediate from mGreenLantern development) (FIG. 8), and compared them to the original proteins. We also included the human codon-optimized sfGFP-C48S/C70S variant, “moxGFP”, to accompany sfGFP. Whereas E. coli colonies expressing eGFP-C70V were practically nonfluorescent, the Clover-C70V colonies appeared minimally affected. To quantify the brightness of C48S/C70V double mutants in cells, we used an established co-expression strategy that generates an FP and mCherry in a roughly equimolar ratio, allowing normalization to the red fluorescence and thereby controlling for transfection-related variability. Consistent with our observations in E. coli, eGFP-C48S/C70V was mostly nonfluorescent in human cells, while moxGFP (sfGFP-C48S/C70S) was slightly dimmer than eGFP and half as bright as sfGFP. Interestingly, Clover-C48S/C70V was brighter than Clover in all three human cell lines tested, displaying 2.7-fold greater brightness than eGFP, compared to 2.2-fold for Clover relative to eGFP. mF1Y and its double mutant were both 3.5-fold brighter than eGFP (FIG. 7B).

To assess the impact of cysteine substitutions on protein stability, we experimentally determined FP melting temperatures (Tₘ) and found that the C48S/C70S mutations of moxGFP completely eliminated the thermodynamic stability benefit conferred by the superfolder mutations: moxGFP's Tₘ of 79.5° C. is approximately the same value as eGFP's and Clover's (FIG. 7C). Interestingly, Clover-C48S/C70V displayed a minor secondary melt peak at 89.6° C., higher than sfGFP's single peak of 86.4° C. Clover-C48S/C70V's primary Tₘ peak decreased by only 3° C. compared to the 7° C. drop seen between sfGFP and moxGFP. The secondary peak (FIG. 7C, arrow) was visible as a distinctive phase that was absent in the other FPs (FIG. 7D). The data suggest that a unique structural change occurs at high temperature in Clover-C48S/C70V that stabilizes the protein and/or shields the chromophore from quenching.

There were no obvious differences in typical spectroscopic characteristics that could satisfactorily explain the cellular brightness and Tₘ differences between the cysteine-replaced variants and their parents (FIG. 7E), indicating that folding and/or chromophore maturation processes might be hindered. Indeed, we found that chromophore maturation occurs 35% more slowly in moxGFP than in sfGFP, while Clover-C48S/C70V matures at half the rate of Clover (FIG. 7E). The data imply that delayed chromophore maturation in these mutants arises from thermodynamically unfavorable interactions during the protein folding process that ultimately yield a destabilized final structure.

Although it might appear that eliminating undesirable interchain disulfide bond formation by removing cysteines solved one folding problem and introduced another, the presence of a second melt peak in Clover-C48S/C70V raised the possibility that the avFP thermodynamic equilibrium could be shifted toward a more stable final conformation using structure-guided engineering, perhaps without even modifying intrinsic spectral properties.

To test that hypothesis, we applied the C48S/C70V mutations to various mGreenLantern mutants and re-introduced mutations that we had removed previously while optimizing for brightness, including those most closely attributed to the stability of “superfast GFP” variants: mutations K101E, N105Y, E124V, G232D, and D234N, as well as the “superfolder” A206V mutation. Some mutations, such as F223R, we reverted back to wild-type (FIG. 8), since the L221K mutation is enough to monomerize eGFP and we did not know what role F223 might play in protein stability.

Introducing C48S/C70V into F3C and related variants produced a series of FPs with Tₘ values ranging from 84-88° C., like sfGFP's (Tₘ=86.4° C.) (FIG. 9A), despite the absence of cysteine residues, which we have shown are detrimental to the thermodynamic stability of eGFP-C48/C70V and moxGFP (FIGS. 7C-7E). The foundational protein of our “hyperfolder” library was FOLD4, a C48S/C70V mutant that we constructed as a hybrid template containing the superfolder GFP (Pédelacq et al., 2006) and superfast GFP “P7” (Fisher and DeLisa, 2008) mutations in the Clover background (characterized by T65G/Q69A/T203H mutations relative to sfGFP) (FIG. 8), along with several mutations from Emerald that improve E. coli colony brightness (Table 1). Introducing the V68L mutation common to YFPs raised the Tₘ of a mutant coded “FOLD6” to 90.0° C., approximately the value of the second melt peak in Clover-C48S/C70V (FIGS. 7C-7D).

### Role of V68L in Tm and Maturation Rate

Our finding that V68L raised thermostability above the apparent local maximum of Tₘ=84-88° C. for the explored sequence space of the FOLD4 variants marked a turning point in our development of stability-enhanced FPs. The V68L mutation is widespread in avFPs because it improves the chromophore oxidation rate of YFPs (GFP-T203Y variants) through structural rearrangements including H-bonding of the central α-helix main chain to structural water molecules involved in the proton wire. Moreover, for our purposes here, V68L was present in the superfast GFP library template that influenced our development of this series of proteins, perhaps indicating a beneficial role in folding and/or structural stability.

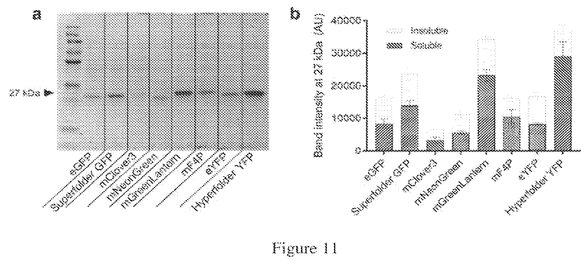

Consistent with a previous report suggesting a photostability improvement at the cost of maturation rate, V68L did indeed slow chromophore maturation when introduced into F4P to produce FOLD6, doubling the latency to completed chromophore cyclization compared to mF4P, to 25 min. That rate, however, was still faster than the maturation rate of eGFP, sfGFP, moxGFP, mClover3, and eYFP, among others (Table 2). The V68L mutation of FOLD6 also improved the rate of refolding to the half-maximal value relative to mGL, but with reduced slow-phase kinetics of its double-exponential curve (FIG. 10B). Performing the refolding experiment without reducing agent DTT completely prevented all the cysteine-containing FPs (eGFP, sfGFP, Clover, and mGL) from refolding. The refolding rates and curve fits were practically identical in the DTT(−) condition (FIG. 10), confirming that cysteine oxidation was responsible for the refolding deficit observed under these in vitro experimental conditions.

### The YFP Templates

Suspecting we had reached a Tₘ ceiling in the Clover-type GFP background (with His203), we generated YFPs carrying Tyr203. We blended features of Citrine, Venus, and YPet in a structure-guided manner, excluding any mutations that we deemed superfluous or application-specific, such as the various mutations in YPet that enhance FRET through dimer interface modifications (e.g., S208F and V224L were excluded). Introducing the F46L/A69M/H203Y/D234N mutations into FOLD6 produced the first of several YFPs with melting temperatures exceeding 90° C. (FIG. 9B) and cellular brightness values equal to or greater than eYFP's (FIG. 9C).

In addition to the FOLD6 (F4P-V68L) experimental data (Tₘ=90.0° C.) (Table 2), foxY. Citrine is a YFP with low acid sensitivity (pKₐ=5.7), improved refolding, and greatly reduced chloride sensitivity compared to eYFP and to Venus. Intriguingly, Citrine has been found to tolerate C48L/C70V substitutions, although the mutant was not extensively explored. Additionally, we recognized that T-Sapphire, a well-folded GFP with a large Stokes shift (LSS), features the Q69M and C70V mutations, the latter of which reportedly originated from a spontaneous PCR error. The presence of Q69M/C70V in the well-folded T-Sapphire and the possibility that these mutations would be compatible with Citrine instilled further confidence that our proposed YFP template would at least tolerate cysteine substitution.

We included the F46L mutation from Venus that purportedly improves chromophore maturation rate and refolding kinetics in a YFP-specific manner, perhaps due to the concerted effects of multiple “YFP-type” background mutations including T65G/V68L/T203Y, which we included as well. T203Y is the defining mutation that produces the bathochromic shift separating the GFP and YFP spectral classes.

Lastly, we re-introduced a mutation that we had tested previously in our early GFPs, D234N, which was first reported in a FRET-optimized bright YFP, YPet. The D234N mutation was later identified independently alongside G232D in one of the “superfast GFP” mutants, reinforcing our impression that the avFP C-terminus, which is usually unstructured in X-ray diffraction data, might play some role in protein stabilization. The mutations F46L/V68L/A69M/H203Y converted FOLD6 into foxY, which we then monomerized using L221K and F223R (to produce mfoxY), supposing that including both mutations would be advantageous in the more dimer-prone A206V background. As expected, the foxY proteins showed YFP-type excitation and emission maxima, but the quantum yield (QY) was 0.51, below the value of most avYFPs, including eYFP (Φ=0.60) (Table 2). Therefore, we next sought to improve the QY.

### Thr167 Reversion to Ile167 in YFPs Improved the Spectroscopic Properties

By sequence and structure alignment, we could find no avYFP containing Thr167, and, we suspected that Ile167 would promote favorable vdW interactions with the chromophore and with nearby F165 in the packed protein core. Re-introducing the wild-type Ile167 into mfoxY to produce mfoxYti (hfYFP) increased the QY by 20% to 0.61 and the EC to nearly 120,000 M⁻¹ cm⁻¹ without changing the pKₐ (5.5) (Table 2) or the GdnHCI resistance (FIG. 9D). Preliminary data suggest that the reversion doubled the photostability during laser-scanning confocal microscopy. Additionally, this reversion to Ile167 accelerated chromophore maturation by another 16% to 21 min, which is faster than eGFP and eYFP's (t_{1/2}=28 and >37 min, respectively). It also increased the Tₘ from 92.8 to 94.2° C. (Table 2). All YFPs from this series displayed cellular brightness values 2- to 3-fold greater than eGFP's. We named the mfoxY-T167I variant hyperfolder YFP (hfYFP) and fully characterized it (FIG. 1).

hfYFP is mGL-F46L/C48S/V68L/A69M/C70V/K101E/T105Y/K149N/T167I/H203Y/K206V/D234N.

### Mechanisms for Maturation Enhancement by the I167T Mutation

Our mF4P and FOLD6 point mutants allow us to define structure-function relationships resulting from the I167T mutation and, more generally, suggest a potential tradeoff between solvent accessibility that accelerates the chromophore cyclization rate at some cost to stability of the final structure.

The I167T mutation has been known to diminish the 405 nm absorbance (A-band) that arises from the protonated chromophore species. Most of our mutants carry the I167T mutation (Table 1) based on our hypothesis that it contributes to mGreenLantern's rapid chromophore maturation. Indeed, most of our GFP mutants show rapid chromophore maturation. mF4Y-SR exhibits the fastest maturation time that we've recorded, requiring only 6 min to reach half-maximal fluorescence intensity after ambient air introduction (Table 2).

Structurally, the I167T mutation in FOLD6 enables a 2.9 Å H-bond between T167 O_{γ1} and H169 N_{ε2}, shifting the imidazole side chain 1.0 Å upward toward T167 and increasing the channel diameter by 1 Å. A new H₂O molecule is found H-bonded to the T167 side chain in the polar core of the protein adjacent to H181, which is now tilted 30° directly toward T167, but only in the FOLD6 structure (the others contain Ile167). Distances between the nearest H₂O molecules (≤2.0 Å) in our 1.2 Å resolution structure indicate more heterogeneous occupancy than the rigidly constrained water molecules lying closer to the central α-helix. Altogether, this new configuration eliminates the hydrophobic barrier of isoleucine's side-chain and enables an uninterrupted H-bond circuit through H169 all the way into the protein's polar core (FIG. 17D).

Although surface contours do not explicitly map an open channel through T167-H169 in the mature FOLD6 protein (or in any avFP, to our knowledge), it is highly probable that the channel is sufficiently polar to conduct water molecules at least during the final steps of folding and barrel closure while chromophore cyclization proceeds as a parallel process. Such hydrated channels are of demonstrable importance to the chromophore maturation of TurboGFP, an FP derived from Pontellina plumata. Water exchange may also occur in the mature protein, as suggested by molecular dynamics (MD) simulations of the avFP β-barrel, and may influence its function. MD simulations probing organic solvent exchange at the β-barrel interface would be valuable to understanding the susceptibility or resilience of FPs to various tissue clearing reagents or fixatives, such as those we tested in FIG. 3, and might lead to further optimized variants.

We probed structure-function implications of the I167T mutation experimentally. Introducing H169L to disrupt the described T167-H169 H-bond (FIG. 17D) increased the pKₐ from 5.9 to 6.7 and decreased cellular brightness by 50%, a substantial drop that can be primarily attributed to the pKₐ shift. From a structural standpoint, the aliphatic leucine side-chain that replaces histidine increases the hydrophobic character of the hydrated tunnel and opposes the hydrophilic advantage (including the H-bond) conferred by the Thr167 side-chain. Our experimental data support this assertion, as reverting T167 to wild-type I167 in mF4P (producing mF4Pti) doubled the maturation latency and halved the cellular brightness (Table 2). Although the QY improvement from 0.75 to 0.77 in mF4Pti relative to mF4P is within the experimental error range, it may be significant, and it is conceivable that Ile167 may stabilize the chromophore and F165 through vdW forces that rigidify this region and decrease nonradiative decay of the excited state. If so, the stabilizing benefit of Ile167 may come at the cost of a maturation advantage afforded by smaller residues such as Thr.

If vdW contacts between the chromophore and F145 are useful for decreasing nonradiative quenching of the excited state, which is likely, then that might also partly explain the decreased QY of hfYFP relative to the GFPs, since the F165 side chain is considerably shifted away from the chromophore in hfYFP and mhYFP, and even more so in FOLD6, yet the chromophore remains in the same position. Decreased friction from longer and weaker vdW associations with F165 might increase the probability of nonradiative de-excitation through chromophore twisting motion in hfYFP. Chromophore planarity might also play an important role.

Roger Tsien's group suggested that the I167T mutation stabilizes the B-state, which at the time was suspected but not definitively demonstrated to represent the deprotonated chromophore (Heim et al., 1994). Later work has suggested that the tip of Ile167 Cδ1 makes an unfavorable contact with the chromophore phenolate that stabilizes the A-state and that this interaction is relieved by Thr167, thereby enhancing the B-state. The authors observed the same T167-H169 H-bond that we report here in several I167T-containing structures. In addition to these observations, we propose based on our the crystallographic and functional data, that I167T shifts the chromophore equilibrium to the predominantly deprotonated state by stabilizing H169 through the T167-H169 H-bond and opening the polar channel to exert long-range effects into the protein's hydrophilic core (FIG. 17), which also accelerates chromophore maturation. Presumably, E222 remains protonated (and the chromophore deprotonated) because the FOLD6 H-bond network minimizes environmental changes to the proton wire that would alter the protonation status of either residue, with interactions resulting from the I167T mutation further conferring a low pKₐ (FIG. 17D). We interpret this function of I167T through the pKₐ-reducing effect that decreases the A-band—shifting the pH curve—while the hydrated channel from outside the protein into the chromophore environment accelerates chromophore maturation during folding.

This explanation may account for the marginal EC drop in FOLD6-H169L relative to FOLD6 (118 to 108 mM⁻¹ cm⁻¹), as well as the lack of benefit to our YFPs, all of which contain the Q69M mutation (relative to eGFP, eYFP, and Venus) that strengthens the chromophore's hydrophobic packing as discussed and leaves no space above the chromophore for water molecules to reside (FIG. 18B), in contrast to the open cavity in FOLD6 (and in the Clover structure) that originates from Clover's Q69A mutation and influences the proton wire of both (FIG. 18B). This key structural difference between the GFPs with A69 and YFPs with M69 may explain why reverting Thr167 in mfoxY to Ile167 in mfoxYti (hyperfolder YFP)—like the T167I reversion that produced mF4Pti—increased the QY and EC greatly without appreciably changing the pKₐ (Table 2): the hfYFP chromophore environment is already tightly packed and more hydrophobic than those of H203-A69 GFPs such as Clover, mF4P, and mGL.

### Additional Observations Related to Chromophore Maturation

Our spectroscopically characterized library data allow us to generate new conclusions about existing structures, and we mention several examples here. One is that mClover3's slow maturation compared to Clover's (t_{1/2}=37 min vs. 15 min) is primarily due to its G160C mutation, and perhaps partly due to V206K reversion (compare the maturation rate of mF2BK^{DMD} to mGreenLantern's in Table 2; the difference is the V206K mutation), but is not due to N149Y, which improved maturation in at least two of our GFPs (Table 2) but nearly quadrupled the maturation time of mfoxY relative to mfoxYY (25 min vs. 99 min, respectively. Additionally, we can conclude from our data that the V68L mutation that differentiates mF4P from FOLD6 (besides the phenotypically neutral L221K monomerizing mutation) is completely responsible for delaying FOLD6 maturation 2-fold relative to mF4P (Table 2).

We speculate that the F46L mutation of Venus, which is reported to improve the maturation rate of YFPs, may serve to counteract the maturation-delaying effect of V68L, perhaps alongside the F64L mutation that serves that role, through positional adjustment of the L68 main-chain and its proximity to wat₂ (FIG. 17A) and through a chain reaction of alkyl side-chain adjustments in the hydrophobic region of the protein, part of which is depicted in FIG. 17C, clockwise from E222 to N121 (the L68 main-chain is pictured, but the side-chain was omitted for clarity in the context in which the figure is discussed).

Hydrophobic Packing in the hfYFP Chromophore Environment

Another protein that has been engineered for tissue clearing applications is muGFP, or sfGFP-Q69L/N164Y/F223D (Scott et al., 2018). Using their muGFP crystal structure (PDB: 5JZL), the authors concluded that elimination of a water molecule in the chromophore environment and greater hydrophobic packing, among other reasons, contributed to the improved reported stability of muGFP relative to eGFP.

Likewise, we have concluded in our study that the Q69M mutation in hfYFP (relative to eGFP), would improve hydrophobic packing within the protein not just by eliminating water molecules, but via the potentially stabilizing effects of sulfur-aromatic interactions of Met69 with Phe84, Tyr203, and the chromophore itself (FIG. 18). Methionine-aromatic interactions have been described and quantified, and in many cases these interactions play an important and unique role in protein structure stabilization.

Further efforts to develop FPs for tissue clearing and super-resolution applications may benefit from structural examination of the chromophore packing environment and identification of suitable templates, such as hfYFP and LSSmGFP, that can tolerate extensive hydrophobic interactions while maintaining the specific H-bond networks that are critical for producing the desired fluorescence spectra (FIGS. 17A-17B).

### a Third Water Molecule in the Chromophore Proton Wire

The FOLD6 structure reveals an unusual water molecule, wat₃, that is also found in roClover0.1 (structure solved to 1.3 Å resolution), but only in the B-chain. It does not appear in Clover, perhaps due to limitations of the structure's solved resolution (2.4 Å). The presence of wat₃ in these examples is interesting because, to our knowledge, the only other structure that renders wat₃ is the 1.7 Å resolution off-state of Dreiklang (Citrine-V61L/F64I/Y145H/N146D) where it is referred to as “wat_{c}”. It does not appear in the on-state (2.0 Å resolution) or the equilibrium state structures. Whether or not wat₃ plays a role in the chromophore H-bond network in Dreiklang is not entirely clear, but in the FOLD6 structure, which shows a single E222 conformation, the proximity of wat₃ to the polar side-chains of S205 and E222 appears to form two H-bonds to stabilize the proton wire and, presumably, maintain a protonated E222 side-chain and deprotonated chromophore (FIG. 17B).

Effort to Develop a Photoswitchable hfYFP

We briefly attempted to develop a “hyperfolded” cysteine-free Dreiklang variant by merging the FOLD6/hfYFP proton wires in a structure-guided manner, recognizing that core features of Citrine in hfYFP, such as the Q69M mutation, could be compatible with Dreiklang's switching behavior that has been structurally described. To summarize this effort, applying the core Dreiklang mutations V61L/F64I/Y145H/N146D to hfYFP did not produce a photoswitchable protein, but reverting three mutations at the avFP dimer interface indeed yielded a photoswitchable mutant with fluorescence excitation decoupled from switching, hfYFP-V61L/F64I/Y145H/N146D/V206A/K221L/R223F, albeit with less efficient switching compared to Dreiklang. We did not pursue this line of inquiry further, but we surmise that the presence of a weak dimer interface may be necessary for the unique switching behavior of Dreiklang.

### Slow Denaturation in NaOH

Regarding unusual protein stability, we are aware of one example of comparably slow denaturation in the 1 M NaOH solution (FIG. 14) that is used to obtain extinction coefficient (EC) values: the eqFP611 template from which mRuby was engineered. Of course, there are considerable differences between anemone- and jelly-derived FPs, but clearly there is a functional similarity that suggests a common structural element. Our data suggest that rigidification of β-strand 7, perhaps through the S147P mutation (mF4P, FOLD6, and mhYFP all feature it), contributes to the extended NaOH tolerance. Upon aligning the hfYFP and mRuby structure (PDB: 3U0M), there is a proline residue at the equivalent mRuby position, within a tighter β-strand 7 turn that may be expected to decrease the strand's conformational heterogeneity. Further studies of the hfYFP structure, including molecular dynamics simulations, could lend deeper insight into these peculiarities and further advance the engineering of new FPs for biotechnological applications.

### EXAMPLES

The invention now being generally described, it will be more readily understood by reference to the following examples that are included merely for purposes of illustration of certain aspects and embodiments of the present invention, and are not intended to limit the invention.

### Example 1. Methods

### Plasmid Construction and Cloning

All FPs in this study (besides mScarlet-I) were designed to maintain the seven avFP-type N-terminal amino acids, MVSKGEE, encoded by nucleotide sequence ATGGTGAGCAAGGGCGAGGAG. Likewise, the C-terminal avFP amino acids, GMDELYK (nucleotides CGGCATGGACGAGCTGTACAAG), were maintained in all FPs except those carrying the G232D and G232D/D234N mutations (resulting in GACATGGACGAGCTGTACAAG and GACATGAACGAGCTGTACAAG, respectively). PCR primers 21 nucleotides in length were designed using those sequences in the appropriate orientation to amplify the FP gene, or the entire host plasmid as a linearized empty vector. This primer design strategy maintains proper stop codon placement. FP gene amplicons and linearized empty vectors with the compatible termini were amplified, purified, and quality-controlled so that any combination of FP gene and vector could be cloned on-demand from the same insert/vector stocks using restriction site-independent isothermal “Gibson” assembly of the overlapping DNA fragments. This approach works well for simple plasmids like pBAD and pcDNA3.1 but should not be used for plasmids with repetitive elements or complex secondary structure, such as viral vectors. Consequently, cloning into the adeno-associated virus (AAV) expression vector pAAV-CAG-FLEX was performed using standard T4 ligation between the BamHI/EcoRI sites.

Fusion constructs depicted in FIG. 5F were cloned into a pET28a vector modified to remove the N-terminal thrombin cleavage site while preserving the N-terminal hexahistidine (His₆) tag. The full pET28a-hfYFP plasmid was amplified to linearize it between the 3′ end of hfYFP and vector backbone. The oligonuclotides provided long overhangs coding for the first half of a linker at the 3′ end of the hfYFP sequence, while the fusion protein genes (mScarlet-I, Bacillus circulans xylanase, or streptavidin) were amplified using oligos to complete the linker (GSAGSAAGSGEFENLYFQGH) at the 5′ end of the gene and hybridize with the pET28a backbone on the 3′ end. The complete circular plasmid was generated from these two fragments using Gibson Assembly.

### Plasmid Sources and Cloning Material

All plasmids used in this study were generated from plasmids or gene synthesis products described in (Campbell et al., 2020), except for those listed in this section. pEGFP-N1-moxGFP was obtained from Addgene (#68070). pCytERM_mScarlet-i_N1 was obtained from Addgene (#85066). The streptavidin core domain sequence, constituting amino acids 13-140 of the native protein and responsible for its activity, was synthesized without further codon optimization. Bacillus circulans xylanase (synonymous with Niallia circulans endo-1,4-beta-xylanase) was synthesized from UniProtKB/Swiss-Prot: P09850.1 amino acid sequence using a bacterial codon set (Eurofins Genomics).

### Site-Directed Mutagenesis

Site-directed mutagenesis was performed using the QuikChange (Stratagene) method with Pfu polymerase (Agilent), or using the QuikChange Lightning Multi Site-Directed Mutagenesis Kit (Agilent) as described (Campbell et al., 2020). Non-phosphorylated mutagenic primers were designed to introduce the most abundant human codon for the target amino acid. Degenerate codon selection for multi-site mutagenesis were performed manually or facilitated by the SwiftLib program (Jacobs et al., 2015). Whole genes were not codon-optimized: the nucleotide background ultimately originates from Clover (Lam et al., 2012), which is based on eGFP. For the Clover nucleotide sequence, refer to Addgene #215680.

### Construction and Screening of Structurally Targeted Libraries

To generate small structurally targeted libraries to generate 405 nm excitable FPs, specific residues were selected for mutagenesis based on the hfYFP crystal structure as described in FIG. 5B. When modifying multiple adjacent codons (e.g., to mutate β-strands 10 and 11), primers were designed to amplify large sections of the FP gene with overhangs at junctions between the segments containing the standard or degenerate codons targeting those sites, followed at the 3′ end by a homology arm for the adjacent gene fragments amplified in separate reactions. The gene fragments containing these degenerate codons at the junctions were stitched together by overlap-extension PCR (Heckman and Pease, 2007), gel purified, and cloned by isothermal assembly (Gibson, 2011) into a PCR-amplified linear pBAD vector for transformation and expression in E. coli.

Mutated FP genes were transformed into TOP10 competent cells and grown at 37° C. on LB agar plates supplemented with carbenicillin (100 μg/μL) and 0.02% arabinose. The next day, colonies were screened by eye using alternating 405 nm and 470 nm LED strip illumination while fluorescence was observed through amber long-pass filter goggles (Invitrogen #S37103 or ThorLabs #LG10). Colonies that glowed brightest under 405 nm excitation while showing minimal fluorescence under 470 nm illumination were picked into sterile 96-well deep-well blocks containing 1 mL LB medium supplemented with ampicillin (100 μg/μL) and 0.2% arabinose. The culture blocks were sealed with a breathable adhesive (EasyApp Microporous Film, USA Scientific #2977-6202) to permit air and gas exchange while minimizing evaporation, and cultures were grown at 37° C. with 275 rpm shaking for 16-18 hr. The following day, 100 μL overnight culture was pipetted into black clear-bottom 96-well optical plates (Corning) for first-pass excitation scans, and the ratio of 405 nm to 488 nm excitation was scored. Soluble protein from cultures with the greatest 405/488 nm ratio scores were extracted using B-PER II reagent (Thermo Scientific), re-scanned for confirmation, and the same lysate was then used for kinetic unfolding screens.

### Error-Prone Libraries

Error-prone libraries were generated using the staggered extension process (StEP) (Zhao and Zha, 2006) with some modifications. Plasmid DNA from 12 mutants from the LSSA12 library was pooled and diluted 50% with hfYFP-V206K/G65S/Y203I plasmid. To increase error rate, the Taq polymerase-based PCR reaction containing 33 ng total template DNA and 30 pmol of forward and reverse primer (flanking the complete FP gene), was supplemented with 0.5 mM MnCl₂. The thermal cycling program consisted of denaturation at 94° C. for 30 s and combined annealing/extension steps at 55° C. for 5 s, repeated 100 times before the reaction was cooled to 4-10° C. The PCR product was digested for 5 min using DpnI to eliminate residual parental plasmid, and the reaction was PCR purified. Upon analysis by gel electrophoresis, a single 720 bp band was observed. To generate additional product for cloning and storage, the StEP library was amplified using Phusion HS II polymerase (Thermo Fisher) with the same flanking primers, and the reaction product was cloned without further purification by isothermal assembly into PCR-amplified linear pBAD vector and expressed and screened as described.

### Protein Purification

Fluorescent proteins were purified as described (Campbell et al., 2020) using Ni-NTA chromatography, without lysozyme or DNAse for the large scale 500 mL preparations. Samples were dialyzed into TN buffer (50 mM Tris-HCl, 150 mM NaCl, pH 7.5), concentrated when necessary, flash frozen in TNG buffer (TN plus 10% glyccrol) using liquid N₂, and stored at −80° C. Protein for X-ray crystallography was prepared as described (Campbell et al., 2018) using pET28a-hfYFP, pET28a-mhYFP, and pET28a-FOLD6 vectors and purified by Ni-NTA chromatography. After overnight dialysis in TN buffer, gel filtration chromatography was performed using a HiPrep 16/60 Sephacryl S-200 HR column at a flow rate of 0.5 mL/min and fractions were collected while monitoring absorbance at 280 nm. The eluted FPs were filtered through an 0.22 μm membrane and concentrated to 20-40 mg/mL using Amicon centrifugal filter units (EMD-Millipore).

### Fluorescence-Assisted Purification of Inclusion Body Proteins Under Denaturing Conditions

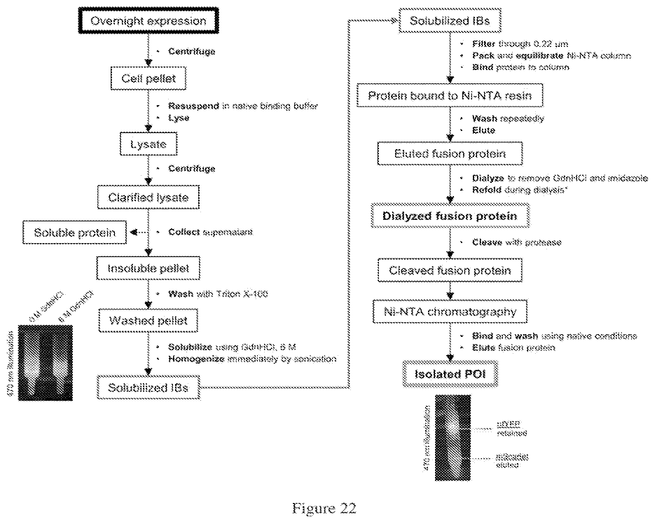

Fusions of hfYFP and mScarlet-I, Bacillus circulans xylanase (Bcx), or streptavidin (SAV) (see flowchart in FIG. 22) were transformed into E. coli TOP10 cells and expressed overnight in 50 mL LB medium supplemented with 100 μg/μL ampicillin and 0.2% arabinose. All fusions were grown at 37° C. for 18 hr, pelleted in 50 mL conical tubes, and frozen at −80° C.

Strips of ultraviolet (405 nm) and blue (470 nm) LEDs with a wide viewing angle were affixed to a shelf approximately two feet above the work surface and connected to toggle switches. All steps of the following purification process could be visualized under 405 or 470 nm LED illumination as desired for LSSmGFP or hfYFP, respectively, using orange filter goggles (Invitrogen).

The frozen pellets from 50 mL culture were thawed at RT and lysed in B-PER II reagent (Thermo Scientific) supplemented with DNAse I. After 5-10 min incubation at RT, lysate was sonicated on ice until homogeneous, transferred into several 1.5 mL Eppendorf tubes, and centrifuged at 20,000×g for 10 min at 4° C. in a benchtop microcentrifuge. The soluble fraction was collected. The insoluble pellet was washed with PBS plus 1% Triton X-100, briefly sonicated, centrifuged, and the supernatant containing residual soluble protein was discarded. This inclusion body pellet was washed twice more as described using PBS without the Triton X-100. The IBs were resuspended in Denaturing Purification Buffer (20 mM phosphate, 300 mM NaCl, 6 M GdnHCl, pH 7.4) containing 10 mM imidazole, rapidly and without trituration, and were immediately sonicated on ice until completely solubilized. Under these conditions, the IB pellets typically dissolved in ~10 s, producing a brightly fluorescent and homogeneous solution.

The fusion proteins were purified from solubilized IB solutions using HisPur Ni-NTA resin (Thermo Scientific, #88223). All purification steps were performed using the mentioned Denaturing Purification Buffer containing imidazole at a final concentration appropriate for equilibration, washing, or elution steps (10, 25, or 250 mM imidazole, respectively). Protein elution was monitored under LED illumination, and the fluorescent eluate was collected. The eluate was loaded into 10K MWCO dialysis cassettes (Thermo Scientific) and dialyzed in 50 mM Tris-HCl, pH 8.0, with gentle stirring at 4° C. overnight.

The concentration of the dialyzed sample was measured using its computed molecular extinction coefficient and absorbance at 280 nm. Next, hexahistidine (His)-tagged AcTEV protease (Invitrogen, #12575023) was added for cleavage according to manufacturer instructions. IMAC-incompatible chemicals such as DTT and EDTA were omitted. Cleavage proceeded at 4° C. for at least 24 hr.

The cleaved Protein of Interest was then isolated by Ni-NTA chromatography under native conditions in the mentioned Purification Buffer without GdnHCl. His₆-TEV protease and His₆-hfYFP or His₆-LSSmGFP are adsorbed onto the Ni-NTA resin while the Protein of Interest remains in solution. After binding, the column is unplugged and the flow-through containing the purified Protein of Interest is isolated. See FIG. 22 for a flowchart. Enzymatic activity of SAV was quantified as described using fluorescence quenching of biotin-4-fluorescein (B4F) (CAS #: 1032732-74-3) (Kada et al., 1999).

Note: mScarlet-1 and SAV refolded during dialysis under the described conditions as part of the hfYFP fusion construct, but Bcx did not. Refolding of denatured proteins is a complex topic that is abundantly addressed elsewhere. Conditions to maximize refolding efficiency and catalytic activity of each protein of interest must be optimized. It is necessary to consider whether the refolding buffer is compatible with the specific protease that is intended to cleave the construct (in this case, TEV protease), also whether the refolding buffer is compatible with immobilized metal affinity chromatography (IMAC). Otherwise, dialysis steps should be included.

### Spectroscopy

Unless otherwise stated, all optical assays were performed using a BioTek Synergy H1 microplate reader. Samples were dispensed into black clear-bottom 96-well assay plates (Corning Costar) or into 1 cm path length micro-volume quartz cuvettes (Hellma Suprasil) placed horizontally in a BioTek Take3 microplate. Stability assays occurred in TNG buffer (50 mM Tris-HCl, 150 mM NaCl, 10% glycerol, pH 7.5) and used detection settings λₑₓ/λₑₘ=495/525 nm (or 405/525 nm for the LSS FPs), unless otherwise specified.

Absorbance measurements were collected in quartz cuvettes from 250-650 nm in 1 nm steps. Extinction coefficients (ECs) were determined using alkali denaturation (Cranfill et al., 2016). For slow denaturing Hyperfolder FPs that did not immediately show a single clean peak at ~447 nm from the initial 430-460 absorbance scan that was performed immediately after 1:1 dilution of FP into 2 M NaOH, a 10-30 min time-course was conducted using λ_{abs}=447 nm and 505 nm. To prevent artificial EC inflation, the maximum 447 nm absorbance value from the time-course (see FIG. 14) was chosen for calculation, thereby yielding the lowest possible EC value relative to other points in the data set, since absorbance and concentration are inversely proportional when ε is constant (44,000 M⁻¹ cm⁻¹ for the denatured GFP-type chromophore). Performed this way, the EC values for the slow-denaturing FPs largely fell within the typical EC range for bright GFPs and YFPs (Cranfill et al., 2016). This strategy, of course, applies to “fast-denaturing” avFPs just the same, since the first data point at ~447 nm will be the maximum value when the initial 430-460 nm absorbance scan shows that the FP has denatured.

Quantum yield (QY) values were determined as described (Campbell et al., 2018), using a PTI Quantamaster and the same quartz cuvettes from the absorbance measurements. Experimental samples were run in quadruplicate with the following cross-referenced controls for GFPs/YFPs: fluorescein in 0.1 M NaOH (φ=0.925); and Clover (φ=0.76) (Lam et al., 2012) and eYFP (φ=0.61) (Kremers et al., 2006) in PBS, pH 7.4. For the LSS-GFPs, the following standards were used: quinine sulfate in 0.1 M H₂SO₄ (φ=0.53) (Adams et al., 1977); and EBFP2 (φ=0.56) (Ai et al., 2007) and mT-Sapphire (φ=0.60) (Cranfill et al., 2016) in PBS, pH 7.4.

### Stability Assays

### Thermal Denaturation and Isothermal Melting

Fluorescent protein was adjusted to 1 μM final concentration in TNG buffer and 50 μL was dispensed into replicate wells of a clear 96-well qPCR plate. The plate was sealed with optical adhesive and heated in a Bio-Rad C1000 Touch thermal cycler equipped with a CFX96 Real-Time System and FAM filter. Temperature was brought to 25° C. and measured three times in 30 s intervals to obtain the baseline fluorescence value before heating at a rate of 0.3° C./min to a final temperature of 100° C. Data sets containing the negative first derivative of the change in fluorescence (−d(RFU)/dT) was exported from the Bio-Rad CFX96 software, and then background subtracted and normalized to the average of the baseline fluorescence value at 25° C. The melting temperature (Tₘ) is the x value when the normalized intensity y value=1. Melting temperatures for the LSS FPs were determined using the thermofluor assay as described (Huynh and Partch, 2015) using SYPRO Orange dye and the ROX filter set with the same equipment.

For the isothermal melting experiment, samples were prepared as described and the thermal cycler was programmed using the gradient setting to heat individual plate rows to 66.6, 69.8, 74.4, 80.1, 84.9, and 87.7° C. using the maximum ramp rate. The temperatures were maintained for 5 hr while fluorescence measurements were acquired every 1 min using the FAM filter set. Data were background subtracted and plotted relative to the first data point of the series.

### Kinetic Unfolding

Purified FP stocks were diluted 10-fold into TNG buffer, pH 7.5, with and without 7 M guanidinium hydrochloride (GdnHCl) (Fisher Scientific, #BP178-500), for final concentrations of 0.1 μM FP and 6.3 M GdnHCl (or 0 M GdnHCl for the native control samples). The plate was immediately sealed with optical adhesive (Bio-Rad), and the first fluorescent measurement (λₑₓ/λₑₘ=495/525 nm, or 405/525 nm for the LSS FPs) was recorded within ~15 s of dilution. Short-term and long-term unfolding curves were generated using 10 s or 1 min sampling interval for total experiment durations of 1 hr or 12 hr, respectively. The fluorescence ratio of the unfolding protein to the same FP's native control wells was plotted for each FP (“fraction folded”). Data points were fit to single- or double-exponential equations where indicated. Preliminary screening of mutant libraries for generating the large Stokes shift constructs was performed using clarified lysate and then confirmed using purified protein for final data sets. For initial screening the LSS FPs, clarified lysate was used, and purified protein was used for confirmation.

### Equilibrium Unfolding

Equilibrium unfolding experiments were conducted based on a reported method (Fisher and DeLisa, 2008). FPs were diluted to 0.1 μM final concentration in solutions of GdnHCl or GdnSCN in TNG buffer, pH 7.5, except that no dithiothreitol (DTT) was omitted in this experiment. Plates were sealed with optical adhesive and stored in the dark at RT for 24 hr before collecting measurements using endpoint scans of λₑₓ/λₑₘ=495/525 nm. Data were plotted relative to the fluorescence intensity value of the individual FP at 0 M Gdn (TNG buffer only).

H₂O₂ and Chloride Sensitivity Assays

Stocks of 1 μM FP were diluted 10-fold into solutions of H₂O₂(Fisher Scientific, #H325-100) in PBS, pH 7.4, for final concentrations of 0.1 μM protein and 0-27% v/v H₂O₂ (16 solutions). Fluorescence was measured after exactly 15 min incubation at RT, using endpoint scans at λₑₓ/λₑₘ=495/525 nm, or 405/525 mu for the LSS FPs. Timing is critical for achieving reproducible results in this experiment: H₂O₂ was used in great excess, even at the lowest concentrations, resulting in very rapid denaturation of all FPs within 30-60 min.

To measure chloride sensitivity, the same dilution procedure was used. Samples were then incubated in solutions of HEPES-NaOH, pH 7.5, with chloride concentrations ranging from 0-500 mM (16 solutions) for 24 hr in the dark at RT before measuring using endpoint scans at λₑₓ/λₑₘ=495/525 nm.

### Refolding

Purified protein in TNG buffer (50 mM Tris-HCl, 150 mM NaCl, 10% glycerol, pH 7.5) was diluted into a solution of 7 M GdnHCl prepared in TNG buffer and supplemented with dithiothreitol (DTT), for final concentrations of 1 μM protein, 6.8 M GdnHCl, and 1 mM DTT. The samples in this solution were fully denatured by heating at 98° C. for 10 min, cooled to RT, and briefly checked under a 470 nm handheld LED to confirm loss of fluorescence. Native samples in the same buffer without GdnHCI were prepared in parallel.

The BioTek Synergy H1 microplate reader was configured for rapid fluorescence endpoint scans at λₑₓ/λₑₘ=495/525 nm for 1 hr at 5 s intervals. 20 μL of native and denatured protein at 1 μM concentration was dispensed into empty wells of a black 96-well optical plate. Refolding was initiated by ejecting 200 μL TNG buffer supplemented with 1 mM DTT directly into the wells without further mixing (protein Cf=0.1 μM), and the kinetic scan was initiated within 10 s. The data were plotted as the value of the refolding sample divided by the native sample containing 0 M GdnHCl for each time point, thereby giving the fraction folded.

### Mammalian Cell Culture and Imaging

Low passage HeLa (ATCC CCL-2), BE(2)-M17 (ATCC CRL-2267) were routinely cultured in OptiMEM (Gibco) supplemented with 5% fetal bovine serum (VWR) and penicillin-streptomycin (Gibco). For localization experiments and OSER assay, HeLa cells were passaged into 35 mm culture plates containing a 22 mm glass bottom (MatTek) and grown for at least 24 hr before transfection using Turbofect (Thermo Fisher) and 1 μg plasmid. hfYFP folds very quickly, and some organelles naturally require the unfolded polypeptide to pass through a translocon for successful import before folding and expression in the target structure (Fisher and DeLisa, 2008; Kashiwagi et al., 2019). Therefore, new users of hfYFP/mhYFP—or any fast-folding FP—may wish to test several DNA concentrations beginning in the lower range for their specific transfection system to minimize cytosolic spillover, particularly for endoplasmic reticulum and mitochondrial targeting.

Live cells were imaged 12-18 hr after transfection using a Zeiss LSM 880 laser-scanning confocal microscope equipped with computer-controlled Zeiss Enhanced Navigation (ZEN) software; an argon-ion laser for with MBS 488 and MBS 458/514 beam splitters for GFP and YFs, respectively; Plan-Apochromat 10×/0.45 WD=2.0 M27 and Plan-Apochromat 40×/1.3 Oil DIC UVVIS-IR objectives; high-sensitivity GaAsP photodetector; configuration for bidirectional scanning and 12-bit image acquisition. FP fusion localization images were acquired at 2048×2048 px resolution with 4 times averaging and represent a single Z-section at 1 Airy unit. OSER assay images were tile scans (4×4) acquired at 1024×1024 px resolution with 4 times averaging using the 40× oil immersion objective. OSER images were stitched using ZEN software and analyzed using the established scoring criteria (Costantini et al., 2012).

Cross-excitation assays were performed by culturing and transfecting HeLa cells on 35 mm MatTek plates as described with individual plasmids (0.5 μg DNA each) encoding LifeAct-eGFP and H2B-mT-Sapphire, or LifeAct-mGreenLantern and H2B-LSSmGFP. The next day, cells were imaged on a Keyence BZ-X700 All-in-One Fluorescence Microscope equipped with a S PlanFluor ELWD ADM 40×C 0.60/3.60-2.80 mm Ph2 air objective, 470 nm ex 525 nm em GFP filter cube (Keyence), custom 405 nm ex 525 nm em Keyence BZX Cube (Chroma Technology Corp), and computer-controlled motorized stage. Images of 1920×1440 px resolution were acquired in 8-bit TIFF format under identical imaging settings for each of the LifeAct and H2B fusion proteins. Images were prepared using ImageJ.

### Fluorescence Retention, Fixatives

A fresh ampule of 32% (w/v) paraformaldehyde (PFA), methanol-free, was diluted to 4% (v/v) in PBS, pH 7.4. The remaining 32% (w/v) PFA liquid was then used to prepare a solution of 2% (v/v) PFA and 2% (v/v) glutaraldehyde (Glut) in PBS, pH 7.4, from a 25% (w/v) stock bottle that was stored sealed under refrigeration. A solution of 3% glyoxal in 20% absolute ethanol, pH 4.0, was prepared according to the recipe (Richter et al., 2018) from a 40% glyoxal stock bottle (Sigma, #128465). PBS++ was prepared by supplementing a 10×PBS, pH 7.4 (Gibco) stock bottle with MgCl₂ and CaCl₃ solutions before dilution to 1× working concentration and 0.1 mM final concentration of each salt.

HEK293T cells were cultured in Matrigel-coated black 96-well tissue plates and transfected with cytosolic expression plasmids using 0.1 1.4 DNA per well and Turbofect transfection reagent. Media was changed the following morning. After 48 hr total expression time, cells were washed twice using phenol red-free medium and imaged at 10× magnification on the mentioned widefield Keyence BZ-X700 plate reader microscope using a PlanFluor DL 10× 0.30/15.20 mm Ph1 air objective, GFP filter cube for GFPs and YFPs, or the mentioned 405/525 nm filter cube for LSS FPs. Manually focused images of 960×720 px (2×2 binning) resolution were acquired in 8-bit TIFF format. Imaging settings were specific to each FP to provide optimal exposure without oversaturation. Acquisition settings were recorded, and the same settings were used for pre-fixation and post-fixation imaging per FP.

After imaging the live cells (pre-fixation image), samples were incubated with room temperature (RT) fixatives for 15 min at RT. Afterward, the cells were gently washed 3 times for 5 min each using PBS++. The washed cells were imaged using the settings loaded from the appropriate live cell fluorescence image for each well. Pre- and post-fixation images were registered using ImageJ. The live cell image was thresholded, cell regions of interest (ROIs) were applied to the post-fixation image by creating a mask, and the mean fluorescence intensity (MFI) of each cell before and after fixation was collected. Data were expressed as a ratio of the post-fixation MFI relative to the live cell MFI (percent “fluorescence retention”) and analyzed for significance by one-way ANOVA with multiple comparisons.

Fluorescence Retention, proExM

Protein-retention expansion microscopy (proExM) was performed in a similar manner as the FP screening method from (Tillberg et al., 2016), with stock reagents and buffers prepared as described.

Note: in our opinion, the following process for comparing fluorescence retention between FPs after proExM is reproducible and ultimately effective, but we caution that it is very labor-intensive due to the fragility of the 16-chamber glass slides, the number of replicates necessary (at least 2 hydrogels per FP from separate preps), and the time required to properly transfer and position the shrunken hydrogels for post-expansion imaging (subjectively, we would consider expansion of mouse coronal sections to be much easier due to size and morphological landmarks). The 16-chamber format is most useful when comparing many FPs together to address engineering questions, but otherwise, cells can be grown on Matrigel- or fibronectin-coated No. 1 cover glass, and gelation chambers can be constructed using stacked coverslips more easily. Whatever the culture format, for those planning to reproduce this method, we recommend closely following the cultured cell protocol that is articulated and illustrated by Asano et al. (Asano et al., 2018).

To summarize the protocol, 16-chamber No. 1 coverglass slides (Grace Bio-Labs, #GBL112358) were coated with Matrigel (Corning, #356235) to improve attachment, and HeLa cells were passaged to reach ~60-80% confluency at the time of transfection, approximately 24 hr later. Cells were transfected using the recommended conditions for 96-well format with Turbofect transfection reagent (Thermo Fisher) and 0.2 μg H2B-[FP] DNA, for nuclear localization. After overnight expression, cells were fixed using room temperature (RT) 4% PFA in PBS, pH 7.4 (Gibco), at RT for 10-15 min. Afterward, the cells were washed for 5 min with 0.1 M glycine in PBS to quench fixation, followed by two washes with PBS for 5 min each. The 16-chamber slide was then imaged using a Keyence BZ-X700 All-in-One Fluorescence Microscope equipped with a PlanFluor DL 4× 0.13/16.50 mm PhL air objective, standard GFP filter cube, and computer-controlled motorized stage. After manually focusing each well, 5×5 tile scans of 640×480 px (3×3 binning) resolution were acquired in 8-bit TIFF format under imaging conditions specific to each FP to provide optimal exposure while minimizing oversaturation. The imaging settings for each well were recorded.

After acquiring the pre-expansion images of post-fixed cells, samples were treated with acryloyl-X (AcX) (Thermo Fisher. #A20770) in PBS, pH 7.4, overnight at RT while protected from light. The next day, AcX was removed using a micropipette, and samples were washed 3 times for 5 min each using PBS. Ideally, the same side of the well should be used for every pipetting step to minimize loss of cells. Next, the slide was placed on a clean benchtop, with the experimenter seated low, facing the 16-chamber slide at approximately eye level.

The plastic silo chambers that are adhered to the upper silicone spacer were separated from the lower silicone gasket (which itself is adhered to the coverslip surface), by looping dental floss carefully between the silicone spacer and silicone gasket f om one end of the slide, all the way through and out the other. Alternating between gently pulling from one end of the floss at a time (like reins) with one hand while delicately holding the slide in place with the other hand (without pressing the slide against the benchtop), can be helpful to reduce the risk of coverslip breakage. After the floss has been passed through, the two gaskets usually remain in their original positions while PBS has seeped between the narrow gaps opened by the dental floss to weaken the adhesive. This way, the chamber and silicone spacer together can be separated from the lower gasket carefully using fingertips or forceps to lift the plastic chamber/space gently, beginning from one end of the slide to the other, taking great care to avoid bending the brittle No. 1 coverslip. To serve as a reservoir for the gelling step, the lower silicone gasket must remain behind, attached to the coverslip on which the slides were grown. Since PBS will have leaked out, as mentioned, fresh PBS should be added swiftly to any empty wells to prevent them from drying out (a P20 micropipette set to 20 μL, with gentle pipetting against the side of the gasket reservoir, is useful for this). Alternatively, Grace Bio-Labs offers a removal tool, but we have had far greater success using the dental floss.

The coverslip on which the cells are grown now has only the lower black silicone gasket attached to it. This slide was placed inside a clean, dry, 15 cm culture plate with a 1 μL drop of water beneath it to keep it in place by surface tension. An aluminum block was placed level on ice and the 15 cm plate with the slide inside was placed on the block. The condensation from the block kept the 15 cm plate in place through surface tension. The 15 cm plate provides an enclosure for the slide during gelling and helps transfer it more easily to the incubator. Gelling solution was prepared on ice and distributed into the wells as described, after PBS removal (Asano et al., 2018), the 15 cm plate lid was placed back on the plate, and the plate was then carried level to a 37° C. dry incubator (normoxic; not a tissue culture incubator) and the gel was polymerized for 60-90 min.

After gelling, the thick parafilm-coated No. 2 slide glass lid that was placed atop the gasket was removed. The black silicone gasket was very carefully and slowly peeled off using blunt forceps, taking the greatest care not to bend the coverslip to any degree. The glass coverslip was then cut using a diamond scribe, with good practice and eye protection, to separate the coverslip into sections of individual gels, each still attached to a square of glass as described (Asano et al., 2018). Thoughtful planning is necessary for maintaining sample order when the slide is divided, or else the gels cannot be distinguished.

Digestion buffer was prepared and supplemented with proteinase K (New England Biolabs, #P8107S), dispensed into 12-well plate, and the gels were immersed, with their cut glass fragment still attached, into the solution overnight at RT. The next day, the glass was carefully removed with thin forceps as described by Asano et al., with the gels remaining in the 12-well plate (Asano et al., 2018). Digestion buffer was removed using a P1000 micropipette or 5 mL serological pipette, taking care not to break the semi-transparent gels. The gels were then washed 3-4 times, ~20 min per wash, using PBS to semi-expand them. An orbital shaker on a slow setting is optional and can help facilitate diffusion. Semi-expanded gels were then shrunk back (to ~1.5× original size) using the same wash step timing using shrinking solution (1 M NaCl and 60 mM MgCl₂ in water) instead of PBS.

The shrunken gels were then imaged that same day using the same microscope settings that were recorded for each well from the pre-expanded images. When possible, the original orientation was maintained. Gels were imaged in a No. 1 thickness empty glass-bottom 6-well plate or in a MatTek 35 mm glass-bottom plate with a few microliters of shrinking solution added to help adhere the gels by surface tension. Gels become very sticky when dry and should be kept moist. Alternatively, the gels may be fully submerged in shrinking buffer if they can remain flat against the plate. Gels were flipped so that the cell-side would face down against the glass (determined by focusing). A metal spatula bent at the end was useful for transferring the gels from the 12-well plate into the glass-bottom imaging vessel, with practice.

The pre- and post-expansion tile scan image sets for each FP were stitched using Keyence BZ-X Analyzer software to produce a 2428×1821 px (3×3 binning) 8-bit image. Post-expansion images of the shrunken gels were registered by unwarping and aligning by hand in Adobe Photoshop to the pre-expansion image for each pair using the Screen layer blending option and transform tool until the images overlapped. The unwarped post-expansion image was saved as a separate layer and exported as the same file type without compression or further modification. Image pairs were thresholded identically in ImageJ, and cells with measurable fluorescence and positive overlap in both the pre- and post-expansion images were further analyzed. Oversaturated cells were not used. The mean fluorescence intensity (MFI) of individual cells after expansion were expressed as a percentage relative to the pre-expansion MFI for that cell.

### Antibody Compatibility

Antibody compatibility images were acquired as described (Campbell et al., 2020). Briefly, HEK293T cells were transfected with pcDNA3.1-FP for cytosolic expression and were fixed with room temperature (RT) 4% PFA before immunostaining using primary antibodies: Gt a GFP polyclonal, Abcam #ab6673; Gt a GFP polyclonal, Novus #NB1001770; Ms a GFP monoclonal, Invitrogen #A-11120). The secondary antibody Dk a Gt IgG Alexa 555 (Invitrogen #A-21432) was applied, followed by DAPI to label nuclei. Cells were imaged on a Nikon Eclipse 80i microscope after mounting on slides.

### Confocal Photobleaching

Photobleaching experiments were performed as described (Campbell et al., 2020) using a Zeiss LSM 880 confocal microscope equipped with a Plan-Apochromat 40×/1.3 Oil DIC UVVIS-IR objective. Live HeLa cells in phenol red-free media were transfected with 0.2 μg H2B-FP plasmid DNA using Turbofect (Thermo Fisher). Pinhole was set to 1 Airy unit (“0.9-μm section”), scan time 316 ms, pixel size 0.83 μm, pixel dwell 4.12 is, 256×256-px frames, and 12-bit depth. Laser power was measured at the objective with a Thorlabs PMD100 power meter equipped with S130VC photodetector (Thorlabs) and was initially set to the minimum power necessary to identify suitable regions of the live HeLa cell cultures for bleaching.

To initiate bleaching, power was raised to 147 μW from the blue diode (LSS FPs), or 18.3 μW (GFPs) or 9.8 μW (YFPs) from the argon-ion laser. Time series images were collected in the 490- to 650-nm emission range using ZEN acquisition software (Zeiss). Uniformly fluorescent nuclei were selected for analysis. Mitotic cells with bright and condensed/punctate nucleoli were excluded. Scaling of the initial emission rate to 1,000 photons st molecule' at t=0 s was performed as described to produce the photobleaching plots (Shaner et al., 2008).

### Cellular Brightness and Protein Solubility

The brightness of human cells expressing transfected FPs (“cellular brightness”) was obtained using the same P2A plasmid co-expression template and methods as described (Campbell et al., 2020). Bacterial brightness and protein solubility measurements were performed as described (Campbell et al., 2020). Briefly, bacteria were grown overnight in LB broth supplemented with 100 μg/mL ampicillin (“media”). The following day, cultures were adjusted to the identical optical density (OD₆₀₀=0.6) and passaged at a 1:100 v/v ratio into fresh media supplemented with 0.2% arabinose to induce expression. After overnight growth at 37° C., 100 μL culture from each replicate was dispensed into a 96-well optical plate (Corning), and fluorescence measurements were acquired using a plate reader with λₑₓ/λₑₘ=495/525 nm. Data were background subtracted and normalized to eGFP. The remaining cultures were pelleted and frozen at −80° C. for the protein solubility experiment.

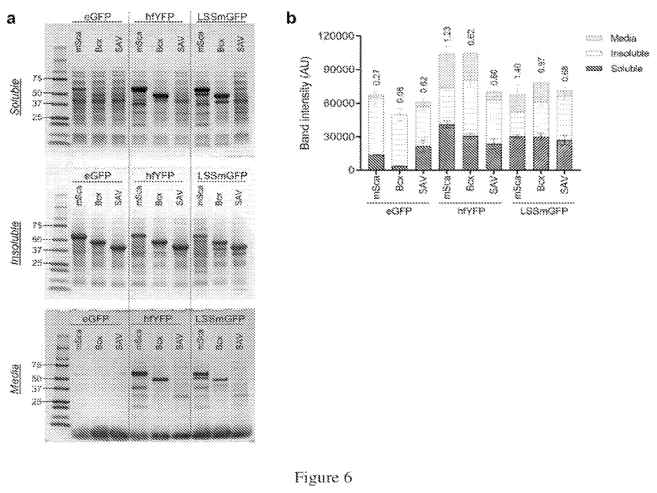

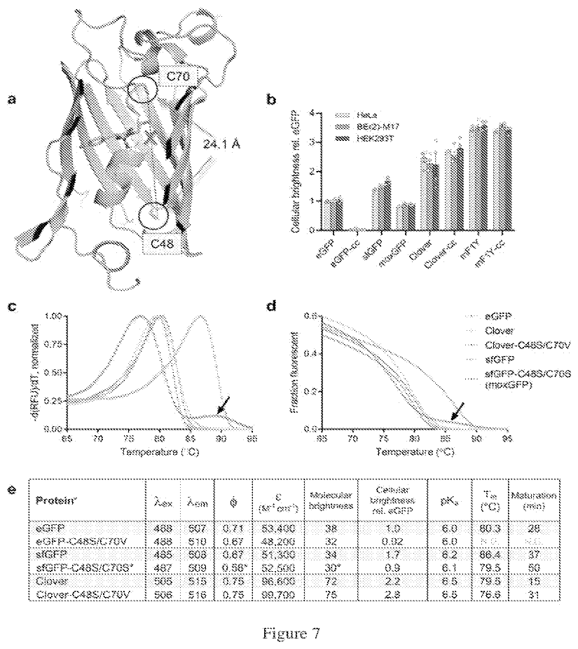

To obtain soluble protein, pellets were resuspended in PBS and lysed by freeze-thaw cycling between 37° C. water and liquid nitrogen, followed by sonication on ice and high-speed centrifugation at 4° C. to obtain the soluble fraction. The insoluble pellet was washed with ice-cold PBS, and this new supernatant containing residual soluble protein was discarded. The washed insoluble pellet was resuspended in ice-cold PBS and homogenized by brief sonication to yield in the insoluble fraction. Total protein concentration of the soluble and insoluble fractions was determined using the BCA Protein Assay (BCA) kit (Pierce) with BSA standard curve. Samples were adjusted to matching protein concentration and boiled for 10 min at 100° C. in the presence of SDS-containing loading dye supplemented with 0.2 M DTT. Samples were analyzed by SDS-PAGE on 4-15% Tris-Glycine gels (Bio-Rad), and the 27 kDa band representing the FP monomer was quantified using densitometry in ImageJ and normalized to the eGFP value. For the protein fusions shown in FIG. 5G and FIG. 6, the 57 kDa, 51 kDa, and 44 kDa bands were quantified for fusions of FPs with mScarlet-I, Bcx, and SAV, respectively.

### Correlative Light and Electron Microscopy (CLEM) Sample Preparation and Imaging Steps

### Cell-Free OsO4 Resistance Assay

OsO₄ dose-response curves were producing using an established protocol (Paez-Segala et al., 2015). Briefly, purified fluorescent proteins were dialyzed into 50 mM sodium phosphate buffer, pH 7.4, and normalized by concentration at A₂₈₀. A solution of 4% (w/v) OsO₄ (Electron Microscopy Sciences) was diluted to a final concentration of 1% and serially diluted in 96-well black clear-bottom optical plates (Greiner). Pure protein was added to the wells to a final concentration of 1 μM, and the plate was immediately sealed with optical adhesive (Bio-Rad). After 10 min incubation at room temperature, kinetic scans of FPs in OsO₄ solution were performed over a 10 min period at 1 min intervals using both the green (ex/em, 480/515 nm; bandwidth, 5 nm/5 nm) and yellow (ex/em, 493/517 nm; bandwidth, 5 nm/5 nm) channels. All measurements were performed with identical fluorometer settings.

### Fixation and Embedding for the Cellular OsO₄ Fluorescence Retention Experiment

The protocol for fixation and embedding from (Paez-Segala et al., 2015) was followed, with some modifications. HEK293 cells (ATCC) were maintained in Eagle's Minimum Essential Medium (DMEM), containing 1 mM sodium pyruvate, 4 mM L-glutamine, 4.5 g/mL glucose, and 1.5 g/mL sodium bicarbonate. Complete growth medium was prepared by addition of fetal bovine serum to 10% (w/v) final concentration.

To induce eGFP, mGreenLantern, and mhYFP cytosolic expression, cells were co-transduced with adeno-associated virus (AAV) particles with FP expression under control of a Cre-dependent CAG promoter (Addgene) along with AAV-CAG-Cre virus to induce expression. Approximately 2×106 cells were seeded on 100 mm plates for harvest and fixation. For live fluorescence measurements, ~8×10⁵ live HEK293 cells were seeded on 35 mm glass-bottom dishes (MatTek).

Brightness quantitation of the live cells before and after aldehyde/OsO₄ processing were performed using a laser-scanning confocal microscope (Zeiss LSM 880) with the following settings: 488 nm laser (0.5% power); 10× objective; emission range 495-560 nm; GaAsP detector gain of 715 V. Imaging settings for mhYFP used the 488 nm laser at 0.45% power; 10× objective; emission range 400-700 nm detection, and 600 V detector voltage. Images were quantified using Fiji (ImageJ). For background correction, the mean fluorescence intensity of 10 fields of view (FOVs) containing non-fluorescent cells were subtracted from each of 10 regions of interest (ROIs) containing the live fluorescent cells to be quantified.

After imaging the live cells, cultures were fixed with CLEM fixative (4% (w/v) paraformaldehyde (PFA)+0.2% (w/v) glutaraldehyde) (Electron Microscopy Sciences) in 100 mM phosphate buffer (PB) and then harvested in 10% bovine serum albumin (BSA) using a rubber spatula. Cells were then mixed with a 2% (w/v) agarose solution and pelleted. After the agarose solidified, it was cut using a scalpel into squares and submerged in a 1% (w/v) solution of OsO₄ for 1 hr at room temperature (RT).

Osmicated pellets in agarose were then embedded in optimal cutting temperature (OCT) frozen tissue specimen medium (Fisher Scientific, #23-730-571). 8-10 mm sections of the osmicated pellets were cut on a cryotome (Leica), placed on a glass slide, and mounted with antifade mounting media (Invitrogen). Images of the mounted cells were then captured using the same imaging settings as described for the live cells.

### OsO₄-Resistant Labels, High-Pressure Freezing, and Freeze Substitution

Live fluorescent cells seeded in 35 mm tissue culture dishes were fixed in CLEM fixative, harvested with a 20% (w/v) BSA solution, and assembled into the high pressure freezing (HPF) specimen carrier (Type A, 0.1/0.2 mm; Type B, flat: TechnoTrade). The carrier assembly was then introduced into the sample holder and frozen in the HPF machine per manufacturer's instructions (Wohlwend HPF Compact 01 High-pressure freezer; Techno Trade). Cell tissue was then freeze-substituted, osmicated with a 1% OsO₄ (w/v) solution, dehydrated in 100% (w/v) acetone, and embedded in Lowicryl HM20 resin (Electron Microscopy Sciences). After the resin was polymerized using UV lamp exposure, plastic blocks were cut into 100 μm sections for both Toluidine Blue staining and fluorescence visualization. Cells were mounted on glass slides using anti-fade mounting medium (Invitrogen).

### Crystallography

Fluorescent proteins were crystallized using the hanging drop vapor diffusion method with Hampton Research VDX 24-well plates. 2 μL protein (20-40 mg/mL) was gently pipetted into 2 μL reservoir solution on 22 mm diameter siliconized cover glass, sealed with grease atop a well containing 0.5 mL reservoir solution, and the plates were stored at 18° C. In our hands, the most favorable starting point for avFP crystallization performed this way has been solutions of 0.1 M Tris-HCl pH 8.5, 25-30% PEG 3350 or PEG 4000, 25-100 mM MgCl₂. The most effective PEGs in our hands were PEG 3350, PEG 4000, and PEG 8000. Likewise, the most effective salts were MgCl₂, Li₂SO₄, and sodium acetate, in descending order. Screening primarily consisted of optimization around the mentioned conditions with 0.1 M Tris-HCl, pH 8.5, and 18° C. storage in the dark.

hfYFP formed large, smooth, fluorescent yellow crystals after 1 wk in a solution of 0.1 M Tris-HCl pH 8.5, 25% PEG 3350, 0.2 M sodium acetate. The crystals matured for another 1 wk before they were looped out, cryoprotected using the same the reservoir solution (“mother liquor”) supplemented with 10% ethylene glycol, and flash frozen in liquid nitrogen. mhYFP formed crystals after 1 wk equilibration against reservoir solution consisting of 0.1 M Tris-HCl pH 8.5, 25% PEG 3350, 25 mM MgCl₂. The solution was a good cryo-condition and did not require supplementation. The mhYFP crystals were looped out and gently “washed” in a 2 μL droplet of reservoir solution (“mother liquor”) that was pipetted onto cover glass and handled within a humidified environment to minimize evaporation of the droplet, before flash freezing in liquid nitrogen. FOLD6 crystallized after 1 month in 0.1 M Tris-HCl pH 8.5, 30% PEG 4000, 100 mM MgCl₂. The crystals began to emerge from phase-separated green protein globules that coalesced and gradually transitioned from a semi-solid state into the final crystal form over a period of 1 month, after which they were looped out and frozen.

Note that we are treating mfoxYtiPLM as “mhYFP” in this section, although technically its V206K mutant is “mhYFP” (Table 1). We're indicating this here for accuracy and completeness, although previous studies have shown no significant structural differences from common position 206 substitutions apart from side chain identity (i.e. Ala, Lys, Val) (Goedhart et al., 2012; Von Stetten et al., 2012).

TABLE 1
Melting temperature and sequences of mutants
generated in this study, relative to Clover.

| Clover^{a} → | I167 | A206 | E124 | G232 | K101 | T105 | C48 | C70 | L221 | F223 |
|---|---|---|---|---|---|---|---|---|---|---|
| FOLD1 | T | — | — | — | — | — | — | — | — | — |
| mFOLD1 | T | K | — | — | — | — | — | — | — | — |
| mF1Y | T | K | — | — | — | — | — | — | — | — |
| mF1VPK | T | K | V | — | — | — | — | — | — | — |
| F2B | T | V | V | D | — | — | — | — | — | — |
| mF2B | T | V | V | D | — | — | — | — | K | R |
| mF2BK | T | V | V | D | — | — | — | — | K | R |
| mF2BK-ΔG | T | V | V | D | — | — | — | — | K | R |
| mGreenLantern^{b} | T | K | V | D | — | — | — | — | K | R |
| mF2BKK (DMN) | T | K | V | D | — | — | — | — | K | R |
| mF2B2 | T | V | V | D | — | — | — | — | K | R |
| F3C | T | V | V | D | E | Y | — | — | — | — |
| F3CP | T | V | V | D | E | Y | — | — | — | — |
| mF3CP | T | V | V | D | E | Y | — | — | K | R |
| mF3CPK | T | V | V | D | E | Y | — | — | K | R |
| Clover- | — | — | — | — | — | — | S | V | — | — |
| C48S/C70V | | | | | | | | | | |
| FOLD4 | T | V | V | D | E | Y | S | V | — | — |
| mFOLD4 | T | V | V | D | E | Y | S | V | K | — |
| mF4Y | T | V | V | D | E | Y | S | V | K | — |
| mF4Y-SR | T | V | V | D | E | Y | S | V | K | — |
| mF4Y-RK | T | K | V | D | E | Y | S | V | K | — |
| mF4Y-RKH | T | K | V | D | E | Y | S | V | K | — |
| F4P | T | V | V | D | E | Y | S | V | — | — |
| mF4P | T | V | V | D | E | Y | S | V | K | — |
| mF4P-ti | — | V | V | D | E | Y | S | V | K | — |
| FOLD6 | T | V | V | D | E | Y | S | V | — | — |
| FOLD6-HL | T | V | V | D | E | Y | S | V | — | — |
| mfoxY | T | V | V | D | E | Y | S | V | K | R |
| mfoxYY | T | V | V | D | E | Y | S | V | K | R |
| nFOLD7 | T | V | V | D | E | Y | — | — | K | R |
| Hyperfolder | — | V | V | D | E | Y | S | V | K | R |
| YFP^{c} | | | | | | | | | | |
| mfoxY-Y203F | — | V | V | D | E | Y | S | V | K | R |
| mhYFP-K | — | K | V | D | E | Y | S | V | K | R |
| mfoxYtiPLM | — | V | V | D | E | Y | S | V | K | R |
| mhYFP^{d} | — | K | V | D | E | Y | S | V | K | R |
| mfoxYtiPMKH | — | K | V | D | E | Y | S | V | K | R |
| mfoxYStr | T | V | V | D | E | Y | S | V | K | R |
| mfoxYStr-K | T | K | V | D | E | Y | S | V | K | R |
| mfoxYY3Str-K | — | K | V | D | E | Y | S | V | K | R |
| mfoxYY3Str | — | V | V | D | E | Y | S | V | K | R |
| mfoxYY3Str- | — | V | V | D | E | Y | S | V | K | R |
| SR-ti | | | | | | | | | | |

| Clover^{a} → | D234 | S147 | N149 | H203 | V68 | A69 | F46 | Other | Tₘ |
|---|---|---|---|---|---|---|---|---|---|
| FOLD1 | — | — | — | — | — | — | — | — | 87.6 |
| mFOLD1 | — | — | — | — | — | — | — | — | N.D. |
| mF1Y | — | — | Y | — | — | — | — | — | 84.9 |
| mF1VPK | — | P | K | — | — | — | — | — | N.D. |
| F2B | — | — | — | — | — | — | — | — | N.D. |
| mF2B | — | — | — | — | — | — | — | — | N.D. |
| mF2BK | — | — | K | — | — | — | — | — | 87.0 |
| mF2BK-ΔG | — | — | K | — | — | — | — | ΔG4 | 92.8 |
| mGreenLantern^{b} | — | — | K | — | — | — | — | — | 87.2 |
| mF2BKK (DMN) | N | — | K | — | — | — | — | — | N.D. |
| mF2B2 | N | — | — | — | — | — | — | — | N.D. |
| F3C | N | P | — | — | — | — | — | — | N.D. |
| F3CP | N | P | — | — | — | — | — | — | N.D. |
| mF3CP | — | P | — | — | — | — | — | — | N.D. |
| mF3CPK | — | — | K | — | — | — | — | — | 86.5 |
| Clover- | — | — | — | — | — | — | — | — | 76.6^{e} |
| C48S/C70V | | | | | | | | | |
| FOLD4 | — | — | — | — | — | — | — | — | 86.2 |
| mFOLD4 | — | — | — | — | — | — | — | — | 84.3 |
| mF4Y | — | — | Y | — | — | — | — | — | 85.2 |
| mF4Y-SR | — | R | — | — | — | — | — | — | 84.3 |
| mF4Y-RK | — | R | — | — | — | — | — | — | 84.2 |
| mF4Y-RKH | — | R | — | — | — | — | — | E222H | 85.6^{e} |
| F4P | — | P | — | — | — | — | — | — | 87.4 |
| mF4P | — | P | — | — | — | — | — | — | 87.3^{e} |
| mF4P-ti | — | P | — | — | — | — | — | — | 85.2 |
| FOLD6 | — | P | — | — | L | — | | — | 90.0 |
| FOLD6-HL | — | P | — | — | L | — | | H169L | 89.8 |
| mfoxY | N | — | — | Y | L | M | L | — | 92.8 |
| mfoxYY | N | — | Y | Y | L | M | L | — | N.D. |
| nFOLD7 | N | P | — | Y | L | M | L | — | 90.6 |
| Hyperfolder | N | — | — | Y | L | M | L | — | 94.2 |
| YFP^{c} | | | | | | | | | |
| mfoxY-Y203F | N | — | — | F | L | M | L | — | 94.5 |
| mhYFP-K | N | — | — | Y | L | M | L | — | 94.0 |
| mfoxYtiPLM | N | P | — | Y | L | M | L | L195M | 93.6 |
| mhYFP^{d} | N | P | — | Y | L | M | L | L195M | 92.8 |
| mfoxYtiPMKH | N | P | — | Y | L | M | L | L195M | 88.9 |
| | | | | | | | | E222H | |
| mfoxYStr | N | — | — | Y | L | M | L | YF^{f} | 83.5 |
| mfoxYStr-K | N | — | — | Y | L | M | L | YF^{f} | 84.1 |
| mfoxYY3Str-K | N | — | Y | Y | L | M | L | YF^{f} | N.D. |
| mfoxYY3Str | N | — | Y | Y | L | M | L | YF^{f} | 81.6 |
| mfoxYY3Str- | N | R | Y | Y | L | M | L | YF^{f} | 80.7 |
| SR-ti | | | | | | | | | |

^{b}mGreenLantern (mGL) = mF2BK-K (DMD).
^{c}Hyperfolder YFP (hfYFP) = mfoxYti.
^{d}mhYFP = hfYFP-S147P/L195M = mfoxYtiPLM-V206K = mfoxYtiPMK.
^{e}FPs that showed more than one melt peak are indicated. The secondary peak for Clover-C48S/C70V, mF4P, and mF4Y-RKH, appear at 88.9 °C, 96.7 °C, and 76.8 °C, respectively. See FIG. S1c-d and FIG. S3a-b for melt curves.
^{f}YF: F145Y/N146F.
N.D.: not determined.

### Crystallographic Data Collection, Processing, and Refinement

Crystallographic diffraction data were collected at GM/CA CAT 23-IDD of the Advanced Photon Source at Argonne National Laboratory with monochromatic x-rays of 1.033 angstroms at 100 K on a Dectris Pilatus3 6M HPC detector (Dectris Ltd., Switzerland) and processed with XDS (Kabsch, 2010). Structural homology model of each respective proteins was generated from the crystal structure of Clover (PDB: 5WJ2) (Campbell et al., 2018) using the online server Swiss-Model (Schwede et al., 2003). These homology models were used as a molecular replacement search model and yielded a solution with the program Phaser (McCoy et al., 2007). The initial molecular replacement solution was subjected to multiple rounds of maximum likelihood restrained refinement using PHENIX (Adams et al., 2010) and manual rebuilding with Coot (Emsley and Cowtan, 2004). Complete data collection and refinement statistics are provided in Table 4.

TABLE 4
Crystallographic data collection and refinement statistics

| Protein | hfYFP | mhYFP | FOLD6 |
|---|---|---|---|
| Resolution range | 27.39-1.74 | 27.28-1.63 | 29.63-1.21 |
| | (1.802-1.74) | (1.688-1.63) | (1.253-1.21) |
| Space group | C222₁ | C222₁ | P6₄ |
| Unit cell | 51.994 66.872 | 52.347 66.659 | 61.438 61.438 |
| | 143.308 90 90 90 | 142.465 90 90 90 | 112.38 90 90 120 |
| Total reflections | 78920 (8237) | 198902 (19649) | 306013 (2199) |
| Unique reflections | 25423 (2564) | 31489 (3070) | 64116 (1788) |
| Multiplicity | 3.1 (3.2) | 6.3 (6.3) | 4.8 (1.2) |
| Completeness (%) | 97.44 (99.77) | 97.66 (99.19) | 87.89 (24.56) |
| Mean I/sigma(I) | 8.58 (2.46) | 25.79 (4.36) | 18.36 (0.36) |
| Wilson B-factor | 29.43 | 24.84 | 16.31 |
| R-merge | 0.08541 (0.3248) | 0.03641 (0.3444) | 0.03744 (1.128) |
| R-meas | 0.1034 (0.3919) | 0.03978 (0.3754) | 0.04124 (1.552) |
| R-pim | 0.05718 (0.2156) | 0.01581 (0.1481) | 0.017 (1.06) |
| CC1/2 | 0.989 (0.873) | 0.999 (0.942) | 1 (0.29) |
| CC* | 0.997 (0.966) | 1 (0.985) | 1 (0.671) |
| Reflections used | 25423 (2564) | 30785 (3070) | 64115 (1789) |
| in refinement | | | |
| Reflections used | 1333 (134) | 1144 (114) | 2006 (59) |
| for R-free | | | |
| R-work | 0.2015 (0.3351) | 0.1503 (0.1923) | 0.1458 (0.3245) |
| R-free | 0.2344 (0.3473) | 0.1875 (0.2104) | 0.1710 (0.3412) |
| CC(work) | 0.955 (0.806) | 0.968 (0.899) | 0.975 (0.468) |
| CC(free) | 0.943 (0.716) | 0.948 (0.847) | 0.959 (0.433) |
| Number of non- | 2016 | 2061 | 2200 |
| hydrogen atoms | | | |
| macromolecules | 1789 | 1836 | 1903 |
| ligands | 55 | 19 | 19 |
| solvent | 172 | 206 | 278 |
| Protein residues | 227 | 227 | 229 |
| RMS(bonds) | 0.01 | 0.015 | 0.004 |
| RMS(angles) | 1.38 | 1.83 | 0.99 |
| Ramachandran | 97.75 | 98.65 | 98.66 |
| favored (%) | | | |
| Ramachandran | 2.25 | 1.35 | 1.34 |
| allowed (%) | | | |
| Ramachandran | 0 | 0 | 0 |
| outliers (%) | | | |
| Rotamer outliers (%) | 0 | 0.99 | 1.87 |
| Clashscore | 3.28 | 4.62 | 2.86 |
| Average B-factor | 33.67 | 32.9 | 23.11 |
| macromolecules | 32.95 | 32.01 | 21.51 |
| ligands | 37.34 | 21.17 | 14.82 |
| solvent | 39.97 | 41.92 | 34.61 |
| Number of TLS groups | | 1 | |

^{a} Statistics for the highest-resolution shell are shown in parentheses.

### Example 2. Library Development

We first created cysteine-free mutants of a variety of Aequorea victoria fluorescent proteins (avFPs) and compared their brightness, thermostability, and spectroscopic characteristics. Clover (Lam et al., 2012) tolerated cysteine substitution better than eGFP and sfGFP (FIGS. 7A-7E). Whereas the C48S/C70V mutations eliminated eGFP's fluorescence in cells, Clover-C48S/C70V was brighter than wild-type Clover, and sfGFP-C48S/C70S (moxGFP; (Costantini et al., 2015)) was almost 50% dimmer than sfGFP (FIG. 7E). The spectroscopic properties of each cysteine-substituted mutant were essentially unchanged (FIG. 7E), suggesting that the brightness in cells results from better expression, folding, maturation, and/or stability.

We expanded the library built on Clover-based mGreenLantern (mGL; Campbell et al., 2020) by introducing C48S/C70V substitutions, followed by combinations of folding mutations selected based on extensive analysis of structure-activity relationships (SARs) in avFPs. The unusually long persistence of mGL in guanidinium hydrochloride (GdnHCl) was one of the defining stability metrics that distinguished it from sfGFP and underlay its improved performance. In this vein, we screened FPs in kinetic unfolding assays and determined their melting temperatures (Tₘ) to identify mutants with improved thermodynamic stability. We compared individual and concerted folding mutations in this background in a generally stepwise manner (Table 1) (FIG. 8), relying on spectroscopic characterization (Table 2), thermostability measurements (FIGS. 9A-9B), FP brightness in live human cells (“cellular brightness”) (FIG. 9C), and GdnHCl stability (FIG. 9D) to identify trends that would guide the ensuing library design steps. Individual and concerted sets of mutations conferring unique properties could often be transferred into other templates.

TABLE 2
Biochemical characterization of selected FPs and their brightness in human cells.

| | | | | | | | | Cellular | | | | |
|---|---|---|---|---|---|---|---|---|---|---|---|---|
| | | | | | ε | | Molecular | brightness^{c} | | | Tₘ | Maturation |
| Protein | λₑₓ | λₑₘ | φ | ± | (M⁻¹ cm⁻¹) | ± | brightness^{a} | rel. eGFP | pKₐ | ± | (°C) | (min) |
| eGFP | 488 | 507 | 0.71 | 0.010 | 53,400 | 700 | 38 | 1.1 | 6.0 | 0.19 | 80.3 | 28 |
| eGFP- | 488 | 510 | 0.68 | 0.041 | 48,200 | 1,500 | 32 | 0.0 | 6.0 | 0.17 | N.D. | N.D. |
| C48S/C70V | | | | | | | | | | | | |
| Superfolder | 485 | 508 | 0.67 | 0.023 | 51,300 | 1,400 | 34 | 1.7 | 6.2 | 0.12 | 86.4 | 37 |
| GFP | | | | | | | | | | | | |
| moxGFP^{b} | 487 | 509 | 0.58^{d} | N/A | 52,500 | 3,200 | 30^{e} | 0.9 | 6.1 | 0.12 | 79.5 | 50 |
| Clover | 505 | 515 | 0.75 | 0.048 | 96,600 | 1,400 | 72 | 1.8 | 6.5 | 0.21 | 79.5 | 15 |
| Clover- | 506 | 516 | 0.75 | 0.029 | 99,700 | 6,100 | 75 | 2.4 | 6.5 | 0.17 | 76.6 | 31 |
| C48S/C70V | | | | | | | | | | | | |
| mClover3 | 506 | 516 | 0.80 | 0.015 | 100,000 | 2,800 | 80 | 1.6 | 6.1 | 0.02 | 80.1 | 37 |
| mNeonGreen | 506 | 516 | 0.78 | 0.037 | 115,400 | 2,200 | 90 | 4.1 | 5.6 | 0.25 | 68.0 | 18 |
| eYFP | 513 | 527 | 0.60 | 0.055 | 94,100 | 2,800 | 58 | 1.9 | 6.8 | 0.29 | 72.9 | >37 |
| FOLD1 | 503 | 514 | 0.70 | 0.016 | 99,700 | 2,000 | 70 | 3.4 | 5.8 | 0.14 | 87.6 | N.D. |
| mF1Y | 503 | 517 | N.D. | N.D. | 104,600 | 570 | 77 | 3.9 | N.D. | N.D. | 84.9 | 7 |
| mF1VPK | 503 | 513 | 0.75 | 0.013 | 107,700 | 3,200 | 81 | 3.2 | N.D. | N.D. | N.D. | N.D. |
| mF2BK (DMD) | 503 | 514 | 0.71 | 0.029 | 101,300 | 2,700 | 72 | 3.1 | 5.6 | 0.16 | 87.0 | 9 |
| mGreenLantern | 503 | 514 | 0.72 | 0.017 | 101,800 | 630 | 74 | 7.4 | 5.6 | 0.01 | 87.2 | 14 |
| mF3CPK | 503 | 513 | 0.72 | 0.014 | 100,700 | 4,500 | 73 | 2.4 | 5.9 | 0.27 | 86.5 | N.D. |
| FOLD4 | 504 | 513 | 0.69 | 0.015 | 108,200 | 3,300 | 75 | 3.5 | 6.0 | 0.09 | 86.2 | 16 |
| F4P | 503 | 513 | 0.76 | 0.017 | N.D. | N.D. | N.D. | 2.9 | N.D. | N.D. | 87.4 | N.D. |
| mF4P | 504 | 516 | 0.75 | 0.032 | 120,500 | 3,600 | 93 | 3.0 | 5.9 | 0.20 | 87.3 | 12 |
| mF4Pti | 506 | 516 | 0.77 | 0.041 | 118,200 | 5,300 | 91 | 1.7 | 6.3 | 0.08 | 85.2 | 25 |
| m4FY | 504 | 516 | 0.80 | 0.038 | 100,000 | 2,000 | 83 | 3.1 | N.D. | N.D. | 85.2 | 16 |
| mF4Y-SR | 505 | 516 | 0.73 | 0.024 | 107,200 | 3,500 | 81 | 3.5 | 5.6 | 0.02 | 84.3 | 6 |
| mF4Y-RKH | 497 | 513 | 0.56 | 0.022 | N.D. | N.D. | N.D. | N.D. | 6.4 | 0.20 | 85.6 | N.D. |
| FOLD6 | 505 | 515 | 0.73 | 0.032 | 118,200 | 1,100 | 86 | 2.3 | 5.9 | 0.21 | 90.0 | 25 |
| mF6-H169L | 505 | 515 | 0.75 | 0.046 | 108,900 | 5,300 | 84 | 1.1 | 6.7 | 0.23 | 89.8 | N.D. |
| mfoxY | 512 | 529 | 0.51 | 0.006 | 110,000 | 3,200 | 56 | 2.7 | 5.5 | 0.04 | 92.8 | 25 |
| Hyperfolder | 514 | 529 | 0.60 | 0.020 | 119,500 | 1,400 | 74 | 2.6 | 5.6 | 0.23 | 94.2 | 21 |
| YFP | | | | | | | | | | | | |
| mHyperfolder | 515 | 529 | 0.62 | 0.019 | 124,000 | 3,200 | 77 | 2.5 | 5.7 | 0.02 | 92.8 | 27 |
| YFP | | | | | | | | | | | | |
| mfoxYtiPMKH | 509 | 514 | N.D. | N.D. | N.D. | N.D. | N.D. | N.D. | 8.5 | 0.02 | 88.9 | N.D. |
| mfoxY-Y203F | 508 | 520 | 0.72 | 0.035 | 120,000 | 3,400 | 89 | 1.1 | 5.6 | 0.03 | 94.5 | N.D. |

Note:
except for moxGFP, eGFP-C48S/C70V, and Clover-C48S/C70V, data above the “FOLD4” row are cited from Campbell et al., PNAS, 2020, for the purpose of structure-activity comparison.
All data in the table were generated in our lab in this study unless otherwise stated.
φ = quantum yield; ε = extinction coefficient at peak absorbance. N.D.: not determined.
^{a}Molecular brightness = (φ × ε)/10³.
^{b}moxGFP is human codon-optimized sfGFP-C48S/C70V; we have re-characterized it for this study.
^{c}In BE(2)-M17 cells. See FIG. S3c for results from additional human cell lines.
^{d}The quantum yield of moxGFP is cited from Costantini et al., Nat. Comm., 2015. All other data in the table were generated in our lab.
^{e}Molecular brightness of moxGFP was determined using our EC value and the cited QY value.
±: s.d.

The most stable mGL variant generated from this effort, hyperfolder YFP (hfYFP), has a melting temperature of 94.2° C., an improvement of 21° C. above eYFP's Tₘ and 8° C. above sfGFP's. The majority of mGL variants showed superior stability in 6.3 M GdnHCl solutions relative to sfGFP, persisting for over 1 hr before falling to half-initial fluorescence values. Intriguingly, rather than falling, hfYFP's fluorescence instead increased 50% upon exposure to GdnHCl 6.3 M and remained constant for over 10 hours, the full duration of the experiment (FIG. 9D). Cysteine-containing mutants such as sfGFP failed to refold after denaturation (FIG. 10A) unless reducing agent was present in solution (FIG. 10B), whereas the cysteineless variants fully refolded under all conditions.

### Example 3. Characterization

The spectral properties and performance of hyperfolder YFP (hfYFP) in cells (Table 5)—having similar excitation/emission maxima (FIG. 1A) but lacking eYFP's 405 nm absorbance peak (FIG. 1B)—make it an appealing replacement for eYFP. hfYFP exhibits faster chromophore maturation (FIG. 1C), fluorescence intensity in E. coli equivalent to mGL's and greater than all other GFPs/YFPs tested (FIG. 1D), 33% greater brightness than eYFP and 2.4-fold greater brightness than eGFP in three human cell lines (FIG. 1E), greater pH stability than eYFP (FIG. 1F), and insensitivity to chloride (FIG. 1G). The brighter bacterial fluorescence corresponded with improved soluble protein production (FIG. 11A). Only 50-60% of the total eGFP, mNeonGreen, eYFP, and sfGFP protein produced by E. coli appeared in the soluble fraction, compared to 68% of mGL and nearly 80% of hfYFP (FIG. 11B). Thus, improved folding efficiency and soluble protein production contribute to the superior brightness of hfYFP in bacteria.

TABLE 5
Spectroscopic characterization of FPs.

| | | | | | | Cell. | | | |
|---|---|---|---|---|---|---|---|---|---|
| | | | | ε | Mol. | bright. | | Maturation | Tₘ |
| Protein | λₑₓ | λₑₘ | φ | (M⁻¹ cm⁻¹) | bright.^{a} | (×EGFP)^{b} | pKₐ | (min) | (°C) |
| eGFP | 488 | 507 | 0.71 | 53,400 | 38 | 1.0 | 6.0 | 28 | 80.3 |
| Superfolder | 485 | 508 | 0.67 | 51,300 | 32 | 1.7 | 6.2 | 37 | 86.4 |
| GFP | | | | | | | | | |
| eYFP | 513 | 527 | 0.60 | 94,000 | 58 | 1.8 | 6.8 | 37^{c} | 72.9 |
| Hyperfolder | 514 | 529 | 0.60 | 119,500 | 72 | 2.6 | 5.6 | 21 | 94.2 |
| YFP | | | | | | | | | |
| mT- | 400 | 510 | 0.63 | 35,400 | 22 | N.D.^{d} | 5.1 | N.D. | 86.3 |
| Sapphire | | | | | | | | | |
| mAmetrine | 412 | 527 | 0.48 | 33,400 | 16 | N.D. | 6.3 | N.D. | 74.8 |
| LSSmGFP | 400 | 510 | 0.48 | 36,900 | 18 | N.D. | 4.7 | N.D. | 84.8 |
| LSSA12 | 398 | 511 | 0.38 | 38,700 | 15 | N.D. | 4.6 | N.D. | 93.9 |

^{a}Molecular brightness = (φ × ε)/10³.
^{b}Mean fluorescence intensity of Hela cells expressing the FPs relative to eGFP (P2A system).
^{c}eYFP reaches its 50% half-maximal value in 37 min, after which point it shows a slow phase not observed in the other FPs.
^{d}N.D.: not determined.

All values in this table were determined experimentally in our lab for this study.

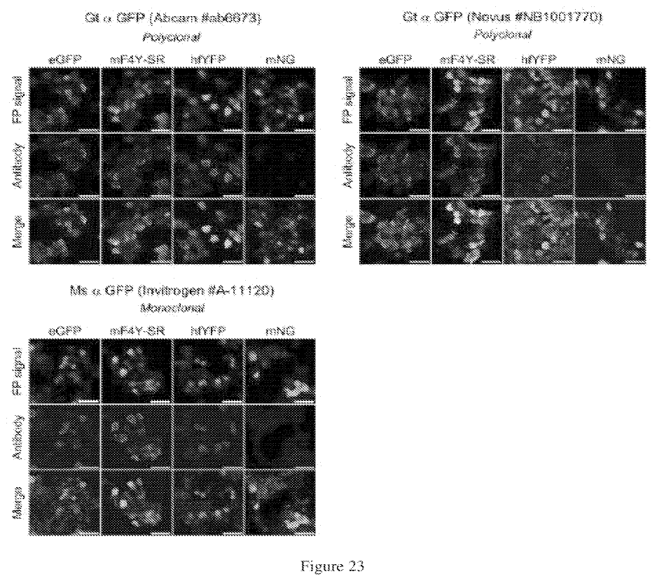

hfYFP tolerated deleterious mutations that rendered eGFP and even sfGFP almost entirely nonfunctional, suggesting that it will be a good template for mutagenesis and sensor engineering. hfYFP is compatible with antibodies designed for eGFP (FIG. 23), and cells transfected with nuclear localized hfYFP and mhYFP show healthy morphology. With photostability under laser-scanning confocal illumination approximately equal to mGL's (Campbell et al., 2020), hfYFP bleaches faster than Clover or eYFP, so care should be taken during intensive prolonged imaging. Compared to human codon-optimized moxGFP, a cysteine-free sfGFP mutant, hfYFP offers 2-fold greater molecular brightness, nearly 3-fold greater cellular brightness, improved acid resistance, much greater thermodynamic stability (Tₘ=94.2° C. vs. 79.5° C., respectively), 2-fold accelerated chromophore maturation rate (Table 2), and faster refolding (FIG. 10). moxGFP denatured immediately upon exposure to GdnHCl, whereas hfYFP never denatured (FIG. 9D).

Hyperfolder YFP localized properly upon fusion or targeting to common intracellular targets, including actin, tubulin, clathrin, endoplasmic reticulum, mitochondria, and nuclei (FIGS. 1H-1M), indicating that it should perform well in difficult fusions that demand monomeric fluorescent proteins. Monomeric hyperfolder YFP (mhYFP) eluted as a pure monomer by gel filtration chromatography (FIG. 12A) and scored as a monomer in the organized smooth endoplasmic reticulum (OSER) assay (FIG. 12B). hfYFP exhibited weak dimer properties in this assay, like Clover (FIG. 12C) and eGFP, both of which perform well in fusions. Altogether, mhYFP and hfYFP offer many advantages over eYFP that make them better choices for routine imaging.

### Example 4. Chemical Stability

We subjected hfYFP to a barrage of denaturing challenges and compared it to widely used FPs (eGFP, sfGFP, mClover3, mNconGreen, eYFP, and mGreenLantern) in each experiment. Consistent with previous experiments (Campbell et al., 2020), sfGFP, mClover3, mNeonGreen, and eYFP protein fully denatured within 10 min after dilution into buffered 6.3 M GdnHCl, whereas mGL remained above its half-initial fluorescence value until ~200 min. mGL fluorescence was still measurable after 12 hr, and the signal was above background levels for 24 hr (FIG. 2A). Intriguingly, instead of dimming, hfYFP grew 50% brighter in 6.3 M GdnHCl. When measured after 12, 24, and 48 hr, hfYFP fluorescence was unchanged relative to its value at 2 hr (FIG. 2A). Whereas sfGFP denatured instantly in 7 M GdnHCl, hfYFP persisted >3 months in the same solution at room temperature (FIG. 2A, quantification in Table 3).

TABLE 3
Quantification of chemical and thermal denaturation experiments from FIG. 2.

| | | | Kinetic | Equilibrium | Kinetic | Equilibrium | |
|---|---|---|---|---|---|---|---|
| | | 87 °C | unfolding, | unfolding, | unfolding, | unfolding, | NaOH |
| | Tₘ | persistence | GdnHCl | GdnHCl | GdnSCN | GdnSCN | persistence, |
| Protein | (°C) | t_{1/2} (min) | t_{1/2} (min) | C_{1/2} (M) | t_{1/2} (min) | C_{1/2} (M) | t_{1/2} (s) |
| Hyperfolder | 94.2 | 40.2 | ∞ | ^{c}∞ | 9.3 | 3.2 | 18 |
| YFP | | | | | | | |
| mhYFP^{b} | 92.8 | N.D. | N.D. | N.D. | 6.4 | 3.0 | 120 |
| FOLD6 | 90.0 | N.D. | N.D. | N.D. | N.D. | N.D. | 142 |
| mF4P | 87.3 | ^{b}3.1 | N.D. | N.D. | 0.1 | 2.2 | 84 |
| mGreen Lantern | 87.2 | 17.4 | 206.1 | 5.5 | 1.6 | 2.3 | 0 |
| Superfolder | 86.4 | 1.5 | 2.9 | 4.3 | 0 | 0.1 | 0 |
| GFP | | | | | | | |
| eGFP | 80.3 | 0 | <1 | 4.0 | 0 | 0.2 | 0 |
| mClover3 | 80.1 | 0 | 1.8 | 3.8 | 0 | 0.1 | 0 |
| eYFP | 72.9 | 0 | 2.4 | 1.0 | 0 | 0.1 | 1 |
| mNeonGreen | 68.0 | 0 | 9.2 | 2.0 | 0 | 0.8 | 0 |

^{a}Tm data here are as shown in Table 2 for the purpose of comparison (typical experimental error: ±2 °C).
∞ Hyperfolder YFP was stable in GdnHCl for at least 3 months at room temperature in 7M GdnHCl, pH 7.4. The solubility limit for GdnHCl in water is approximately 8M.
^{b}mF4P shows a double-exponential melting process with a fast phase t_{1/2} = 3.1 min and τ = 4.5 min during which 80% of the initial fluorescence is lost, followed by an extended slow phase (τ = 122 min) of t_{1/2} = 85 min, eventually intercepting hyperfolder YPP's mono-exponential curve at τ = 200 min and continuing with it for the rest of the process.
^{b}Monomeric hyperfolder YFP is hfYFP-S147P/L195M/V206K.
^{c}Fluorescence at the highest GdnHCl concentration of 6.3M was ~50% greater than the initial value in buffer without GdnHCl.

Likewise, in guanidinium thiocyanate (GdnSCN), a stronger denaturant than GdnHCl that has been studied in the context of sfGFP (Stepanenko et al., 2014), almost all FPs—including sfGFP—denatured instantly in 3.6 M concentration, while mGL and hfYFP did not (FIG. 2B). Instead, mGL and hfYFP persisted for 1.6 and 9.3 min before reaching their half-maximal intensity values along double- and mono-exponential decay trajectories, respectively, with hfYFP persisting over 40 min before its fluorescence fell below background levels (FIG. 2B).

Equilibrium unfolding experiments using GdnHCl confirmed the behavior of hfYFP in 6.3 M GdnHCl and demonstrated dose-dependent fluorescence intensity responses directly proportional to GdnHCI concentration—in marked contrast to the other FPs, which denatured rapidly after surpassing a critical GdnHCl concentration. Consistent with the kinetic unfolding data, hfYFP brightness in 6.3 M GdnHCl was approximately 50% greater than without GdnHCl (FIG. 2C). mGL and hfYFP also outperformed the other FPs in the more chaotropic GdnSCN solutions during equilibrium unfolding (FIG. 2D, Table 3). The data provide further evidence that hfYFP is stabilized by GdnHCl even at concentrations that rapidly denature common fluorescent proteins and almost all known proteins.

Next, we measured the length of the time that FPs could remain fluorescent in Tris-buffered solutions maintained at specific high temperatures (“isothermal melting”). Mirroring the GdnSCN denaturation trends (FIG. 2B), fluorescence quenching of mGL and hfYFP followed double- and mono-exponential decay trajectories with half-maximal time values (t_{1/2}) of 17.4 and 40.2 min, respectively, in a thermal cycler programmed to maintain 87° C. while fluorescence measurements were collected every minute (FIG. 2E). All other FPs fully denatured within 8 min, including sfGFP (t_{1/2}=1.5 min) (FIG. 2E) (Table 3).

A summary of relative intensity values at the 60 min timepoint for each of the six temperatures tested are presented in FIG. 2F, again showing similar rank-ordered stability trends: hfYFP was the most stable, followed by mGL and then by sfGFP. eYFP was only marginally fluorescent after 1 hr at just 67° C., and mNeonGreen could not withstand any condition for more than several minutes. Like eYFP, mNG instantly denatured at T≥80° C. (FIG. 13). Additional melting curve experiments with freshly purified proteins confirmed the absence of batch-to-batch response variability and faithfully reproduced Tₘ values recorded during the screening process (FIG. 2G). The melting temperature of hfYFP (Tₘ=94.2° C.) was approximately 20° C. and 14° C. greater eYFP's and eGFP's values, respectively. mGL and hfYFP can tolerate higher temperatures for much greater lengths of time than the other FPs.

When exposed to large molar excess quantities of hydrogen peroxide (H₂O₂), hfYFP withstood the greatest concentrations. mNeonGreen and mClover3 were again the least stable, and sfGFP was unimproved relative to eGFP (FIG. 2H). Altogether, the data demonstrate the superior stability of hfYFP against multiple chemical and thermal denaturing challenges.

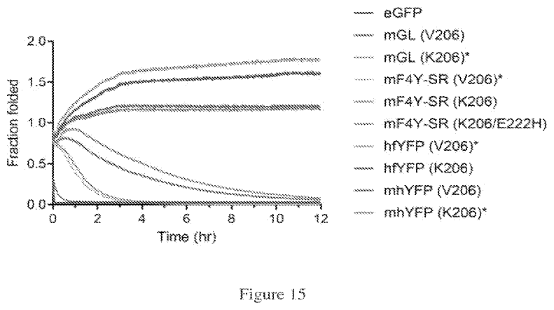

We next targeted β-strand 7, the most structurally heterogeneous region of avFPs (Baffour-Awuah et al., 2005; Goedhart et al., 2012), to engineer variants with further enhanced stability. Introducing the S147P mutation that improves thermostability in uvGFP yielded hyperfolder mutants with considerable resistance to 1 M sodium hydroxide (NaOH) solutions (pH≥13)—routinely used for extinction coefficient determination by alkali denaturation (FIG. 14). hfYFP-S147P/V206K/L195M behaved as a stronger monomer in the OSER assay than did hfYFP (FIGS. 12B-12C); the L195M mutation originated de novo. The V206K mutation (on β-strand 10) largely preserved GdnHCI stability in multiple mutants (FIG. 15). We named the hfYFP-S147P/V206K/L195M variant “monomeric hyperfolder YFP” (mhYFP). mhYFP further demonstrates that peculiar performance features can be structurally engineered into FPs without perturbing spectral properties (Table 2, FIG. 16).

### Example 5. Compatibility with Fixatives and Expansion Microscopy

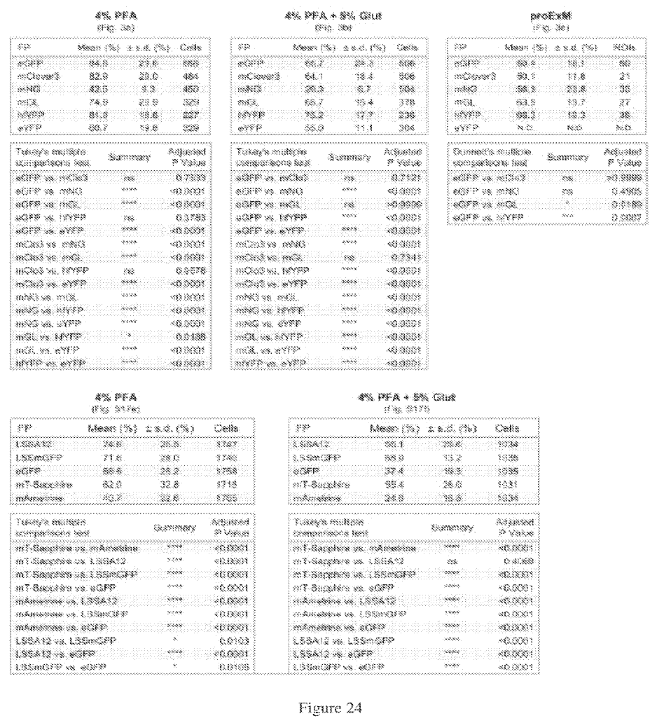

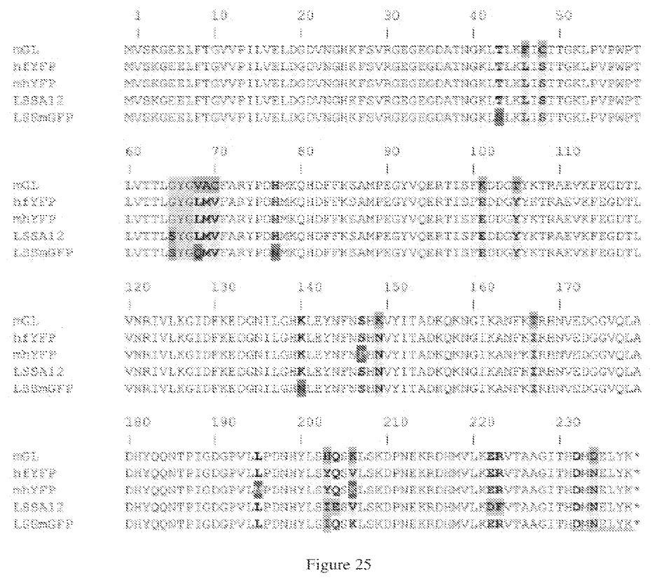

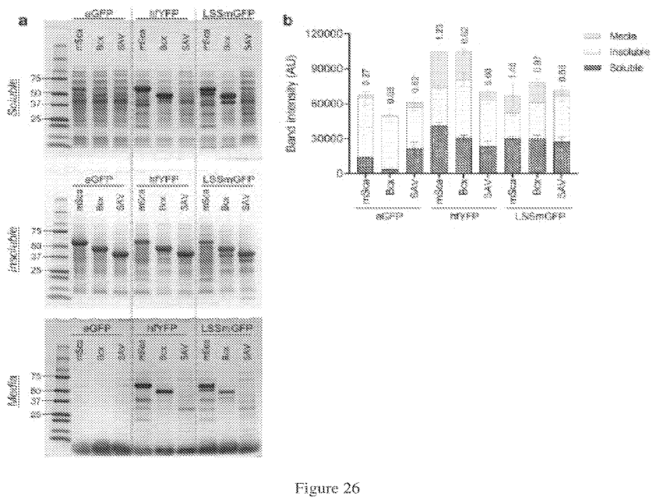

Paraformaldehyde (PFA) and glutaraldehyde (Glut) are the most common histological preservatives in biology (Richter et al., 2018), and an FP that can retain the greatest fluorescence in aldehyde fixatives would have considerable advantages in any downstream optical application. HEK293T cells expressing eGFP and hfYFP lost approximately 20% of their fluorescence after fixation with 4% PFA in phosphate buffered saline (PBS), pH 7.4. Interestingly, consistent with the thermal and chemical stability results, mNG and eYFP fared the worst against PFA, keeping only 42% and 60% of their original fluorescence, respectively (FIG. 3A). hfYFP retained 75% of its fluorescence after fixation with a 4% PFA+5% Glut solution compared to 65% for mGL and eGFP, and 29% for mNG (FIGS. 3B-3C) (FIG. 24). mhYFP is compatible with protein-retention expansion microscopy (proExM) (Tillberg et al., 2016) (FIG. 3D) and retains 16% more fluorescence than eGFP at the end of the process (FIG. 3E). The data highlight the diversity of responses to aldehyde fixatives between FPs and demonstrate the degree to which experiments are impacted by fluorescence quenching at the earliest stage of sample processing.

mhYFP and mGL retained greater fluorescence after protein-retention expansion microscopy (proExM) compared to other FPs (FIG. 3A). Cells were fixed with 4% paraformaldehyde (PFA) before confocal imaging; cellular fluorescence was compared after expanded hydrogels were shrunk back down using NaCl for post-imaging. eYFP was almost totally quenched, but this effect was likely mediated by the 1 M NaCl concentration of the buffer used to shrink samples for quantitative imaging. Nonetheless, the data demonstrate an experimental consequence of eYFP's chloride sensitivity that makes it unsuitable for many applications, in contrast to hfYFP, which unlike most YFPs, is chloride-insensitive (FIG. 1F). Thus, the thermal and chemical stability advantages of hfYFP and mGL resulted in stronger proExM performance.

### Example 6. Fixative Resistance

Likewise, the improvements to chemical and thermodynamic stability translated to conditions directly relevant to cells and tissue—namely resistance to fixatives used in histology and electron microscopy. Paraformaldehyde (PFA), glutaraldehyde (Glut), and glyoxal (ethanedial) are the most common histological preservatives in biology, and an FP capable of retaining substantial fluorescence in these fixatives would have considerable advantages in all downstream optical applications, particularly when factored together with expression-driven cellular brightness levels (FIG. 9C).

In all cases (except for the condition with only 4% PFA, in which eGFP was well preserved), the fluorescence retained by cells expressing hfYFP or mGL after fixative exposure was greater than that of the eGFP, mClover3, mNeonGreen, and eYFP cells (FIGS. 3B-3F). Consistent with the biochemical stability data, eYFP and mNeonGreen underperformed in most conditions. Differences between mhYFP and hfYFP in these assays were minimal but not negligible.

hfYFP retained nearly 80% of its fluorescence in the 2% PFA/2% Glut condition, a 360% improvement over eGFP and a 750% improvement over mNeonGreen (FIG. 3B). mGL was sensitive to glutaraldehyde (FIGS. 3B-3C), but it retained 23%, 180%, and 300% greater fluorescence after 4% PFA fixation than eGFP, mClover3, and mNeonGreen, respectively (FIG. 3D). eYFP was severely quenched by glyoxal. Although glyoxal is typically applied in a 20% ethanol solution at pH 4.0-5.0, the eYFP fluorescence could not be recovered after multiple washouts using PBS, pH 7.4 (FIG. 3Ee). The data highlight the considerable diversity of responses to aldehyde fixatives between FPs and demonstrate the degree to which experiments are impacted by fluorescence quenching at the earliest stage of sample processing.

### Example 7. Correlative Light and Electron Microscopy (CLEM)

For use in conditions compatible with electron microscopy, FPs must not only survive primary aldehyde fixation, but they must also thrive in the presence of secondary fixatives and electron-dense reagents. Primary among such reagents is osmium tetroxide (OsO₄), which is electron-dense and cross-links both proteins and unsaturated lipids. hfYFP and mhYFP tolerated doses of OsO₄ matched only by mEos4b, a green-to-red photoswitchable FP specifically engineered for OsO₄ resistance for CLEM. We tested the same FPs that we compared in the other stability assays and found that none could tolerate OsO₄ concentrations used for CLEM except mEos4b, hfYFP, and mhYFP (FIG. 4A). In a time-course experiment with FPs incubated for 1 hr in 1% OsO₄ (the concentration most often used for sample preservation), hfYFP retained greater fluorescence than mEos4b and mhYFP, while all other FPs—including sfGFP—were almost totally quenched before the first 10 min time point (FIG. 4B).

We subjected eGFP, mGL, and hfYFP to a modified EM fixation protocol (4% PFA and 0.2% Glut, followed by 1% OsO4 and embedding in agarose-optimal cutting temperature (OCT) compound—i.e., polyvinyl alcohol and polyethylene glycol) and quantified the retained fluorescence (FIG. 4C). HEK293 cells were transduced with adeno-associated virus (AAV) particles and live fluorescent cells were imaged by confocal microscopy at 10× (FIG. 4D) and 63× magnification (FIG. 4E) before and after processing, using the same settings. The processed hfYFP cells retained ~35% of the initial live cell fluorescence (FIG. 4F), a 14- and 25-fold fluorescence retention improvement compared to mGL and eGFP, respectively (FIG. 4G). hfYFP also retained fluorescence after acrylate-based resin embedding in a high-pressure freezing/freeze substitution protocol, with fluorescence levels well above background (FIG. 4h).

### Example 8. Crystal Structures

Hyperfolder YFP (hfYFP), monomeric hyperfolder YFP (mhYFP), and FOLD6, crystallized with the symmetry of space groups C222₁, C222₁, and P6₄, diffracting to 1.7 Å, 1.6 Å, and 1.2 Å, respectively. Each FP crystallized as a monomer with no asymmetric unit. Structures were solved by molecular replacement using homology models generated from Clover (PDB: 5WJ2; (Campbell et al., 2018)) (Table 4).

hfYFP features a chromophore hydrogen bond (H-bond) network indistinguishable from that of eYFP, Citrine, or Venus, besides trivial H-bond distance variation (±0.1-0.3 Å). In each of those structures, E222 O_{ε2} is H-bonded to N2 of the chromophore (CRO) imidazolinone ring, while E222 O_{ε1} is H-bonded to a structurally conserved water molecule, which we will rotor to wat₂. Wat₂ is H-bonded to the backbone amide of L68 and, as we will discuss later, it also connects E222 O_{ε1} to the Y203 phenolic side chain (FIG. 17A). By contrast, this Y203-wat₂-E222 O_{ε1} connection is broken in mhYFP, with wat₂ situated 3.7 Å away from E222 O_{ε1}, too far for an H-bond. The electron density of this 1.6 Å resolution structure supports a single E222 conformer whose side chain is rotated so that O_{ε1} H-bonds to [N2], while O_{ε2} H-bonds to S205, and S205 H-bonds to wat₁, H148 N_{δ1}, and N146 C═O. Although the chromophore phenolate remains stabilized by H-bonds from wat₁ and H148, this new completed proton wire through E222 (FIG. 17A) might further stabilize the structure while functionally decoupling the Y203 phenolate from the spectroscopically critical E222 side chain. We have not observed this conformation in eYFP (PDB ID: 1YFP (Wachter et al., 1998)), Venus (PDB ID: 1MYW (Rekas et al., 2002)), or Citrine (PDB ID: 1HUY (Griesbeck et al., 2001)) (FIG. 18A), perhaps due to the resolution limits of those structures (2.5 Å, 2.2 Å, and 2.2 Å, respectively). Interestingly, we also observe a single E222 conformer in our 1.2 Å FOLD6 structure (FIG. 17B).

We discuss our three crystal structures, similarities and differences to other common FPs (FIG. 17C), mechanisms of action for individual mutations including those that accelerate chromophore maturation (FIG. 17D), the functional and structural impact of the Q69M mutation (FIG. 18B), the destabilizing effects of the C48S/C70V substitutions (FIGS. 18C-18D), and the possibility surface electrostatic interactions (FIG. 19).

### Example 9. Structure-Guided Engineering of Fluorescent Proteins with a Large Stokes Shift

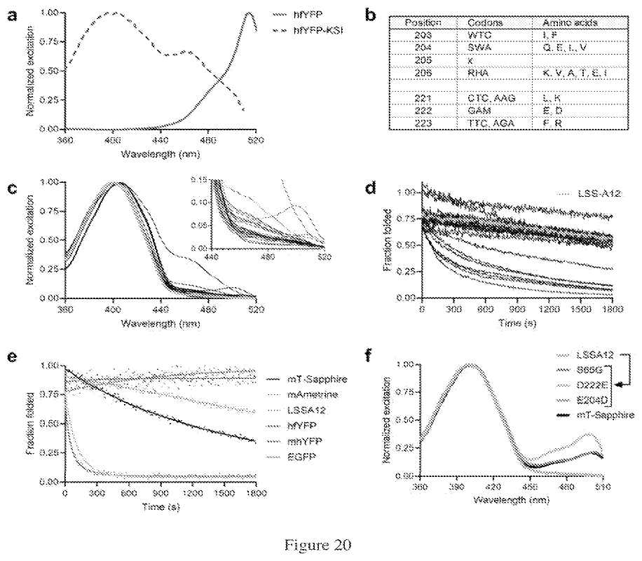

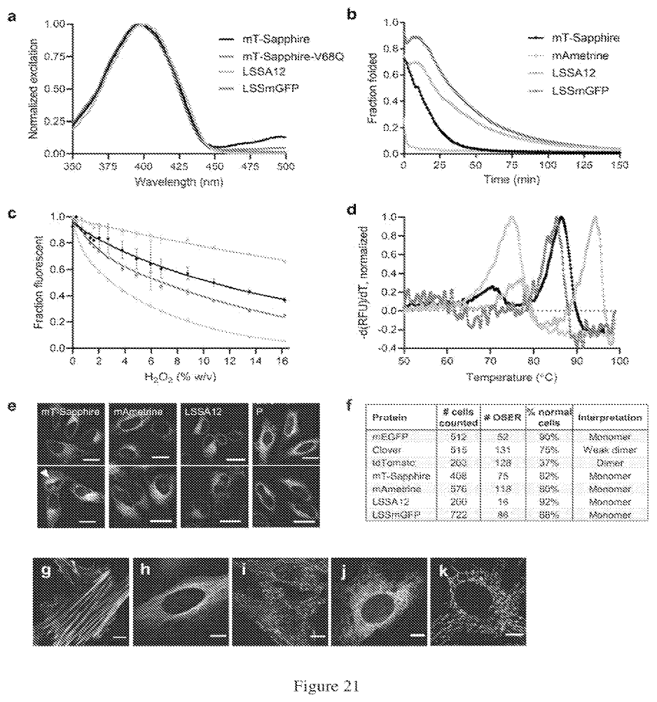

Having examined structure-activity relationships (SAR) in our hfYFP, mhYFP, and FOLD6 crystal structures (FIGS. 17 and 18), we sought to generate companion hyperfolder FPs for spectral multiplexing that could be excited exclusively by 405 nm illumination. We destabilized the deprotonated chromophore (“B-state”) (FIG. 17A) by introducing the T65S/Y203I mutations found in mT-Sapphire that are primarily responsible for its 405 nm excitation band (Zapata-Hommer and Griesbeck, 2003), essentially producing an hfYFP/mT-Sapphire chimera. Our hfYFP-G65S/Y203I/V206K mutant was 405 nm excitable, indicating a protonated chromophore population, but it still retained an excitation band at 460 nm (FIG. 20A).

We designed a structurally targeted library to eliminate the B-band by mutating positions 203, 204, 206, 221, 222, and 223. These residues on β-strands 10 and 11, especially those at positions 203 and 222, are crucial for the orientation of chromophore-associated side chains such as S205 that participate in excited-state proton transfer (ESPT) and directly influence the FP's spectral properties (FIGS. 5A-5B). We mutated the six residues simultaneously using a Gibson Assembly overlap approach using oligonucleotides carrying specifically selected degenerate codons to minimize library size to 384 combinations (FIG. 20B).

After screening E. coli colonies by eye for high 405 nm and low 470 nm excitation, spectroscopic analysis revealed mutants with B-band excitation that was dramatically reduced compared to hfYFP-G65S/Y203I (FIG. 20C). We extracted soluble protein from a subset of mutants and denatured the clarified lysate using the same GdnHCl kinetic unfolding strategy we employed to identify hfYFP. After a single round of screening, a mutant with an optimal combination of high GdnHCl stability (FIG. 20D) and no B-band excitation was sequenced, yielding hfYFP-G65S/Y203I/Q204E/E222D/R223F, which we named LSSA12. LSSA12 was more stable in GdnHCl than mT-Sapphire, mAmetrine, and eGFP (FIG. 20E). Site-directed mutagenesis confirmed the functional importance of the E222D and G65S mutations in LSSA12 (FIG. 20F).

Since hfYFP can tolerate avFP knockout substitutions (FIG. 16), we produced a diversified LSSA12 library by purifying the 12 best plasmids from the LSSA12 screen, diluted the DNA with original template to back-cross the library, and recombined the genes in a high-error rate staggered extension process (StEP) reaction spiked with 0.5 mM MnCl₂. The most stable clones with optimal excitation spectra and high GdnHCl stability were sequenced, yielding LSSmGFP, which is hfYFP-T43S G65S/L68Q/H77N/K140N/Y203I/V206K. Both LSSA12 and LSSmGFP show no B-band excitation, in contrast to mT-Sapphire (FIG. 21A). LSSmGFP persisted longer in GdnHCl than LSSA12, mT-Sapphire, and mAmetrine (FIG. 21B).

To confirm the importance of Gln68, we mutated mT-Sapphire to generate mT-Sapphire-V68Q (mT-Sapphire2, which we did not characterize further) and confirmed that V68Q alone was sufficient to decrease mT-Sapphire's residual 488 nm excitation dramatically (2.8-fold lower), almost to zero (FIG. 21A). Therefore, the V68Q substitution is a novel and apparently generalizable solution to enhance spectral tuning in 405 nm excitable avFPs.

LSSmGFP and LSSA12 are more acid-tolerant than mAmetrine (pKa=6.3) and mT-Sapphire (pKa=5.1), having pKa values of 4.6-4.7 (Table 5). They are also more resistant to H₂O₂ denaturation (FIG. 21C). Their melting temperatures (Tm) are high, at 84.8° C. and 93.9° C., respectively, comparable to sfGFP's and hfYFP's (FIG. 21D, Table 5). Cells transfected with LSSmGFP retained 72% of their live-cell fluorescence after fixation with 4% PFA, compared to 75% retained fluorescence for LSSmGFP, 62% for mT-Sapphire, and only 41% for mAmetrine (FIG. 21E). Like hfYFP, LSSmGFP and LSSA12 surpassed the stability of other FPs in glutaraldehyde (FIG. 21F) (FIG. 24). Under laser-scanning confocal illumination, LSSmGFP's photostability is comparable to mT-Sapphire's and twice as long as mAmetrine's (FIG. 21F), while its molecular brightness is roughly equal (Table 5).

LSSA12 and LSSmGFP, as well as mT-Sapphire and mAmetrine, behaved as monomers in cultured cells (FIG. 21H-21I). LSSmGFP localized properly to common intracellular targets (LSSA12 was not tested) (FIG. 21J-21N). LSSmGFP and LSSA12 enjoy similar advantages as hfYFP including the absence of cysteine residues, low pKa, tolerance of fixatives, high chemical and thermal stability, and a single excitation band.

Based on the clean excitation spectra of LSSmGFP and LSSA12, we hypothesized that these FPs would perform well in cells when co-expressed with mGreenLantern (FIG. 5C) and that mT-Sapphire and eGFP would show bleed-through between the 405 nm and 470 nm channels (FIG. 5D). We transfected eGFP & mT-Sapphire or mGL & LSSmGFP into HeLa cells as actin or H2B fusions, respectively. As expected, 470 nm widefield excitation inappropriately co-excited mT-Sapphire & eGFP, whereas LSSmGFP & mGL were properly separated into their respective channels with no bleed-through (FIG. 5E). Along with greater thermostability and chemical stability, LSSmGFP and LSSA12 offer improved excitation profiles relative to mT-Sapphire, without cross-excitation artifacts.

### Example 10. Fluorescence-Assisted Benchtop Purification of Inclusion Body Proteins Under Fully Denaturing Conditions

Most human proteins are insoluble when expressed in E. coli at 37° C. and are sequestered into inclusion bodies (IBs), which must be extracted and purified under chaotropic conditions typically involving buffers of 6 M GdnHCl. We aimed to exploit the high GdnHCl stability of hfYFP (FIGS. 2A and 2C) and LSSmGFP (FIG. 21B) by using them as fusion tags to enable fluorescence-aided protein purification under denaturing conditions using immobilized metal affinity chromatography (IMAC).

We generated constructs of hexahistidine-tagged (His₆) eGFP, hfYFP, or LSSmGFP, with the FP separated from mScarlet-I, Bacillus circulans xylanase (Bcx), or streptavidin (SAV) (collectively, Proteins of Interest, or POIs) by a linker containing a TEV protease cleavage site (FIG. 5G). We developed a purification protocol using the hfYFP-mScarlet fusion construct (FIG. 22) and then purified SAV fusions of eGFP, hfYFP, and LSSmGFP under denaturing conditions using Ni-NTA chromatography with all solutions containing 6 M GdnHCl. We successfully visualized every stage of bacterial lysis, inclusion body isolation, solubilization, purification, refolding during dialysis, proteolytic cleavage and isolation of the fusion protein using inexpensive 405 nm and 470 nm LED strips and orange filter glasses. Elution was monitored by eye using LED illumination at the benchtop. The purified SAV was functional, exhibiting 33% binding activity relative to purified SAV obtained from a commercial source (FIG. 5H). As expected from the kinetic unfolding assays, eGFP immediately denatured in GdnHCl during the inclusion body solubilization step (FIG. 5F, arrow) while hfYFP and LSSmGFP remained fluorescent.

Interestingly, hfYFP and LSSmGFP appear to function as solubility tags that enhance expression of the C-terminal fusion protein. When fusions were expressed in E. coli at 37° C. under strong induction conditions to maximize inclusion body formation, we obtained 30% and 60% less mScarlet-I in the insoluble fraction (FIG. 6) and 290% and 220% more mScarlet-I in the soluble fraction from the hfYFP- and LSSmGFP-mScarlet fusions, respectively, than from the eGFP-mScarlet construct. Compared to the eGFP-Bcx fusion, 820% and 800% more fusion protein was obtained from the soluble fraction of hfYFP and LSSmGFP fusions, while a marginal 10% and 20% more SAV was collected. In the clarified LB medium (without cells), 930% and 470% more mScarlet-I fusion protein was detectable for the hfYFP and LSSmGFP constructs, respectively, compared to eGFP. Almost no eGFP-Bcx fusion construct was detectable in the media, while high amounts were visible for the hfYFP and LSSmGFP fusions (FIG. 6).

Altogether, fusing an insoluble protein (or a soluble one, in the case of mScarlet-I) to hfYFP or LSSmGFP generated much more soluble and less insoluble fusion protein than the equivalent eGFP fusions for three diverse natural proteins that have no appreciable similarity in sequence or structure. None of the C-terminal fusion proteins contain cysteines, so the improvement cannot be attributed to eliminating disulfide-mediated aggregation in the Proteins of Interest. hfYFP and LSSmGFP have potential to serve as solubility tags or at least as markers for fluorescence-assisted protein purification in addition to the numerous applications that we have described for proExM, CLEM, and protein engineering.

### Example 11. Equilibrium Unfolding

We determined the GdnHCl concentration corresponding with the half-maximal fluorescence value (C_{1/2}) for each FP and found that sfGFP showed only a minor rightward curve shift relative to eGFP, with C_{1/2}=4.0 and 4.3 M, respectively. Likewise, mClover3 (C_{1/2}=3.8 M) was only slightly less stable than eGFP in GdnHCl, whereas mNeonGreen and eYFP showed the lowest values of C_{1/2}=2.0 and 1.2 M, respectively. mGL displayed the same curve shape as the control FPs, except that its stability was far greater, at C_{1/2}=5.5 M, a dramatic improvement in chemical stability disproportionate to its Tₘ value (FIG. 2C). In the more chaotropic GdnSCN solutions, mGL and hfYFP outperformed other FPs, showing C_{1/2}=3.2 and 2.3 M, respectively, while C_{1/2} values for eGFP, sfGFP, mClover3, and eYFP were no greater than 0.2 M (FIG. 2D) (Table 3).

### Example 12. Discovery of the S147R Mutation

While mutating mF4Y (Table 1) to produce an S147P variant, we fortuitously identified a de novo S147R mutation that dramatically accelerates chromophore maturation. mF4Y-S147R has the fastest maturation rate of any of our mutants except mF1Y (contains N149Y) and mF2BK^{DMD} (contains N149K) with half-times of 6 min and 7 min, respectively (Table 2). Structurally, the S147R mutation replaces the small polar serine side chain with a volumetrically larger but also more flexible charged residue in a section of the barrel that is well-described as the most heterogeneous. It is conceivable that the S147R side chain could form cation-n stacking interactions with Y149 in the mF4Y protein or other electrostatic interactions with neighboring residues to offer new conformational options during protein folding. In any case, our data demonstrate that the composition of β-strand 7 is an integral feature of the chromophore maturation process.

### Example 13. Fluorescent Proteins with a Reduced Genetic Code

Since the hyperfolder proteins readily tolerate cysteine substitutions that severely diminish eGFP and sfGFP fluorescence, we asked whether the indispensable tryptophan residue that is conserved in all avFPs could now be substituted as well (FIG. 16A). Trp57 stabilizes the avFP hydrophobic core including the central helix “PVPWP” motif. All W57 substitutions produce insoluble, nonfluorescent protein except for the eGFP-W57F mutant that retains only 5% of wild-type eGFP's brightness.

We generated W57F point mutants of eGFP, sfGFP, mF4P (FIG. 8), and hfYFP, and confirmed that the absorbance spectra were unchanged (FIG. 16B). Like eGFP-C48S/C70V (FIG. 7E), eGFP-W57F was almost entirely nonfluorescent in E. coli. On the other hand, 30% of sfGFP fluorescence and 84% of hfYFP fluorescence was preserved in their W57F mutants (FIG. 16F). Here, too, brightness in bacteria corresponded with greater soluble protein production: hfYFP-W57F was 2.5-fold brighter than wild-type sfGFP and 7-fold brighter than sfGFP-W57F (FIG. 16G). In all cases, the W57F mutation did not change the excitation and emission maxima, QY, or EC (FIG. 16H).

Even with an 18 amino acid genetic code resulting from three deleterious substitutions (C48S/W57F/C70V), hfYFP-W57F was much more stable than even wild-type sfGFP. hfYFP-W57F persisted in GdnSCN 3.6 M for minutes before denaturing (FIG. 16D); it remained fluorescent more than 4 times longer than wild-type sfGFP during isothermal melting at 80° C. (FIG. 16C); and hfYFP-W57F's melting temperature was still greater than wild-type sfGFP's (Tₘ=89.1 vs. 86.4° C., respectively) (FIG. 16E). The data collectively demonstrate that hfYFP is equipped to tolerate major structural perturbations such as those induced by random mutagenesis, due to its assortment of compensatory folding mutations and thermodynamic stability benefits compared to eGFP and sfGFP.

### Example 14. NaOH Resistance and Development of mhYFP

While experimentally determining FP extinction coefficients (ECs) using the alkali denaturation method, we noted that several FPs were not showing the single distinct ~447 nm peak absorbance peak of the alkali denatured chromophore. In contrast to Clover (FIG. 14A), sfGFP (FIG. 14B), and all other avFPs we had tested, FOLD6 required 10 min to display the denatured chromophore's 447 nm absorbance peak without overlap from its 505 nm native peak in this 1 M NaOH solution (pH≥13) (FIG. 14C). We performed time-course experiments with 8 different FPs and found that Clover, eYFP, and even mGreenLantern and hfYFP, denatured right away (FIGS. 14D-14G). By contrast, mF4P (FOLD4-S147P), like FOLD6 (FOLD4-S147P/V68L/L221K) persisted for several minutes in NaOH (FIGS. 14H-14I), suggesting a role of the S147P mutation (Table 2) that we had incorporated into our mutants due to its reported thermostability benefits. mF4Y-SR (FOLD4-S147R/N149Y) also showed delayed denaturation, to a lesser degree (FIG. 14J), suggesting that mutations to β-strand 7, the most conformationally heterogeneous strand of avFPs, were more broadly responsible for the effect.

We attempted to bestow this NaOH resistance into hfYFP using the S147P mutation (FIG. 8) and introduced the V206K mutation to improve monomericity. V206K improved the OSER score (FIGS. 12B-12C) and did not disrupt GdnHCl stability when tested in multiple mutants (FIG. 15). The S147P mutation conferred mnF4P-like NaOH persistence to mhYFP. mhYFP persisted longer than mF4Y-SR in NaOH but not for as long as FOLD6 did (FIG. 14K). Thus, structure-guided engineering of β-strand 7 yielded monomeric hyerfolder YFP (mhYFP) with enhanced resistance to NaOH and/or extreme alkaline conditions (pH≥13), further demonstrating that peculiar performance features can be engineered into FPs without perturbing spectral properties (Table 2 and Table 3). The data demonstrate that hfYFP exhibits uncanny stability in diverse chaotropic conditions that rapidly degrade most biological structures in seconds to minutes.

### Example 15. Structure-Activity Relationships in hfYFP, mhYFP, and FOLD6

In our FOLD6 structure (1.21 Å resolution), the H203 side chain is decoupled from a single-conformer E222 by an intervening water molecule, which we refer to as wat₃, bridging the gap between S205 and E222. As we have shown previously in the Clover structure, Clover's E222 O_{ε1} associates with the H203 N_{δ1} atom through a 2.7 Å H-bond, thereby eliminating the S205-E222 H-bond that sustains the minor protonated chromophore population observed in eGFP high-resolution structures (PDB: 4EUL), which manifests as a 405 nm absorbance band. In FOLD6, affinity of E222 for neighboring wat₃ (3.1 Å distance) has withdrawn E222 from H203 and brought it closer to β-strand 11, eliminating this Clover-like H-bond (FIG. 17B).

Consequently, H203 is shifted even farther away from E222 relative to Clover, placing the H203 N_{δ1} atom directly above and tilted 21° into the Y66 centroid relative to the ring edge nearest F165, a parallel-displaced π-stacking interaction almost identical to that found in the disulfide-oxidized structure of roClover0.1 (Clover-S147C/Q204C is a redox biosensor template we reported previously; ibid). Perhaps for this reason, the FOLD6 chromophore is far more planar than hfYFP's where, by contrast, the centrosymmetric π-stacking of Y66-Y203 leaves the Y66 ring noticeably rotated about the methylene bridge relative to the imidazolinone moiety to achieve greater coplanarity with the Y203 ring. Deviations from planarity between the chromophore imidazolinone and phenolate moieties are well known to decrease quantum yield.

The highly off-center H203 orientation relative to Y66 considerably widens a separation between β-strands 7 and 8 in FOLD6 through steric effects on H203. Compared to Clover, the H203 side chain has shifted 0.5 Å away from E222 and toward V150, pushing the V150 side chain and local β-strand 7 backbone 1.0-1.3 Å outward and away from H203. This new V 150 position forces the F165 side chain down 0.6 Å toward H181 and the F165 C_{α} carbon 1.0 Å outward, while the neighboring N164 carbonyl rotates more than 25° away from the Y151 amide, severing the β-strand H-bond between the two (linear distances of 3.1 Å in sfGFP and 5.1 Å in FOLD6). hfYFP shows similar conformational changes that fall between those of sfGFP and FOLD6. We speculate that these changes might facilitate chromophore maturation by increasing the porosity of barrel sections adjacent to catalytically important residues during protein folding and chromophore cyclization steps.

In all three of our structures, the C48 and C70 side chains are replaced with serine and valine, respectively, and the same conformations are observed in all. The C48S substitution eliminates sulfur-aromatic and vdW interactions with W57 and F27 and weakens or extinguishes numerous long-range weakly polar interactions with the loop main-chain atoms, instead producing a 2.3 Å H-bond between the S48 side-chain and G51 carbonyl (FIG. 18C). On the opposite side of the chromophore and along the central helix, C70 coordinates vdW forces and sulfur-aromatic interactions with F8, F71, and Y92. These interactions are eliminated in the C70V mutants and are largely replaced by vdW forces, with the shortest distances observed between V70 and F8 side-chains (FIG. 18D). Overall, the C48S and C70V mutations likely destabilize these micro-regions, although computational comparisons would help verify this assertion.

Hyperfolder YFP combines two distinct features of Venus (PDB: 1MYW) and Citrine (PDB: 1HUY): the Q69M mutation, and the F46L/F64L mutations, respectively. In hfYFP and Venus, the F46L/F64L mutations eliminate the antiparallel π-π stacking interaction present in eGFP and the closely related structures, eYFP and Venus. Consequently, the smaller alkyl side-chains of L44, L42, and L220, among other residues that line the protein's hydrophobic core (approximately bordered by N121), shift to occupy the space (FIG. 17C).

The Q69M mutation from Citrine substitutes a polar side chain with the hydrophobic yet weakly polar and larger methionine, which packs more efficiently into the chromophore cavity and is known to improve chromophore maturation. The conformations of M69 in hfYFP and Citrine structures overlap due to extensive side-chain packing (FIG. 17C): M69 is situated directly between three aromatic residues, not including the chromophore's π-conjugated system itself, and M69 shows notable orientational preference for the δ⁺ ring edge of F84, whose centroid is located approximately 5 Å away with the δ⁻ M69 sulfur atom and a π-cloud presumably oriented 90° relative to the ring (FIG. 18B). This configuration indicates a sulfur-aromatic interaction that should contribute stabilizing energy greater than the sum of the vdW forces. Additionally, the Y203 centroid and the chromophore imidazolinone ring are approximately 6 Å from M69 S_{δ}, suggesting that the sulfur atom's lone pair electrons may stabilize those features as well. Altogether, M69 serves a variety of functions that the wild-type glutamine side-chain cannot, including greater hydrophobic packing and long-range stabilizing interactions with the aromatic residues that may improve the FP's overall stability.

### Example 16. Salt Bridges on the Hyperfolder Protein Surfaces

It has been shown that the S30R superfolder mutation enables salt bridges between several residues on the protein surface that contribute to its stability, and we observe extensive electrostatic interactions across β strands 1, 2, 5, and 6, in FOLD6, hfYFP, and mhYFP (FIG. 19). hfYFP shows a single R30 conformation through which four different side-chains are stabilized (FIG. 19D), whereas mhYFP shows two R30 conformations, shifting the equilibrium more toward stabilization of E32 than D19 (FIG. 19E). On the other hand, R30 in FOLD6 also shows two conformations, but the rotamers allow R30 to stabilize E32 and D19 via salt bridges and to an H-bond to S28 (FIG. 19C). These electrostatic surface interactions may contribute to the stability of the hyperfolder FPs.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

What is claimed is:

1. A fluorescent protein comprising at least 90%, 95%, 96%, 97%, 98%, 99% or 100% of the amino acid sequence selected from SEQ ID NOs: 1, 3, 5, 7-10, and 12.

2. The fluorescent protein of claim 1, wherein the fluorescent protein comprises the amino acid sequence of SEQ ID NO: 1.

3. The fluorescent protein of claim 1, wherein the fluorescent protein comprises the amino acid sequence of SEQ ID NO: 3.

4. The fluorescent protein of claim 1, wherein the fluorescent protein comprises the amino acid sequence of SEQ ID NO: 5.

5. The fluorescent protein of claim 1, wherein the fluorescent protein comprises the amino acid sequence of SEQ ID NO: 7.

6. The fluorescent protein of claim 1, wherein the fluorescent protein comprises the amino acid sequence of SEQ ID NO: 8.

7. The fluorescent protein of claim 1, wherein the fluorescent protein comprises the amino acid sequence of SEQ ID NO: 9.

8. The fluorescent protein of claim 1, wherein the fluorescent protein comprises the amino acid sequence of SEQ ID NO: 10.

9. The fluorescent protein of claim 1, wherein the fluorescent protein comprises the amino acid sequence of SEQ ID NO: 12.

10. The fluorescent protein of any one of claims 1-9, wherein the fluorescent protein is chloride insensitive.

11. A fusion protein comprising a fluorescent protein according to any one of claims 1-10.

12. The fusion protein of claim 11, wherein the fluorescent protein is linked to a polypeptide.

13. A nucleic acid molecule encoding the fluorescent protein according to any one of claims 1-10.

14. A nucleic acid molecule encoding the fusion protein according to any one of claims 11-12.

15. A nucleic acid molecule comprising the nucleotide sequence selected from SEQ ID NOs: 2, 4, and 6.

16. The nucleic acid molecule of any one of claims 13-15, wherein the nucleic acid molecule is a cDNA.

17. A vector comprising the nucleic acid molecule according to any one of claims 13-16.

18. A host cell comprising the nucleic acid molecule of any one of claims 13-16.

19. A host cell comprising the vector of claim 17.

20. A host cell comprising the fluorescent protein of any one of claims 1-10.

21. A host cell comprising the fusion protein of any one of claims 11-12.

22. The host cell of any one of claims 18-21, wherein the host cell is a mammalian host cell.

23. A method of detecting the expression of a protein of interest in a cell, the method comprising:
i) introducing into a cell a nucleic acid molecule comprising a nucleotide sequence encoding a protein of interest fused to a nucleotide sequence encoding the fluorescent protein according to any one of claims 1-10;
ii) culturing the cell under conditions suitable for the expression of said protein of interest; and
iii) detecting the expression of said protein of interest by measuring the fluorescence of said cell.

24. The method of claim 23, wherein said nucleic acid molecule being operatively linked and under the control of a suitable expression control sequence.

25. The method of claim 23 or 24, wherein the fluorescence is measured by optical means.

26. The method of any one of claims 23-25, wherein the optical means is fluorescent microscopy, flow cytometry, spectroscopy, laser-scanning confocal microscope, confocal microscope, correlative light and electron microscopy, or protein-retention expansion microscopy.

27. A method of purifying a protein of interest, the method comprising:
i) introducing into a cell a nucleic acid molecule comprising a nucleotide sequence encoding a protein of interest fused to a nucleotide sequence encoding the fluorescent protein according to any one of claims 1-10;
ii) culturing the cell under conditions suitable for the expression of said protein of interest;
iii) lysing the cell; and
iii) purifying the protein of interest by purifying the fluorescent protein.

28. The method of claim 27, wherein said nucleic acid molecule being operatively linked and under the control of a suitable expression control sequence.

29. The method of claim 27 or 28, wherein the fluorescent protein is purified by organic extraction or Fluorescence ImmunoPrecipitation (FLIP).


SEQUENCE LISTING (24 sequences)

SEQ ID NO: 1 - protein, 239 aa, synthetic construct
MVSKGEELFTGVVPILVELDGDVNGHKFSVRGEGEGDATNGKLTLKLISTTGKLPVPWPTLVTTLGYGLMVFARYPDHMKQHDFFKSAMPEGYVQERTISFEDDGYYKTRAEVKFEGDTLVNRIVLKGIDFKEDGNILGHKLEYNFNSHNVYITADKQKNGIKANFKIRHNVEDGGVQLADHYQQNTPIGDGPVLLPDNHYLSYQSVLSKDPNEKRDHMVLKERVTAAGITHDMNELYK

SEQ ID NO: 2 - DNA, 717 bp, synthetic construct
  source 1..717: mol_type = other DNA
atggtgagcaagggcgaggagctgttcaccggggtggtgcccatcctggtcgagctggacggcgacgtaaacggccacaagttcagcgtccgcggcgagggcgagggcgatgccaccaacggcaagctgaccctgaagctcatctccaccaccggcaagctgcccgtgccctggcccaccctcgtgaccaccttaggctacggcctgatggtgttcgcccgctaccccgaccacatgaagcagcacgacttcttcaagtccgccatgcccgaaggctacgtccaggagcgcaccatctctttcgaggacgacggttactacaagacccgcgccgaggtgaagttcgagggcgacaccctggtgaaccgcatcgtgctgaagggcatcgacttcaaggaggacggcaacatcctggggcacaagctggagtacaacttcaacagccacaacgtctatatcacggccgacaagcagaagaacggcatcaaggctaacttcaagatccgccacaacgttgaggacggcggcgtgcagctcgccgaccactaccagcagaacacccccatcggcgacggccccgtgctgctgcccgacaaccactacctgagctaccagtccgtcctgagcaaagaccccaacgagaagcgcgatcacatggtcctgaaggagagggtgaccgccgccgggattacacatgacatgaacgagctgtacaag

SEQ ID NO: 3 - protein, 239 aa, synthetic construct
MVSKGEELFTGVVPILVELDGDVNGHKFSVRGEGEGDATNGKLTLKLISTTGKLPVPWPTLVTTLGYGLMVFARYPDHMKQHDFFKSAMPEGYVQERTISFEDDGYYKTRAEVKFEGDTLVNRIVLKGIDFKEDGNILGHKLEYNFNPHNVYITADKQKNGIKANFKIRHNVEDGGVQLADHYQQNTPIGDGPVLMPDNHYLSYQSKLSKDPNEKRDHMVLKERVTAAGITHDMNELYK

SEQ ID NO: 4 - DNA, 717 bp, synthetic construct
  source 1..717: mol_type = other DNA
atggtgagcaagggcgaggagctgttcaccggggtggtgcccatcctggtcgagctggacggcgacgtaaacggccacaagttcagcgtccgcggcgagggcgagggcgatgccaccaacggcaagctgaccctgaagctcatctccaccaccggcaagctgcccgtgccctggcccaccctcgtgaccaccttaggctacggcctgatggtgttcgcccgctaccccgaccacatgaagcagcacgacttcttcaagtccgccatgcccgaaggctacgtccaggagcgcaccatctctttcgaggacgacggttactacaagacccgcgccgaggtgaagttcgagggcgacaccctggtgaaccgcatcgtgctgaagggcatcgacttcaaggaggacggcaacatcctggggcacaagctggagtacaacttcaacccccacaacgtctatatcacggccgacaagcagaagaacggcatcaaggctaacttcaagatccgccacaacgttgaggacggcggcgtgcagctcgccgaccactaccagcagaacacccccatcggcgacggccccgtgctgatgcccgacaaccactacctgagctaccagtccaagctgagcaaagaccccaacgagaagcgcgatcacatggtcctgaaggagagggtgaccgccgccgggattacacatgacatgaacgagctgtacaag

SEQ ID NO: 5 - protein, 239 aa, synthetic construct
MVSKGEELFTGVVPILVELDGDVNGHKFSVRGEGEGDATNGKLSLKLISTTGKLPVPWPTLVTTLSYGQMVFARYPDNMKQHDFFKSAMPEGYVQERTISFEDDGYYKTRAEVKFEGDTLVNRIVLKGIDFKEDGNILGHNLEYNFNSHNVYITADKQKNGIKANFKIRHNVEDGGVQLADHYQQNTPIGDGPVLLPDNHYLSIQSKLSKDPNEKRDHMVLKERVTAAGITHDMNELYK

SEQ ID NO: 6 - DNA, 717 bp, synthetic construct
  source 1..717: mol_type = other DNA
atggtgagcaagggcgaggagctgttcaccggggtggtgcccatcctggtcgagctggacggcgacgtaaacggccacaagttcagcgtccgcggcgagggcgagggcgatgccaccaacggcaaactgtccctgaagctcatctccaccaccggcaagctgcccgtgccctggcccaccctcgtgaccaccttaagctacggccagatggtgttcgcccgctaccctgacaacatgaagcagcacgacttcttcaagtccgccatgcccgaaggctacgtccaggagcgcaccatctctttcgaggacgacggttactacaagacccgcgccgaggtgaagttcgagggcgacaccctggtgaaccgcatcgtgctgaagggcatcgacttcaaggaggacggcaacatcctggggcacaacctggagtacaacttcaacagccacaacgtctatatcacggccgacaagcagaagaacggcatcaaggctaacttcaagatccgccacaacgttgaggacggcggcgtgcagctcgccgaccactaccagcagaacacccccatcggcgacggccccgtgctgctgcccgacaaccactacctgagcatccagtccaagctgagcaaagaccccaacgagaagcgcgatcacatggtcctgaaggagagggtgaccgccgccgggattacacatgacatgaacgagctgtacaag

SEQ ID NO: 7 - protein, 239 aa, synthetic construct; identical to SEQ ID NO: 3

SEQ ID NO: 8 - protein, 239 aa, synthetic construct
MVSKGEELFTGVVPILVELDGDVNGHKFSVRGEGEGDATNGKLTLKLISTTGKLPVPWPTLVTTLSYGLMVFARYPDHMKQHDFFKSAMPEGYVQERTISFEDDGYYKTRAEVKFEGDTLVNRIVLKGIDFKEDGNILGHKLEYNFNSHNVYITADKQKNGIKANFKIRHNVEDGGVQLADHYQQNTPIGDGPVLLPDNHYLSIQSKLSKDPNEKRDHMVLKERVTAAGITHDMNELYK

SEQ ID NO: 9 - protein, 239 aa, synthetic construct
MVSKGEELFTGVVPILVELDGDVNGHKFSVRGEGEGDATNGKLTLKLISTTGKLPVPWPTLVTTLSYGLMVFARYPDHMKQHDFFKSAMPEGYVQERTISFEDDGYYKTRAEVKFEGDTLVNRIVLKGIDFKEDGNILGHKLEYNFNSHNVYITADKQKNGIKANFKIRHNVEDGGVQLADHYQQNTPIGDGPVLLPDNHYLSIQSVLSKDPNEKRDHMVLKERVTAAGITHDMNELYK

SEQ ID NO: 10 - protein, 239 aa, synthetic construct
MVSKGEELFTGVVPILVELDGDVNGHKFSVRGEGEGDATNGKLTLKLISTTGKLPVPWPTLVTTLSYGLMVFARYPDHMKQHDFFKSAMPEGYVQERTISFEDDGYYKTRAEVKFEGDTLVNRIVLKGIDFKEDGNILGHKLEYNFNSHNVYITADKQKNGIKANFKIRHNVEDGGVQLADHYQQNTPIGDGPVLLPDNHYLSIESVLSKDPNEKRDHMVLKDFVTAAGITHDMNELYK

SEQ ID NO: 11 - protein, 239 aa, synthetic construct
MVSKGEELFTGVVPILVELDGDVNGHKFSVSGEGEGDATYGKLTLKFICTTGKLPVPWPTLVTTFSYGVMVFARYPDHMKQHDFFKSAMPEGYVQERTIFFKDDGNYKTRAEVKFEGDTLVNRIELKGIDFKEDGNILGHKLEYNFNSHNVYIMADKQKNGIKANFKIRHNIEDGGVQLADHYQQNTPIGDGPVLLPDNHYLSIQSKLSKDPNEKRDHMVLLEFVTAAGITLGMDELYK

SEQ ID NO: 12 - protein, 239 aa, synthetic construct
MVSKGEELFTGVVPILVELDGDVNGHKFSVSGEGEGDATYGKLTLKFICTTGKLPVPWPTLVTTFSYGQMVFARYPDHMKQHDFFKSAMPEGYVQERTIFFKDDGNYKTRAEVKFEGDTLVNRIELKGIDFKEDGNILGHKLEYNFNSHNVYIMADKQKNGIKANFKIRHNIEDGGVQLADHYQQNTPIGDGPVLLPDNHYLSIQSKLSKDPNEKRDHMVLLEFVTAAGITLGMDELYK

SEQ ID NO: 13 - protein, 6 aa, synthetic construct
HHHHHH

SEQ ID NO: 14 - protein, 7 aa, synthetic construct
GMDELYK

SEQ ID NO: 15 - protein, 7 aa, synthetic construct
DMDELYK

SEQ ID NO: 16 - protein, 7 aa, synthetic construct
DMNELYK

SEQ ID NO: 17 - protein, 7 aa, synthetic construct
MVSKGEE

SEQ ID NO: 18 - DNA, 21 bp, synthetic construct
  source 1..21: mol_type = other DNA
atggtgagcaagggcgaggag

SEQ ID NO: 19 - DNA, 21 bp, synthetic construct
  source 1..21: mol_type = other DNA
ggcatggacgagctgtacaag

SEQ ID NO: 20 - DNA, 21 bp, synthetic construct
  source 1..21: mol_type = other DNA
gacatggacgagctgtacaag

SEQ ID NO: 21 - DNA, 21 bp, synthetic construct
  source 1..21: mol_type = other DNA
gacatgaacgagctgtacaag

SEQ ID NO: 22 - protein, 20 aa, synthetic construct
GSAGSAAGSGEFENLYFQGH

SEQ ID NO: 23 - protein, 5 aa, synthetic construct
PVPWP

SEQ ID NO: 24 - protein, 239 aa, synthetic construct
MVSKGEELFTGVVPILVELDGDVNGHKFSVRGEGEGDATNGKLTLKFICTTGKLPVPWPTLVTTLGYGVACFARYPDHMKQHDFFKSAMPEGYVQERTISFKDDGTYKTRAEVKFEGDTLVNRIVLKGIDFKEDGNILGHKLEYNFNSHKVYITADKQKNGIKANFKTRHNVEDGGVQLADHYQQNTPIGDGPVLLPDNHYLSHQSKLSKDPNEKRDHMVLKERVTAAGITHDMDELYK